(19) United States
(12) Patent Application Publication
(10) Pub. No.: US 2026/0015414 A1
(43) Pub. Date: Jan. 15, 2026

(54) Methods of Treating Hidradenitis Suppurativa with Anti-IL-1beta Antibodies

(71) Applicant: Avalo Therapeutics, Inc., Rockville, MD (US)

(72) Inventors: Garry A. Neil, Havertown, PA (US); Colleen D. Matkowski, Chalfont, PA (US); Lisa Hegg, Devon, PA (US)

(73) Assignee: Avalo Therapeutics, Inc., Rockville, MD (US)

(21) Appl. No.: 19/089,129

(22) Filed: Mar. 25, 2025

Related U.S. Application Data
(60) Provisional application No. 63/670,250, filed on Jul. 12, 2024.
(60) Provisional application No. 63/695,639, filed on Sep. 17, 2024.
(60) Provisional application No. 63/762,771, filed on Feb. 25, 2025.

(51) Int. Cl. C07K 16/24

(52) U.S. Cl. CPC C07K 16/245

(57) ABSTRACT

The present disclosure relates to methods of treating hidradenitis suppurativa with anti-IL-1β antibodies.

### CROSS-REFERENCE TO RELATED APPLICATIONS

This patent application claims the benefit of priority to U.S. Provisional Patent Application No. 63/670,250, filed Jul. 12, 2024, U.S. Provisional Application No. 63/695,639, filed Sep. 17, 2024, and U.S. Provisional Application No. 63/762,771, filed Feb. 25, 2025, the contents of each of which are incorporated herein by reference in their entirety for all purposes.

### SEQUENCE LISTING

The instant application contains a Sequence Listing which has been submitted electronically in XML format and is hereby incorporated by reference in its entirety. Said XML copy, created on Mar. 3, 2025, is named “2025-03-03_01118-0070-00PCT_ST26.xml” and is 76,533 bytes in size.

### FIELD OF THE INVENTION

The invention relates to methods of treating or preventing hidradenitis suppurativa (HS), also known as acne inversa, with anti-interleukin-1β (IL-1β) antibodies.

### BACKGROUND OF THE INVENTION

Hidradenitis suppurativa is a chronic, recurrent inflammatory skin disease caused by obstruction of the ducts of the apocrine glands, after which the glands dilate and rupture. Secondary infection occurs, with the formation of abscesses and fistulas with branched trajectories. HS typically manifests as painful, deep-seated, inflamed lesions, including abscesses and nodules, most commonly in the axilla (armpit) and groin.

Lesions caused by HS increase in severity over time and may initially appear as painful subcutaneous nodules. As the condition progresses, the lesions comprise recurrent abscesses and sinus tracts and cause scarring. The lesions may converge over time leading to increased inflammation and scarring. In most patients, recurrent flares are accompanied by increased pain and suppuration. Chronic inflammation leads to fibrosis and the appearance of scar tissue. HS lesions are pruritic (itchy) and can be malodorous with a purulent discharge. If untreated, the flares generally require 7 to 10 days to subside. (See generally Saunte D & Jemec G, JAMA 2017; 318(20):2019-2032; Jemec G, N. Engl. J. Med. 2012; 366(2):158-164; Fabian O, et al., Ann. Ital. Chir. 2023; 12 (ISSN 2239-253X)).

Hidradenitis suppurativa has an estimated prevalence of 0.05% to 4.10% depending on the methodology, with lower estimates determined in registry studies and higher estimates based on prospective or self-reported studies. (Saunte D & Jemec G, JAMA 2017; 318(20):2019-2032). HS usually develops after puberty, but, can sometimes develop in infants.

Despite its prevalence, many patients receive inadequate treatment for hidradenitis suppurativa. Topical disinfectants are frequently used but there is little evidence to show that they are effective. Antibiotics have also been used to manage HS, and their efficacy has been attributed in part to their potential ability to modulate the immune system. (Saunte D & Jemec G, JAMA 2017; 318(20):2019-2032). Surgical interventions (incision and drainage, de-roofing) and intralesional steroids are utilized for recalcitrant wounds and local flares. Female patients with hormonal/cyclic exacerbations can be treated with anti-androgens such as spironolactone. (Alikhan A, Sayed C, Alavi A, et al., J Am Acad Dermatol. 2019; 81(1):76-90, Alikhan A, Sayed C, Alavi A, et al., J Am Acad Dermatol. 2019; 81(1):91-101.). Retinoids and steroids (topical, oral, and injections) are also currently used to manage HS.

Adalimumab, a monoclonal antibody that blocks TNF-α, was approved by the US Food and Drug Administration (FDA) in 2015 for the treatment of hidradenitis suppurativa. However, the efficacy of adalimumab was reported to be highly variable, and there remains a significant unmet need for an effective treatment for HS. (Zouboulis C, et al., Exp. Dermatology, 2021; 30 (Suppl. 1): 8-17; Martora F, et al., Clinical, Cosmetic and Investigational Dermatol., 2023; 16, 134-148). Indeed, it has been reported that many patients will experience primary or secondary failure of adalimumab. (Kanni, et al., J. Inv. Dermatology, 2018; 138, 795-801). Approximately 50% of adalimumab-treated patients do not reach Hidradenitis Suppurativa Clinical Response (HiSCR) 50. (Kimball A B, Okun M M, Williams D A, et al., N Engl J Med. 2016; 375(5):422-34; Krueger J G, Frew J, Jemec G B E, et al., Br J Dermatol. 2024; 190(2):149-162). Among patients who respond to adalimumab, loss of response due to the development of anti-drug antibodies (ADA) may be as high as 20% per patient year, as has been described in the gastrointestinal literature. (Billioud V, Sandborn W J, Peyrin-Biroulet., Am J Gastroenterol. 2011; 106(4):674-84; Abdalla T, Mansour M, Bouazzi D, et al. Am J Clin Dermatol. 2021; 22(2):275-83).

Secukinumab, a monoclonal antibody that blocks IL-17, was approved by the US FDA in 2023 to treat hidradenitis suppurativa. The efficacy of secukinumab for treatment of HS has been described as modest at least when measured in terms of HiSCR. (See Maul, et al., The Lancet 2024; 403(10427): 616-617). Data from clinical studies SUNSHINE and SUNRISE suggest that more than half of patients did not benefit from IL-17 directed inhibition with secukinumab. (Kimball A B, Jemec G B E, Alavi A, et al., Lancet 2023; 401(10378):747-61).

Bimekizumab (anti-IL-17A and anti-IL-17F) is another biologic agent approved by the FDA for the treatment of moderate to severe HS. But data from clinical studies BE HEARD I and BE HEARD II suggest that approximately 50% of patients did not benefit from IL-17A and IL-17F-directed inhibition with bimekizumab. (Kimball A B, Zouboulis C C, Sayed C, et. al., Bimekizumab in patients with moderate-to-severe hidradenitis suppurativa: 48-week efficacy and safety from BE HEARD I & II, two Phase III randomized, double-blind, placebo controlled, multicenter studies. Late-Breaking Platform Presentation at the 2023 American Academy of Dermatology Annual Meeting).

Berkemimab, a monoclonal antibody targeting IL-la, was studied in Phase II trials for the treatment of moderate to severe HS, but it failed to demonstrate efficacy in clinical trial no. NCT04019041. Another trial (NCT04988308) was prematurely terminated because the interim results met the prespecified futility criteria related to the primary endpoint (berkemimab was no better than placebo).

Other biologic therapies are being evaluated as potential treatments for hidradenitis suppurativa, including for example biologic therapies targeting IL-1α and/or IL-1β or their receptors (anakinra and lutikizumab and canakinumab), IL-1 and IL-18 (MAS825), IL12 and IL23 (ustekimumab), IL-17 (brodalumab and sonelokimab), and CD40 (iscalimab). More trials will be needed to determine the efficacy of these biologic therapies to treat hidradenitis suppurativa. (See Saunte D & Jemec G, JAMA, 2017; 318(20):2019-2032; Zouboulis C, et al., Exp. Dermatology, 2021; 30 (Suppl. 1): 8-17; Martora F, et al., Clinical, Cosmetic and Investigational Dermatol., 2023; 16, 134-148); Kanni, et al., J. Inv. Dermatology 2018; 138, 795-801); Clinicaltrials.gov ID NCT03827798).

Moreover, certain biologic therapies being evaluated for treatment of HS require daily injections, and continued injections may be required for treatment. For example, a randomized clinical trial of anakinra found that seven out of nine patients receiving daily injections of anakinra over a period of twelve weeks achieved a 50% reduction in HS severity (compared to 3 out of 10 for placebo). However, there was no significant difference between anakinra and placebo at a follow-up evaluation in week 24. (Saunte D & Jemec G, JAMA 2017; 318(20):2019-2032). Complicating the need for daily injections, anakinra has significant side effects, including injection site reactions in 71% of patients.

A recent meta-analysis and systematic review suggests that approximately half of patients with HS on a biologic will stop treatment by 12 months (Pham J P, Roseno N A L, J Am Acad Dermatol. 2024; 28:S0190-9622(24)00539-5). The frequency of incomplete and diminishing clinical responses for HS with existing therapies and the multifactorial pathogenesis of HS indicate that more treatment options are needed to ensure clinically meaningful and sustained disease control.

### SUMMARY OF THE INVENTION

The present disclosure includes, for example, methods of treating subjects having hidradenitis suppurativa comprising administering an effective amount of an anti-IL-1 antibody to a subject in need thereof.

IL-1β is expressed in a wide range of tissues and cells, including blood monocytes, lymphoid and non-lymphoid macrophages, and dendritic cells. IL-1β exerts strong proinflammatory activities at very low concentrations. Inactive IL-1β precursors accumulate in the cytosol until processed by NLRP3 inflammasome and caspase 1 into its active cytokine form (IL-1β). Inflammasome activation leads to rapid increases in active IL-1.

IL-1β is associated with the inflammatory cascade that leads to destruction of the pilosebaceous unit. For example, increased IL-1β levels have been observed in lesional skin. (Vossen ARJV, et al. J Invest Dermatol. 2020; 140(7):1463-1466.e2; Kelly G, et al. Br. J. Dermatol. 2015; 173(6):1431-1439). A clinical benefit has been observed in treating HS with anti-IL-1β biologic therapies, as discussed above, with anakinra, lutikizumab, canakinumab, and MAS825.

The invention is exemplified by the non-limiting embodiments below.

Embodiment 1. A method of treating hidradenitis suppurativa, comprising administering an effective amount of an anti-IL-1β antibody to a human subject in need thereof, wherein the anti-IL-1β antibody comprises:
i) (a) HCDR1 comprising the amino acid sequence of SEQ ID NO: 10; (b) HCDR2 comprising the amino acid sequence of SEQ ID NO: 21; (c) HCDR3 comprising the amino acid sequence of SEQ ID NO: 38; (d) LCDR1 comprising the amino acid sequence of SEQ ID NO: 39; (e) LCDR2 comprising the amino acid sequence of SEQ ID NO: 2; and (f) LCDR3 comprising the amino acid sequence of SEQ ID NO: 41; or
ii) (a) HCDR1 comprising the amino acid sequence of SEQ ID NO: 71; (b) HCDR2 comprising the amino acid sequence of SEQ ID NO: 72; (c) HCDR3 comprising the amino acid sequence of SEQ ID NO: 38; (d) LCDR1 comprising the amino acid sequence of SEQ ID NO: 39; (e) LCDR2 comprising the amino acid sequence of SEQ ID NO: 2; and (f) LCDR3 comprising the amino acid sequence of SEQ ID NO: 41; or
iii) (a) HCDR1 comprising the amino acid sequence of SEQ ID NO: 10; (b) HCDR2 comprising the amino acid sequence of SEQ ID NO: 21; (c) HCDR3 comprising the amino acid sequence of SEQ ID NO: 35; (d) LCDR1 comprising the amino acid sequence of SEQ ID NO: 37; (e) LCDR2 comprising the amino acid sequence of SEQ ID NO: 2; and (f) LCDR3 comprising the amino acid sequence of SEQ ID NO: 15; or
iv) (a) HCDR1 comprising the amino acid sequence of SEQ ID NO: 10; (b) HCDR2 comprising the amino acid sequence of SEQ ID NO: 16; (c) HCDR3 comprising the amino acid sequence of SEQ ID NO: 36; (d) LCDR1 comprising the amino acid sequence of SEQ ID NO: 39; (e) LCDR2 comprising the amino acid sequence of SEQ ID NO: 2; and (f) LCDR3 comprising the amino acid sequence of SEQ ID NO: 17; or
v) (a) HCDR1 comprising the amino acid sequence of SEQ ID NO: 10; (b) HCDR2 comprising the amino acid sequence of SEQ ID NO: 21; (c) HCDR3 comprising the amino acid sequence of SEQ ID NO: 35; (d) LCDR1 comprising the amino acid sequence of SEQ ID NO: 39; (e) LCDR2 comprising the amino acid sequence of SEQ ID NO: 2; and (f) LCDR3 comprising the amino acid sequence of SEQ ID NO: 17; or
vi) (a) HCDR1 comprising the amino acid sequence of SEQ ID NO: 10; (b) HCDR2 comprising the amino acid sequence of SEQ ID NO: 21; (c) HCDR3 comprising the amino acid sequence of SEQ ID NO: 30; (d) LCDR1 comprising the amino acid sequence of SEQ ID NO: 29; (e) LCDR2 comprising the amino acid sequence of SEQ ID NO: 2; and (f) LCDR3 comprising the amino acid sequence of SEQ ID NO: 17.
   Embodiment 2. The method of embodiment 1, wherein the anti-IL-1β antibody comprises:
i) (a) HCDR1 comprising the amino acid sequence of SEQ ID NO: 10; (b) HCDR2 comprising the amino acid sequence of SEQ ID NO: 21; (c) HCDR3 comprising the amino acid sequence of SEQ ID NO: 38; (d) LCDR1 comprising the amino acid sequence of SEQ ID NO: 39; (e) LCDR2 comprising the amino acid sequence of SEQ ID NO: 2; and (f) LCDR3 comprising the amino acid sequence of SEQ ID NO: 41; or
ii) (a) HCDR1 comprising the amino acid sequence of SEQ ID NO: 71; (b) HCDR2 comprising the amino acid sequence of SEQ ID NO: 72; (c) HCDR3 comprising the amino acid sequence of SEQ ID NO: 38; (d) LCDR1 comprising the amino acid sequence of SEQ ID NO: 39; (e) LCDR2 comprising the amino acid sequence of SEQ ID NO: 2; and (f) LCDR3 comprising the amino acid sequence of SEQ ID NO: 41.
   Embodiment 3. The method of embodiment 1 or embodiment 2, wherein the anti-IL-1β antibody comprises:
i) the CDRs of embodiment 1. i) and further comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO: 70; or
ii) the CDRS of claim 1. ii) and further comprises a VH comprising an amino acid sequence that is at least 90%, 91%, 92%, 93%, 94%, 95%, 96%, 97%, 98%, or 99% identical to the amino acid sequence of SEQ ID NO: 70.
   Embodiment 4. The method of any one of embodiments 1-3, wherein the anti-IL-1β antibody comprises:
i) the CDRs of embodiment 1. i) and further comprises a VH comprising the amino acid sequence of SEQ ID NO: 70; or
ii) the CDRS of embodiment 1. ii) and further comprises a VH comprising the amino acid sequence of SEQ ID NO: 70.
   Embodiment 5. The method of any one of embodiments 1-4, wherein the anti-IL-β antibody comprises:
i) the CDRs of embodiment 1. i) and further comprises a light chain variable region (VL) comprising an amino acid sequence that is at least 90%, 91%, 92%, 93%, 94%, 95%, 96%, 97%, 98%, or 99% identical to the amino acid sequence of SEQ ID NO: 69; or
ii) the CDRS of embodiment 1. ii) and further comprises a VL comprising an amino acid sequence that is at least 90%, 91%, 92%, 93%, 94%, 95%, 96%, 97%, 98%, or 99% identical to the amino acid sequence of SEQ ID NO: 69.
   Embodiment 6. The method of any one of embodiments 1-5, wherein the anti-IL-1β antibody comprises:
i) the CDRs of embodiment 1. i) and further comprises a VL comprising the amino acid sequence of SEQ ID NO: 69; or
ii) the CDRS of embodiment 1. ii) and further comprises a VL comprising the amino acid sequence of SEQ ID NO: 69.
   Embodiment 7. The method of any one of embodiments 1-6, wherein the anti-IL-1β antibody comprises a heavy chain variable region (VH) and a light chain variable region (VL), wherein:
the VH is at least 90%, 91%, 92%, 93%, 94%, 95%, 96%, 97%, 98%, 99%, or 100% identical to the amino acid sequence of SEQ ID NO: 70 and the VL is at least 90%, 91%, 92%, 93%, 94%, 95%, 96%, 97%, 98%, 99%, or 100% identical to the amino acid sequence of SEQ ID NO: 69.
   Embodiment 8. The method of any one of embodiments 1-7, wherein the anti-IL-1β antibody comprises a heavy chain variable region (VH) and a light chain variable region (VL), wherein the VH comprises the amino acid sequence of SEQ ID NO: 70 and the VL comprises the amino acid sequence of SEQ ID NO: 69.
   Embodiment 9. A method of treating hidradenitis suppurativa comprising administering an effective amount of an anti-IL-1β antibody to a human subject in need thereof, wherein the anti-IL-1β antibody thereof comprises a light chain variable region (VL) comprising an amino acid sequence that is at least 90%, 91%, 92%, 93%, 94%, 95%, 96%, 97%, 98%, or 99% identical to the amino acid sequence of SEQ ID NO: 69, and a heavy chain variable region (VH) comprising an amino acid sequence that is at least 90%, 91%, 92%, 93%, 94%, 95%, 96%, 97%, 98%, or 99% identical to the amino acid sequence of SEQ ID NO: 70.
   Embodiment 10. A method of treating hidradenitis suppurativa comprising administering an effective amount of an anti-IL-1β antibody to a human subject in need thereof, wherein the anti-IL-1β antibody thereof comprises a light chain variable region (VL) comprising the amino acid sequence of SEQ ID NO: 69, and a heavy chain variable region (VH) comprising the amino acid sequence of SEQ ID NO: 70.
   Embodiment 11. The method of embodiment 9, wherein the anti-IL-1β antibody comprises:
i) the VH and VL of embodiment 9 and (a) HCDR1 comprising the amino acid sequence of SEQ ID NO: 10; (b) HCDR2 comprising the amino acid sequence of SEQ ID NO: 21; (c) HCDR3 comprising the amino acid sequence of SEQ ID NO: 38; (d) LCDR1 comprising the amino acid sequence of SEQ ID NO: 39; (e) LCDR2 comprising the amino acid sequence of SEQ ID NO: 2; and (f) LCDR3 comprising the amino acid sequence of SEQ ID NO: 41; or
ii) the VH and VL of embodiment 9 and (a) HCDR1 comprising the amino acid sequence of SEQ ID NO: 71; (b) HCDR2 comprising the amino acid sequence of SEQ ID NO: 72; (c) HCDR3 comprising the amino acid sequence of SEQ ID NO: 38; (d) LCDR1 comprising the amino acid sequence of SEQ ID NO: 39; (e) LCDR2 comprising the amino acid sequence of SEQ ID NO: 2; and (f) LCDR3 comprising the amino acid sequence of SEQ ID NO: 41.
   Embodiment 12. The method of any one of embodiments 1-11, wherein the anti-IL-1β antibody is an antibody fragment.
   Embodiment 13. The method of embodiment 12, wherein the fragment is a Fab, Fab′, Fv, scFv or (Fab′)₂.
   Embodiment 14. The method of any one of embodiments 1-11, wherein the anti-IL-1β antibody is a full-length antibody.
   Embodiment 15. The method of any one of embodiments 1-11, wherein the anti-IL-13 antibody is an IgG antibody.
   Embodiment 16. The method of any one of embodiments 1-11, wherein an Fc region of the anti-IL-1β antibody comprises IgG1, IgG2, IgG3, or IgG4.
   Embodiment 17. The method of any one of embodiments 1-11, wherein the anti-IL-1β antibody comprises a human IgG1 heavy chain constant region.
   Embodiment 18. The method of any one of embodiments 1-11, wherein the anti-IL-1 antibody comprises a human IgG4 heavy chain constant region.
   Embodiment 19. The method of any one of embodiments 1-11 and 14-18, wherein the anti-IL-1β antibody comprises a human IgG kappa light chain constant region.
   Embodiment 20. The method of any one of embodiments 1-19, wherein the anti-IL-1β antibody is a humanized antibody.
   Embodiment 21. The method of any one of embodiments 1-11, wherein the anti-IL-β antibody comprises a light chain (LC) comprising an amino acid sequence having at least 90%, 91%, 92%, 93%, 94%, 95%, 96%, 97%, 98%, or 99% sequence identity to any one of SEQ ID NOs: 46, 48, 50, 52, and 54, and a heavy chain (HC) comprising an amino acid sequence having at least 90%, 91%, 92%, 93%, 94%, 95%, 96%, 97%, 98%, or 99% sequence identity to any one of SEQ ID NOs: 45, 49, 51, 53, 68, or 73.
   Embodiment 22. The method of any one of embodiments 1-11 or 21, wherein the anti-IL-1β antibody comprises:
(a) a light chain (LC) comprising an amino acid sequence having at least 85%, 90%, 91%, 92%, 93%, 94%, 95%, 96%, 97%, 98%, 99%, or 100% sequence identity to the amino acid sequence of SEQ ID NO: 48, and a heavy chain (HC) comprising an amino acid sequence having at least 85%, 90%, 91%, 92%, 93%, 94%, 95%, 96%, 97%, 98%, 99%, or 100% sequence identity to the amino acid sequence of SEQ ID NO: 68 or SEQ ID NO: 73; or
(b) a light chain (LC) comprising an amino acid sequence having at least 85%, 90%, 91%, 92%, 93%, 94%, 95%, 96%, 97%, 98%, or 99%, or 100% sequence identity to the amino acid sequence of SEQ ID NO: 46, and a heavy chain (HC) comprising an amino acid sequence having at least 85%, 90%, 91%, 92%, 93%, 94%, 95%, 96%, 97%, 98%, 99%, or 100% sequence identity to the amino acid sequence of SEQ ID NO: 45; or
(c) a light chain (LC) comprising an amino acid sequence having at least 85%, 90%, 91%, 92%, 93%, 94%, 95%, 96%, 97%, 98%, 99%, or 100% sequence identity to the amino acid sequence of SEQ ID NO: 50, and a heavy chain (HC) comprising an amino acid sequence having at least 85%, 90%, 91%, 92%, 93%, 94%, 95%, 96%, 97%, 98%, 99%, or 100% sequence identity to the amino acid sequence of SEQ ID NO: 49; or
(d) a light chain (LC) comprising an amino acid sequence having at least 85%, 90%, 91%, 92%, 93%, 94%, 95%, 96%, 97%, 98%, 99%, or 100% sequence identity to the amino acid sequence of SEQ ID NO: 52, and a heavy chain (HC) comprising an amino acid sequence having at least 85%, 90%, 91%, 92%, 93%, 94%, 95%, 96%, 97%, 98%, 99%, or 100% sequence identity to the amino acid sequence of SEQ ID NO: 51; or
(e) a light chain (LC) comprising an amino acid sequence having at least 85%, 90%, 91%, 92%, 93%, 94%, 95%, 96%, 97%, 98%, 99%, or 100% sequence identity to the amino acid sequence of SEQ ID NO: 54, and a heavy chain (HC) comprising an amino acid sequence having at least 85%, 90%, 91%, 92%, 93%, 94%, 95%, 96%, 97%, 98%, 99%, or 100% sequence identity to the amino acid sequence of SEQ ID NO: 53.
   Embodiment 23. The method of any one of embodiments 1-11, 21 or 22, wherein the anti-IL-1β antibody comprises a light chain (LC) comprising an amino acid sequence having at least 85%, 90%, 91%, 92%, 93%, 94%, 95%, 96%, 97%, 98%, 99%, or 100% sequence identity to the amino acid sequence of SEQ ID NO: 48, and a heavy chain (HC) sequence comprising an amino acid having at least 85%, 90%, 91%, 92%, 93%, 94%, 95%, 96%, 97%, 98%, 99%, or 100% sequence identity to the amino acid sequence of SEQ ID NO: 68 or SEQ ID NO: 73.
   Embodiment 24. The method of any one of embodiments 1-11 or 21-23, wherein the anti-IL-1β antibody comprises:
(a) a light chain comprising the amino acid sequence of SEQ ID NO: 48 and a heavy chain comprising the amino acid sequence of SEQ ID NO: 68 or SEQ ID NO: 73; or
(b) a light chain comprising the amino acid sequence of SEQ ID NO: 46 and a heavy chain comprising the amino acid sequence of SEQ ID NO: 45; or
(c) a light chain comprising the amino acid sequence of SEQ ID NO: 54 and a heavy chain comprising the amino acid sequence of SEQ ID NO: 53; or
(d) a light chain comprising the amino acid sequence of SEQ ID NO: 50 and a heavy chain comprising the amino acid sequence of SEQ ID NO: 49; or
(e) a light chain comprising the amino acid sequence of SEQ ID NO: 52 and a heavy chain comprising the amino acid sequence of SEQ ID NO: 51.
   Embodiment 25. The method of any one of embodiments 1-11 or 21-24, wherein the anti-IL-1β antibody comprises a light chain (LC) comprising the amino acid sequence of SEQ ID NO: 48 and a heavy chain (HC) comprising the amino acid sequence of SEQ ID NO: 68 or SEQ ID NO: 73.
   Embodiment 26. A method of treating hidradenitis suppurativa comprising administering an effective amount of an anti-IL-1β antibody to a human subject in need thereof, wherein the anti-IL-1β antibody thereof comprises a light chain (LC) comprising the amino acid sequence of SEQ ID NO: 48, and a heavy chain (HC) comprising the amino acid sequence of SEQ ID NO: 68 or SEQ ID NO: 73.
   Embodiment 27. The method of any one of embodiments 1-26, wherein the anti-IL-1β antibody is administered subcutaneously or intravenously.
   Embodiment 28. The method of any one of embodiments 1-27, wherein a loading dose of 600 mg of the anti-IL-1β antibody is administered followed by a dose of 300 mg of the anti-IL-1β antibody every four weeks (Q4W), optionally, wherein the first 300 mg dose is administered four weeks after the loading dose.
   Embodiment 29. The method of any one of embodiments 1-27, wherein a loading dose of 300 mg of the anti-IL-1β antibody is administered followed by a dose of 150 mg of the anti-IL-1β antibody every two weeks (Q2W), optionally, wherein the first 150 mg dose is administered two weeks after the loading dose.
   Embodiment 30. The method of any one of embodiments 1-29, wherein the subject has moderate to severe hidradenitis suppurativa.
   Embodiment 31. The method of any one of embodiments 1-30, wherein the subject is an adult.
   Embodiment 32. The method of any one of embodiments 1-31, wherein administration of the anti-IL-1β antibody results in a decrease in abscess and inflammatory nodule (AN) count in the subject, compared to baseline.
   Embodiment 33. The method of any one of embodiments 1-31, wherein administration of the anti-IL-1β antibody results in no increase from baseline in abscess count, draining fistula, and/or AN count in the subject, compared to baseline.
   Embodiment 34. The method of any one of embodiments 1-31, wherein administration of the anti-IL-1β antibody results in a decrease in draining fistula count in the subject, compared to baseline.
   Embodiment 35. The method of any one of embodiments 1-31, wherein administration of the anti-IL-1β antibody results in a decrease in the subject's International Hidradenitis Suppurativa Severity Score System (IHS4) score, compared to baseline, optionally, wherein administration of the anti-IL-1β antibody results in a reduction in IHS4 score from severe disease to moderate disease or from moderate disease to mild disease.
   Embodiment 36. The method of any one of embodiments 1-31, wherein the subject achieves HiSCR50, HiSCR75, HiSCR90, and/or HiSCR100, optionally, wherein the subject achieves HiSCR50, HiSCR75, HiSCR90, and/or HiSCR100 at or after 16 weeks from the start of anti-IL-1β antibody treatment.
   Embodiment 37. The method of any one of embodiments 1-31, wherein the subject has a NRS≥3, optionally, at baseline.
   Embodiment 38. The method of embodiment 37, wherein the subject achieves NRS30.
   Embodiment 39. The method of any one of embodiments 1-31, wherein the subject does not have anti-drug antibodies to the anti-IL-1β antibody, optionally, at or after 16 weeks after the start of anti-IL-1β antibody treatment.
   Embodiment 40. The method of any one of embodiments 1-31, wherein administration of the anti-IL-1β antibody reduces hidradenitis suppurativa flares in the subject, as compared to baseline.
   Embodiment 41. The method of any one of embodiments 1-31, wherein administration of the anti-IL-1β antibody increases the time between hidradenitis suppurativa flares in the subject.
   Embodiment 42. The method of any one of embodiments 1-41, wherein: (i) the subject has failed treatment with an anti-TNF therapy; (ii) anti-TNF therapy is contraindicated for the subject; or (iii) the subject has not failed treatment with an anti-TNF therapy.
   Embodiment 43. The method of any one of embodiments 1-31 and 42, wherein the anti-IL-1 antibody is administered to a population of human subjects in need thereof, and wherein at least 10-15%, 15%-20%, 20%-25%, 25%-30%, 30%-35%, 35%-40%, 40%-45%, 45%-50%, 50%-55%, 55%-60%, 60%-65%, 65%-70%, 70%-75%, 75%-80%, 80%-85%, 85%-90%, 90%-95%, and/or 95%-100% of the population of human subjects achieve HiSCR50, HiSCR75, HiSCR90, and/or HiSCR100 at or after 16 weeks from the start of anti-IL-1β antibody treatment.
   Embodiment 44. The method of any one of embodiments 1-31 and 42, wherein the anti-IL-1 antibody is administered to a population of human subjects in need thereof, and wherein 10% of the population of human subjects experienced a hidradenitis suppurativa flare during the time period between administration of the loading dose and 16 weeks after administration of the loading dose.

### BRIEF DESCRIPTION OF THE DRAWINGS

FIG. 1 shows a clinical study design for treating patients with moderate to severe HS, with “Antibody A.” “Antibody A” refers to an anti-IL-1β antibody, wherein the anti-IL-1β antibody comprises the following six CDRs: a heavy chain CDR1 having an amino acid sequence of SEQ ID NO: 71; a heavy chain CDR2 having an amino acid sequence of SEQ ID NO: 72; a heavy chain CDR3 having an amino acid sequence of SEQ ID NO: 38; a light chain CDR1 having an amino acid sequence of SEQ ID NO: 39; a light chain CDR2 having an amino acid sequence of SEQ ID NO: 2; and a light chain CDR3 having an amino acid sequence of SEQ ID NO: 41. Antibody A has a variable heavy chain region (VH) having an amino acid sequence of SEQ ID NO: 70 and a variable light chain region (VL) having an amino acid sequence of SEQ ID NO: 69. Antibody A has a heavy chain having an amino acid sequence of SEQ ID NO: 68 (or SEQ ID NO: 73, which is SEQ ID NO: 68 without a leader sequence) and a light chain having an amino acid sequence of SEQ ID NO: 48. “Q2W” refers to every 2 weeks. “Q4W” refers to every 4 weeks. “EOT” refers to End of Treatment. “EOS” refers to End of Study.

FIG. 2 is a table of reference ranges showing results considered normal for high sensitivity C-reactive protein (hs-CRP).

### DETAILED DESCRIPTION OF THE INVENTION

The following definitions are provided to facilitate an understanding of the invention. They are not intended to limit the invention in any way.

### Definitions

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. As used herein, the term refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)₂. The term antibody also includes, but is not limited to, chimeric antibodies, humanized antibodies, human antibodies, and antibodies of various species such as mouse, cynomolgus monkey, etc.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “heavy chain variable region” refers to a region comprising a heavy chain complementary determining region (CDR) 1, framework region (FR) 2, CDR2, FR3, and CDR3 of the heavy chain. In some embodiments, a heavy chain variable region also comprises at least a portion of an FR1 and/or at least a portion of an FR4. In some embodiments, a heavy chain CDR1 corresponds to Kabat residues 31 to 35; a heavy chain CDR2 corresponds to Kabat residues 50 to 65; and a heavy chain CDR3 corresponds to Kabat residues 95 to 102. (See, e.g., Kabat Sequences of Proteins of Immunological Interest (1987 and 1991, NIH, Bethesda, Md.)).

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence. The term “light chain variable region” refers to a region comprising a light chain CDR1, FR2, HVR2, FR3, and HVR3. In some embodiments, a light chain variable region also comprises an FR1 and/or an FR4. In some embodiments, a light chain CDR1 corresponds to Kabat residues 24 to 34; a light chain CDR2 corresponds to Kabat residues 50 to 56; and a light chain CDR3 corresponds to Kabat residues 89 to 97. (See, e.g., Kabat Sequences of Proteins of Immunological Interest (1987 and 1991, NIH, Bethesda, Md.)).

A “humanized antibody” refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, an scFv, a (Fab′)₂, etc.

The term “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The terms “inhibition” or “inhibit” refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, “reduce” or “inhibit” means the ability to cause an overall decrease of 20% or greater. In another embodiment, “reduce” or “inhibit” means the ability to cause an overall decrease of 50% or greater. In yet another embodiment, by “reduce” or “inhibit” means the ability to cause an overall decrease of 75%, 85%, 90%, 95%, or greater.

“Sample” or “subject sample” or “biological sample” generally refers to a sample which may be tested for a particular molecule. Samples may include but are not limited to cells, body fluids, including blood, serum, plasma, urine, saliva, stool, tears, pleural fluid and the like.

“Baseline” as used herein refers to a time period of approximately one week before the anti-IL-1β antibody is first administered to the subject.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the SNP containing nucleic acids described herein or their encoded proteins. Agents are evaluated for potential biological activity by inclusion in screening assays.

As used herein, “moderate to severe hidradenitis suppurativa” is defined as a total of at least 5 inflammatory lesions, i.e., an AN count of ≥5 and hidradenitis suppurativa inflammatory lesions in at least 2 distinct anatomic areas, at least one of which is Hurley Stage 2 or 3.

As used herein, “fistula” refers to a pathologic passageway connecting to the skin surface from dermis or subcutaneous tissue. Fistulas are also referred to as “tunnels” or sinus tracts. Fistulas can be draining or non-draining. Draining fistulas drain serous or purulent fluid, either spontaneously or by gentle palpation. Non-draining fistulas do not drain serous or purulent fluid, either spontaneously or by gentle palpation. As used herein, “anal fistula” in hidradenitis suppurativa are described as abnormal tunnels or tracts that connect the anal canal to the perianal skin, often originating from a pit-like scar within the anal canal, and can appear as a visible opening on the skin with potential drainage of pus, sometimes accompanied by pain, swelling, and redness in the affected area. These fistulas can be classified based on their depth and trajectory through the anal sphincter muscles depending on the severity of the condition.

As used herein, a subject who has TNF failure, also referred to as a subject who has failed treatment with an anti-TNF therapy, is defined as a subject with lack of response or loss of response to an anti-TNF therapy and is not intolerant to anti-TNF therapy.

As used herein, a subject who has not failed treatment with an anti-TNF therapy as used herein is defined as a subject who is anti-TNF naïve or is a subject who has been exposed to anti-TNF therapy and has not failed.

As used herein, a “hidradenitis suppurativa flare” or “HS flare” is defined as ≥25% increase in AN count plus an increase of ≥2 in AN count compared to baseline.

A “subject” can be mammalian. In any of the embodiments involving a subject, the subject can be human. In any of the embodiments involving a subject, the subject can be a cow, pig, monkey, sheep, dog, cat, fish, or poultry.

A “pediatric” subject herein is a human of less than 18 years of age, whereas an “adult” subject is 18 years or older.

As used herein, “treatment” or “treat” refers to any administration or application of a therapeutic for disease or disorder in a subject, and includes inhibiting the disease, arresting its development, relieving one or more symptoms of the disease, or curing the disease.

As used herein, “prevention” or “prevent” refers to prophylaxis, avoidance of disease manifestation, a delay of onset, and/or reduction in frequency and/or severity of one or more symptoms of a particular disease, disorder, or condition (e.g., HS). In some embodiments, an agent is considered to “prevent” a particular disease, disorder, or condition if there is a decrease in the development, frequency, and/or intensity of one or more symptoms of the disease, disorder, or condition after administration of an agent.

The term “effective amount” or “therapeutically effective amount” refers to an amount of an active ingredient effective for treatment or prevention of a disease or disorder in a subject, such as to partially or fully relieve one or more symptoms. In some embodiments, an effective amount refers to an amount effective, at dosages and for periods of time necessary, to achieve the desired therapeutic or prophylactic result.

“Improved” herein is used to indicate that a HS-associated parameter is quantified at a baseline time point and at a time point after administration of the anti-IL-1β antibody. The difference between the value of the parameter at a particular time point following initiation of treatment and the value of the parameter at a baseline time point is used to establish whether there has been an “improvement” in the HS-associated parameter. This can be an increase or decrease, depending on the specific parameter measured.**

Methods of Treating Hidradenitis Suppurativa with Anti-IL-1β Antibodies

In some embodiments, a method of treating a subject having HS is provided comprising administering an anti-IL-1β antibody to a subject in need thereof.

HS has been classified using the Hurley Stages of HS. A Hurley Stage may be determined for each HS-affected anatomical region. There are three stages. Stage I may be defined as abscess formation (single or multiple) without sinus tracts and cicatrization. Stage II may be defined as recurrent abscesses with tract formation and cicatrization, and single or multiple widely separated lesions. Stage III may be defined as diffuse, or near-diffuse, involvement or multiple interconnected tracts and abscesses across the entire diseased area.

In some embodiments, the subject has moderate to severe hidradenitis suppurativa. In some embodiments the subject has at least one lesion that is Hurley Stage 2. In some embodiments, the subject has at least one lesion that is Hurley Stage 3.

The severity of HS has been evaluated using various methodologies, including the International Hidradenitis Suppurativa Severity Score System (IHS4), the modified Sartorius Score (mSS), and the HS-Physician's Global Assessment (HS-PGA), all of which are known to those of ordinary skill in the art. The IHS4 system assigns a weighted score to lesions by categorizing them as nodules, abscesses, or draining tunnels. The IHS4 score is arrived at by the number of nodules (multiplied by 1) plus the number of abscesses (multiplied by 2) plus the number of draining tunnels (multiplied by 4). A total score of 3 or less signifies mild disease, 4 to 10 signifies moderate disease, and 11 or higher signifies severe disease. The mSS is determined by counting, classifying, and measuring distance between lesions in a predetermined region. A HS-PGA score is determined based on an objective count of total HS lesions.

In some embodiments, the subject has mild HS according to the IHS4 system. In some embodiments, the subject has moderate HS according to the IHS4 system. In some embodiments, the subject has severe HS according to the IHS4 system.

The Hidradenitis Suppurativa Clinical Response (HiSCR) is a measure of how patients respond to clinical treatment. The HiSCR considers the total number of inflammatory lesions, including inflammatory nodules and abscesses (also referred to as AN count). A 50% reduction from baseline in the total abscess and inflammatory nodule (AN) count, with no increase from baseline in abscess count or draining fistula count, results in a HiSCR50 score. A 75% reduction from baseline in the total abscess and inflammatory nodule (AN) count, with no increase from baseline in abscess count or draining fistula count, results in a HiSCR75 score. A 90% reduction from baseline in the total abscess and inflammatory nodule (AN) count, with no increase from baseline in abscess count or draining fistula count, results in a HiSCR90 score. A 100% reduction from baseline in the total abscess and inflammatory nodule (AN) count, with no increase from baseline in abscess count or draining fistula count, results in a HiSCR100 score.

NRS30 is another measure of how patients respond to clinical treatment. NRS30 is defined as at least a 30% reduction and/or at least a 1-unit reduction on a Numerical Rating Scale (NRS) in Patient's Global Assessment of Skin Pain (PGA Skin Pain) score.

In some embodiments, administration of the anti-IL-1β antibody results in no increase from baseline in abscess count, draining fistula, and/or AN count in the subject. In some embodiments, administration of the anti-IL-1β antibody results in no increase from baseline in abscess count, draining fistula, and/or AN count in the subject compared to baseline.

In some embodiments, the subject has had signs and symptoms of HS for at least 6 months. In some embodiments, the subject has had signs and symptoms of HS for at least 3 months. In some embodiments, the subject has had signs and symptoms of HS for at least 9 months. In some embodiments, the subject has had signs and symptoms of HS for at least one year. In some embodiments, the subject has had signs and symptoms of HS for at least two years.

In some embodiments, administration of the anti-IL-1β antibody results in a decrease in AN count in the subject. As used herein, “AN count” and “total AN count” are equivalent in meaning. In some embodiments, the decrease is compared to baseline. In some embodiments, the decrease is a 10%, 20%, 25%, 30%, 40%, 50%, 60%, 70%, 80%, 90%, or 100% decrease. In some embodiments, after administration of the anti-IL-13 antibody, the subject has an AN count of 20 or less. In some embodiments, after administration of the anti-IL-1β antibody, the subject has an AN count of 15 or less. In some embodiments, after administration of the anti-IL-1β antibody, the subject has an AN count of 10 or less. In some embodiments, after administration of the anti-IL-1β antibody, the subject has an AN count of 9 or less. In some embodiments, after administration of the anti-IL-1β antibody, the subject has an AN count of 8 or less. In some embodiments, after administration of the anti-IL-1β antibody, the subject has an AN count of 7 or less. In some embodiments, after administration of the anti-IL-1β antibody, the subject has an AN count of 6 or less. In some embodiments, after administration of the anti-IL-1β antibody, the subject has an AN count of 5 or less. In some embodiments, after administration of the anti-IL-1β antibody, the subject has an AN count of 4 or less. In some embodiments, after administration of the anti-IL-1β antibody, the subject has an AN count of 3 or less. In some embodiments, after administration of the anti-IL-1β antibody, the subject has an AN count of 2 or less. In some embodiments, after administration of the anti-IL-1β antibody, the subject has an AN count of 1 or less.

In some embodiments, administration of the anti-IL-1β antibody results in a decrease in draining fistula count in the subject. In some embodiments, the decrease is compared to baseline. In some embodiments, the decrease is a 10%, 20%, 25%, 30%, 40%, 50%, 60%, 70%, 80%, 90%, or 100% decrease. In some embodiments, after administration of the anti-IL-1β antibody, the subject has a draining fistula count of 20 or less. In some embodiments, after administration of the anti-IL-1β antibody, the subject has a draining fistula count of 15 or less. In some embodiments, after administration of the anti-IL-1β antibody, the subject has a draining fistula count of 10 or less. In some embodiments, after administration of the anti-IL-1β antibody, the subject has a draining fistula count of 9 or less. In some embodiments, after administration of the anti-IL-1β antibody, the subject has a draining fistula count of 8 or less. In some embodiments, after administration of the anti-IL-1β antibody, the subject has a draining fistula count of 7 or less. In some embodiments, after administration of the anti-IL-1β antibody, the subject has a draining fistula count of 6 or less. In some embodiments, after administration of the anti-IL-1β antibody, the subject has a draining fistula count of 5 or less. In some embodiments, after administration of the anti-IL-1β antibody, the subject has a draining fistula count of 4 or less. In some embodiments, after administration of the anti-IL-1β antibody, the subject has a draining fistula count of 3 or less. In some embodiments, after administration of the anti-IL-1β antibody, the subject has a draining fistula count of 2 or less. In some embodiments, after administration of the anti-IL-1β antibody, the subject has a draining fistula count of 1 or less.

In some embodiments, the anti-IL-1β antibody is administered subcutaneously. In some embodiments, the anti-IL-1β antibody is administered intravenously.

In some embodiments, the anti-IL-1β antibody is administered at a dose of 150 mg. In some embodiments, the anti-IL-1β antibody is administered at a dose of 300 mg. In some embodiments, the anti-IL-1β antibody is administered at a dose of 300 mg every four weeks (Q4W). In some embodiments, the anti-IL-1β antibody is administered at a dose of 150 mg every two weeks (Q2W). In some embodiments, the anti-IL-1β antibody is administered for 16 weeks. In some embodiments, the anti-IL-1β antibody is administered for 14 weeks. In some embodiments, the anti-IL-1β antibody is administered for 12 weeks. In some embodiments, the anti-IL-1β antibody is administered for 24 weeks. In some embodiments, the anti-IL-1β antibody is administered for 22 weeks. In some embodiments, the anti-IL-1β antibody is administered for 20 weeks. In some embodiments, the anti-IL-1β antibody is administered for seven months, i.e. 16 weeks followed by three months. In some embodiments, a loading dose of 600 mg of the anti-IL-1β antibody is administered. In some embodiments, a loading dose of 300 mg of the anti-IL-1β antibody is administered. In some embodiments, a loading dose of 600 mg of the anti-IL-1β antibody is administered followed by a dose of 300 mg every four weeks (Q4W). In some embodiments, the first 300 mg dose is administered four weeks after the loading dose. In some embodiments, a loading dose of 300 mg of the anti-IL-1β antibody is administered followed by a dose of 150 mg every two weeks (Q2W). In some embodiments, the first 150 mg dose is administered two weeks after the loading dose. In some embodiments, the first dose that is not a loading dose (i.e., a maintenance dose) is administered two weeks after the loading dose. In some embodiments, the first dose that is not a loading dose (i.e., a maintenance dose) is administered four weeks after the loading dose. In some embodiments, a loading dose of 600 mg of the anti-IL-1β antibody is administered subcutaneously followed by a dose of 300 mg every four weeks (Q4W), also administered subcutaneously. In some embodiments, a loading dose of 300 mg of the anti-IL-1β antibody is administered subcutaneously followed by a dose of 150 mg every two weeks (Q2W), also administered subcutaneously.

In some embodiments, the subject is an adult. In some embodiments, the subject is post-pubescent. In some embodiments, the subject is a pediatric subject. In some embodiments, the subject has high IL-6 levels in blood. In some embodiments, the subject has healthy IL-6 levels in blood. In some embodiments, IL-6 levels of between 0 and 43.5 pg/ml in blood may be considered healthy IL-6 levels. See Said, et al., Defining IL-6 Levels in Healthy Individuals: A Meta-Analysis, J. Med. Virology, 93(6): 3915-24 (2021). In some embodiments, IL-6 levels higher than 40 μg/ml in blood may be considered high IL-6 levels. See Alende-Castro, et al., Serum Concentrations of Interleukin 6 in the General Adult Population: Possible Implications for Anti-IL-6 Therapy in SARS-Cov-2 Infections and IL-6-Related Diseases, J. Investig. Allergol. Clin. Immunol., 31(1): 75-78 (2021).

In some embodiments, administration of the anti-IL-1β antibody reduces the level of high sensitivity C-reactive Protein (hs-CRP) in the subject. Reductions are as compared to baseline. In some embodiments, the subject has healthy IL-6 levels after treatment with the anti-IL-1β antibody. In some embodiments, administration of the anti-IL-1β antibody reduces inflammation.

In some embodiments, administration of the anti-IL-1β antibody reduces inflammation caused by HS. In some embodiments, administration of the anti-IL-1β antibody decreases the total count of inflammatory lesions caused by HS. In some embodiments, administration of the anti-IL-1β antibody decreases the total count of inflammatory lesions by 50% or more. In some embodiments, administration of the anti-IL-1β antibody is associated with the absence of new abscess formation. In some embodiments, administration of the anti-IL-1β antibody provides decreased neovascularization and lesion skin depth.

In some embodiments, administration of the anti-IL-1β antibody results in an improved IHS4 score. In some embodiments, administration of the anti-IL-1β antibody results in a decrease in the subject's IHS4 score. In some embodiments, the decrease in IHS4 score is compared to baseline. In some embodiments, administration of the anti-IL-1β antibody results in a reduction in IHS4 score from severe disease to moderate disease. In some embodiments, administration of the anti-IL-1β antibody results in a reduction in IHS4 score from moderate disease to mild disease. In some embodiments, the decrease or reduction in IHS4 score is compared to baseline. In some embodiments, administration of the anti-IL-1β antibody results in an improved mSS. In some embodiments, administration of the anti-IL-1β antibody results in an improved HS-PGA.

In some embodiments, administration of the anti-IL-1β antibody results in an improved Hidradenitis Suppurativa Quality of Life Instrument (HiSQOL™) score. In some embodiments, improvements are compared to baseline. The total score for the HiSQOL™ has range from 0 to 68, with a higher score indicating a higher level of symptomology. In some embodiments, administration of the anti-IL-1β antibody results in an improvement in HiSQOL™ score by at least 5 points, 10 points, 15 points, 20 points, 25 points, 30 points, 35 points, 40 points, 45 points, 50 points, 55 points, 60 points, or 65 points. In some embodiments, the improvement is an average score improvement, optionally in a population of subjects.

In some embodiments, administration of the anti-IL-1β antibody results in an improved Dermatology Life Quality Index (DLQI) score. In some embodiments, the improvement is compared to baseline. The DLQI has a maximum score of 30 and a minimum score of 0. A score higher than 10 indicates that the subject's life is being severely affected by their skin disease. In some embodiments, administration of the anti-IL-1β antibody results in an improvement in DLQI score by at least 2 points, 4 points, 6 points, 8 points, 10 points, 12 points, 14 points, 16 points, 18 points, 20 points, 22 points, 24 points, 26 points, 28 points, or 30 points. In some embodiments, the improvement is an average score improvement, optionally in a population of subjects.

In some embodiments, administration of the anti-IL-1β antibody results in an improved Patient Health Questionnaire (PHQ-9) score. In some embodiments, the improvement is compared to baseline. The total sum of the responses in the PHQ-9 roughly indexes levels of depression. Scores range from 0 to 27. In general, a total of 10 or above is suggestive of the presence of depression. In some embodiments, administration of the anti-IL-1β antibody results in an improvement in PHQ-9 score by at least 2 points, 4 points, 6 points, 8 points, 10 points, 12 points, 14 points, 16 points, 18 points, 20 points, 22 points, 24 points, 26 points, or 27 points. In some embodiments, the improvement is an average score improvement, optionally in a population of subjects.

In some embodiments, administration of the anti-IL-1β antibody results in an improvement in the PGA of Skin Pain score. The PGA of Skin Pain score is assessed based on a scale of 10, with 0 meaning no skin pain and 10 meaning skin pain as bad as you can imagine. In some embodiments, improvements are compared to baseline. In some embodiments, the subject's PGA of Skin Pain score decreases by 1, 2, 3, 4, 5, 6, 7, 8, 9, or 10. In some embodiments, the improvement is an average score improvement, optionally in a population of subjects.

In some embodiments, administration of the anti-IL-1β antibody results in at least a 30% reduction and at least a 1-unit reduction from baseline on a Numerical Rating Scale (NRS) in Patient's Global Assessment of Skin Pain (PGA Skin Pain) in a subject, also known as NRS30. In some embodiments, the subject has a Baseline NRS in PGA Skin Pain ≥3.

In some embodiments, administration of the anti-IL-1β antibody provides an improved clinical outcome determined by the HiSCR score. In some embodiments, the subject achieves HiSCR50, HiSCR75, HiSCR90, and/or HiSCR100. In some embodiments, the subject achieves the HiSCR50, HiSCR75, HiSCR90, and/or HiSCR100 score at or after 2 weeks, 4 weeks, 6 weeks, 8 weeks, 10 weeks, 12 weeks, 14 weeks, 16 weeks, 18 weeks, 20 weeks, 22 weeks, 24 weeks, 26 weeks, 28 weeks, 30 weeks, 32 weeks, 34 weeks, or 36 weeks from the start of anti-IL-1β antibody treatment. In some embodiments, a subject administered the anti-IL-1β antibody achieves HiSCR75 at or after 16 weeks. In some embodiments, a subject administered the anti-IL-1β antibody achieves HiSCR50 at or after 16 weeks. In some embodiments, a subject administered the anti-IL-1β antibody achieves HiSCR90 at or after 16 weeks. In some embodiments, a subject administered the anti-IL-1β antibody achieves HiSCR100 at or after 16 weeks. In some embodiments, a subject administered the anti-IL-1β antibody achieves HiSCR75. In some embodiments, a subject administered the anti-IL-1β antibody achieves HiSCR50. In some embodiments, a subject administered the anti-IL-1β antibody achieves HiSCR90. In some embodiments, a subject administered the anti-IL-1β antibody achieves HiSCR100.

In some embodiments, the anti-IL-1β antibody is administered to a population of human subjects in need thereof. In some embodiments, 10-15% of the population of human subjects achieve HiSCR50 at or after 16 weeks from the start of anti-IL-1β antibody treatment. In some embodiments, 15%-20% of the population of human subjects achieve HiSCR50 at or after 16 weeks from the start of anti-IL-1β antibody treatment. In some embodiments, 20%-25% of the population of human subjects achieve HiSCR50 at or after 16 weeks from the start of anti-IL-1β antibody treatment. In some embodiments, 25%-30% of the population of human subjects achieve HiSCR50 at or after 16 weeks from the start of anti-IL-1β antibody treatment. In some embodiments, 30%-35% of the population of human subjects achieve HiSCR50 at or after 16 weeks from the start of anti-IL-1β antibody treatment. In some embodiments, 35%-40% of the population of human subjects achieve HiSCR50 at or after 16 weeks from the start of anti-IL-1β antibody treatment. In some embodiments, 40%-45% of the population of human subjects achieve HiSCR50 at or after 16 weeks from the start of anti-IL-1β antibody treatment. In some embodiments, 45%-50% of the population of human subjects achieve HiSCR50 at or after 16 weeks from the start of anti-IL-1β antibody treatment. In some embodiments, 50%-55% of the population of human subjects achieve HiSCR50 at or after 16 weeks from the start of anti-IL-1β antibody treatment. In some embodiments, 55%-60% of the population of human subjects achieve HiSCR50 at or after 16 weeks from the start of anti-IL-1β antibody treatment. In some embodiments, 60%-65% of the population of human subjects achieve HiSCR50 at or after 16 weeks from the start of anti-IL-1β antibody treatment. In some embodiments, 65%-70% of the population of human subjects achieve HiSCR50 at or after 16 weeks from the start of anti-IL-1β antibody treatment. In some embodiments, 70%-75% of the population of human subjects achieve HiSCR50 at or after 16 weeks from the start of anti-IL-1β antibody treatment. In some embodiments, 75%-80% of the population of human subjects achieve HiSCR50 at or after 16 weeks from the start of anti-IL-1β antibody treatment. In some embodiments, 80%-85% of the population of human subjects achieve HiSCR50 at or after 16 weeks from the start of anti-IL-1β antibody treatment. In some embodiments, 85%-90% of the population of human subjects achieve HiSCR50 at or after 16 weeks from the start of anti-IL-1β antibody treatment. In some embodiments, 90%-95% of the population of human subjects achieve HiSCR50 at or after 16 weeks from the start of anti-IL-1β antibody treatment. In some embodiments, 95%-100% of the population of human subjects achieve HiSCR50 at or after 16 weeks from the start of anti-IL-1β antibody treatment. In some embodiments, 10-15% of the population of human subjects achieve HiSCR75 at or after 16 weeks from the start of anti-IL-1β antibody treatment. In some embodiments, 15%-20% of the population of human subjects achieve HiSCR75 at or after 16 weeks from the start of anti-IL-1β antibody treatment. In some embodiments, 20%-25% of the population of human subjects achieve HiSCR75 at or after 16 weeks from the start of anti-IL-1β antibody treatment. In some embodiments, 25%-30% of the population of human subjects achieve HiSCR75 at or after 16 weeks from the start of anti-IL-1β antibody treatment. In some embodiments, 30%-35% of the population of human subjects achieve HiSCR75 at or after 16 weeks from the start of anti-IL-1β antibody treatment. In some embodiments, 35%-40% of the population of human subjects achieve HiSCR75 at or after 16 weeks from the start of anti-IL-1β antibody treatment. In some embodiments, 40%-45% of the population of human subjects achieve HiSCR75 at or after 16 weeks from the start of anti-IL-1β antibody treatment. In some embodiments, 45%-50% of the population of human subjects achieve HiSCR75 at or after 16 weeks from the start of anti-IL-1β antibody treatment. In some embodiments, 50%-55% of the population of human subjects achieve HiSCR75 at or after 16 weeks from the start of anti-IL-1β antibody treatment. In some embodiments, 55%-60% of the population of human subjects achieve HiSCR75 at or after 16 weeks from the start of anti-IL-1β antibody treatment. In some embodiments, 60%-65% of the population of human subjects achieve HiSCR75 at or after 16 weeks from the start of anti-IL-1β antibody treatment. In some embodiments, 65%-70% of the population of human subjects achieve HiSCR75 at or after 16 weeks from the start of anti-IL-1β antibody treatment. In some embodiments, 70%-75% of the population of human subjects achieve HiSCR75 at or after 16 weeks from the start of anti-IL-1β antibody treatment. In some embodiments, 75%-80% of the population of human subjects achieve HiSCR75 at or after 16 weeks from the start of anti-IL-1β antibody treatment. In some embodiments, 80%-85% of the population of human subjects achieve HiSCR75 at or after 16 weeks from the start of anti-IL-1β antibody treatment. In some embodiments, 85%-90% of the population of human subjects achieve HiSCR75 at or after 16 weeks from the start of anti-IL-1β antibody treatment. In some embodiments, 90%-95% of the population of human subjects achieve HiSCR75 at or after 16 weeks from the start of anti-IL-1β antibody treatment. In some embodiments, 95%-100% of the population of human subjects achieve HiSCR75 at or after 16 weeks from the start of anti-IL-13 antibody treatment. In some embodiments, 10-15% of the population of human subjects achieve HiSCR90 at or after 16 weeks from the start of anti-IL-1β antibody treatment. In some embodiments, 15%-20% of the population of human subjects achieve HiSCR90 at or after 16 weeks from the start of anti-IL-1β antibody treatment. In some embodiments, 20%-25% of the population of human subjects achieve HiSCR90 at or after 16 weeks from the start of anti-IL-1β antibody treatment. In some embodiments, 25%-30% of the population of human subjects achieve HiSCR90 at or after 16 weeks from the start of anti-IL-1β antibody treatment. In some embodiments, 30%-35% of the population of human subjects achieve HiSCR90 at or after 16 weeks from the start of anti-IL-1β antibody treatment. In some embodiments, 35%-40% of the population of human subjects achieve HiSCR90 at or after 16 weeks from the start of anti-IL-1β antibody treatment. In some embodiments, 40%-45% of the population of human subjects achieve HiSCR90 at or after 16 weeks from the start of anti-IL-1β antibody treatment. In some embodiments, 45%-50% of the population of human subjects achieve HiSCR90 at or after 16 weeks from the start of anti-IL-1β antibody treatment. In some embodiments, 50%-55% of the population of human subjects achieve HiSCR90 at or after 16 weeks from the start of anti-IL-1β antibody treatment. In some embodiments, 55%-60% of the population of human subjects achieve HiSCR90 at or after 16 weeks from the start of anti-IL-1β antibody treatment. In some embodiments, 60%-65% of the population of human subjects achieve HiSCR90 at or after 16 weeks from the start of anti-IL-1β antibody treatment. In some embodiments, 65%-70% of the population of human subjects achieve HiSCR90 at or after 16 weeks from the start of anti-IL-1β antibody treatment. In some embodiments, 70%-75% of the population of human subjects achieve HiSCR90 at or after 16 weeks from the start of anti-IL-1β antibody treatment. In some embodiments, 75%-80% of the population of human subjects achieve HiSCR90 at or after 16 weeks from the start of anti-IL-1β antibody treatment. In some embodiments, 80%-85% of the population of human subjects achieve HiSCR90 at or after 16 weeks from the start of anti-IL-1β antibody treatment. In some embodiments, 85%-90% of the population of human subjects achieve HiSCR90 at or after 16 weeks from the start of anti-IL-1β antibody treatment. In some embodiments, 90%-95% of the population of human subjects achieve HiSCR90 at or after 16 weeks from the start of anti-IL-1β antibody treatment. In some embodiments, 95%-100% of the population of human subjects achieve HiSCR90 at or after 16 weeks from the start of anti-IL-1β antibody treatment. In some embodiments, 10-15% of the population of human subjects achieve HiSCR100 at or after 16 weeks from the start of anti-IL-1β antibody treatment. In some embodiments, 15%-20% of the population of human subjects achieve HiSCR100 at or after 16 weeks from the start of anti-IL-1β antibody treatment. In some embodiments, 20%-25% of the population of human subjects achieve HiSCR100 at or after 16 weeks from the start of anti-IL-1β antibody treatment. In some embodiments, 25%-30% of the population of human subjects achieve HiSCR100 at or after 16 weeks from the start of anti-IL-1β antibody treatment. In some embodiments, 30%-35% of the population of human subjects achieve HiSCR100 at or after 16 weeks from the start of anti-IL-1β antibody treatment. In some embodiments, 35%-40% of the population of human subjects achieve HiSCR100 at or after 16 weeks from the start of anti-IL-1β antibody treatment. In some embodiments, 40%-45% of the population of human subjects achieve HiSCR100 at or after 16 weeks from the start of anti-IL-1β antibody treatment. In some embodiments, 45%-50% of the population of human subjects achieve HiSCR100 at or after 16 weeks from the start of anti-IL-1β antibody treatment. In some embodiments, 50%-55% of the population of human subjects achieve HiSCR100 at or after 16 weeks from the start of anti-IL-1β antibody treatment. In some embodiments, 55%-60% of the population of human subjects achieve HiSCR100 at or after 16 weeks from the start of anti-IL-1β antibody treatment. In some embodiments, 60%-65% of the population of human subjects achieve HiSCR100 at or after 16 weeks from the start of anti-IL-1β antibody treatment. In some embodiments, 65%-70% of the population of human subjects achieve HiSCR100 at or after 16 weeks from the start of anti-IL-1β antibody treatment. In some embodiments, 70%-75% of the population of human subjects achieve HiSCR100 at or after 16 weeks from the start of anti-IL-1β antibody treatment. In some embodiments, 75%-80% of the population of human subjects achieve HiSCR100 at or after 16 weeks from the start of anti-IL-1β antibody treatment. In some embodiments, 80%-85% of the population of human subjects achieve HiSCR100 at or after 16 weeks from the start of anti-IL-1β antibody treatment. In some embodiments, 85%-90% of the population of human subjects achieve HiSCR100 at or after 16 weeks from the start of anti-IL-1β antibody treatment. In some embodiments, 90%-95% of the population of human subjects achieve HiSCR100 at or after 16 weeks from the start of anti-IL-1β antibody treatment. In some embodiments, 95%-100% of the population of human subjects achieve HiSCR100 at or after 16 weeks from the start of anti-IL-1β antibody treatment.

In some embodiments, administration of the anti-IL-1β antibody results in NRS30 in the population of human subjects. In some embodiments, at least 50% of the population of human subjects achieves NRS30. In some embodiments, each subject in the population of subjects has a Baseline NRS in PGA Skin Pain ≥3. In some embodiments, administration of the anti-IL-1β antibody results in at least a 30% reduction from baseline on a NRS in PGA Skin Pain. In some embodiments, administration of the anti-IL-1β antibody results in at least a 1-unit reduction from baseline on a NRS in PGA Skin Pain.

In some embodiments, administration of the anti-IL-1β antibody reduces hidradenitis suppurativa flares in the subject, wherein a flare is defined as ≥25% increase in AN count plus an increase of ≥2 in AN count. In some embodiments, the reduction is compared to baseline. In some embodiments, administration of the anti-IL-1β antibody increases the time between hidradenitis suppurativa flares in the subject. In some embodiments, the time between HS flares is 2 weeks, 4 weeks, 6 weeks, 8 weeks, 10 weeks, 12 weeks, 14 weeks, or 16 weeks. In some embodiments, the time between HS flares is an average time between HS flares, wherein the average is calculated based on the number of HS flares over the course of 3 months, 6 months, 9 months, 1 year, or 2 years. In some embodiments, the anti-IL-1β antibody is administered to a population of human subjects in need thereof, and wherein less than 10% of the population of human subjects experienced a hidradenitis suppurativa flare during the time period between administration of the loading dose and 16 weeks after administration of the loading dose. In some embodiments, less than 50%, 45%, 40%, 35%, 30%, 25%, 20%, 15%, or 5% of the population of human subjects experienced a hidradenitis suppurativa flare during the time period between administration of the loading dose and 16 weeks after administration of the loading dose. In some embodiments, less than 50%, 45%, 40%, 35%, 30%, 25%, 20%, 15%, or 5% of the population of human subjects experienced a hidradenitis suppurativa flare during the time period between administration of the loading dose and 16 weeks after the start of anti-IL-1β antibody treatment.

In some embodiments, the subject does not have (i.e., develop) anti-drug antibodies (ADAs) to the anti-IL-1β antibody. In some embodiments, the subject does not have ADAs at or after 2 weeks, 4 weeks, 6 weeks, 8 weeks, 10 weeks, 12 weeks, 14 weeks, 16 weeks, 18 weeks, or 20 weeks after the start of anti-IL-1β antibody treatment.

In some embodiments, the subject has failed treatment with an anti-TNF therapy. Examples of anti-TNF therapies include adalimumab. In some embodiments, the subject has failed treatment with a biologic therapy for HS. Examples of biologic therapies for HS include adalimumab, secukinumab, and bimekizumab. In some embodiments, the subject has failed treatment with adalimumab. In some embodiments, the subject has failed treatment with secukinumab. In some embodiments, the subject has failed treatment with bimekizumab. In some embodiments, the subject has failed treatment with an anti-TNF therapy for HS. In some embodiments, the subject has not failed treatment with an anti-TNF therapy. In some embodiments, the subject has not failed treatment with a biologic therapy for HS. In some embodiments, an anti-TNF therapy is contraindicated for the subject. In some embodiments, adalimumab is contraindicated for the subject. In some embodiments, a biologic therapy for HS is contraindicated for the subject. In some embodiments, secukinumab is contraindicated for the subject. In some embodiments, bimekizumab is contraindicated for the subject.

In some embodiments, the subject is receiving systemic antibiotics. In some embodiments, the systemic antibiotics are for the treatment of HS. In some embodiments, the subject is receiving systemic antibiotics at baseline and/or the subject is receiving systemic antibiotics during the study. In some embodiments, the antibiotic is doxycycline. In some embodiments, the antibiotic is minocycline. In some embodiments, the subject is receiving no more than 100 mg, twice daily, or is receiving the maximum dose of 100 mg, twice daily, of the systemic antibiotic. In some embodiments, the subject is receiving spironolactone. In some embodiments, the subject is receiving spironolactone at baseline and/or the subject is receiving spironolactone during the study. In some embodiments, the subject is on a stable regimen of spironolactone.

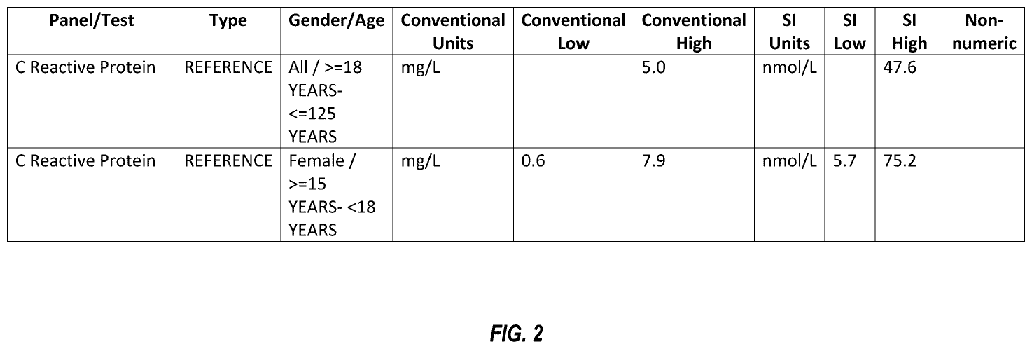

In some embodiments, the subject has a high level of high sensitivity C-reactive Protein (hs-CRP) at baseline. In some embodiments, the subject has a high level of IL-1β at baseline. The table of reference ranges in FIG. 2 shows results considered normal for high sensitivity C-reactive protein.

### Anti-IL-1β Antibodies

In some embodiments, the anti-IL-1β antibody useful for therapeutic purposes may comprise the CDR sequences and other sequences of the Mu007 antibody (SEQ ID NOS: 1-6), or of the W17, U43, W13, W18, or W20 antibodies comprising modified versions of the Mu007 antibody CDRs, which are provided in WO 2004/067568 and U.S. Pat. No. 7,541,033 B2 and U.S. Pat. No. 7,714,120 B2, each of which is incorporated herein by reference in their entireties, including the sequences in Tables and sequence listings.

In some embodiments, the anti-IL-1β antibody comprises: (i) a light chain complementarity determining region 1 (LCDR1) comprising the amino acid sequence of any one of SEQ ID NOs: 1, 12, 22, 29, 31, 33, 37, and 39; (ii) a light chain complementarity determining region 2 (LCDR2) comprising the amino acid sequence of SEQ ID NO: 2; (iii) a light chain complementarity determining region 3 (LCDR3) comprising the amino acid sequence of any one of SEQ ID NOs: 7, 13, 15, 17, 20, 25, 34, and 41; (iv) a heavy chain complementarity determining region 1 (HCDR1) comprising the amino acid sequence of one of SEQ ID NOs: 4, 10, and 71; (v) a heavy chain complementarity determining region 2 (HCDR2) comprising the amino acid sequence of any one of SEQ ID NOs: 5, 8, 16, 18, 21, 23, 28, and 72; and (vi) a heavy chain complementarity determining region 3 (HCDR3) comprising the amino acid sequence of any one of SEQ ID NOs: 6, 9, 11, 14, 19, 24, 26, 27, 30, 32, 35, 36, 38, 40, and 42.

In some embodiments, the anti-IL-1β antibody comprises a heavy chain comprising three CDR sequences comprising each of SEQ ID NOs: 10 (HCDR1), 21 (HCDR2), and 38 (HCDR3). In some embodiments, the anti-IL-1β antibody comprises a light chain comprising three CDR sequences comprising each of SEQ ID NOs: 39 (LCDR1), 2 (LCDR2), and 41 (LCDR3). In some embodiments, the anti-IL-1β antibody comprises a heavy chain comprising three CDR sequences comprising each of SEQ ID NOs: 10 (HCDR1), 21 (HCDR2), and 38 (HCDR3) and a light chain comprising three CDR sequences comprising each of SEQ ID NOs: 39 (LCDR1), 2 (LCDR2), and 41 (LCDR3). In some embodiments, the antibody comprises a heavy chain and a light chain, wherein the heavy chain comprises three CDR sequences comprising each of SEQ ID NOs: 10 (HCDR1), 21 (HCDR2), and 38 (HCDR3), and wherein the light chain comprises three CDR sequences comprising each of SEQ ID NOs: 39 (LCDR1), 2 (LCDR2), and 41 (LCDR3). In some embodiments, the anti-IL-1β antibody comprises a heavy chain variable region sequence (VH) comprising SEQ ID NO: 70 and a light chain variable region sequence (VL) comprising SEQ ID NO: 69. In some embodiments, the anti-IL-1β antibody comprises a heavy chain variable region sequence (VH) comprising a sequence that is at least 85%, 90%, 95%, 96%, 97%, 98%, or 99% identical to SEQ ID NO: 70 and a light chain variable region sequence (VL) comprising a sequence that is at least 85%, 90%, 95%, 96%, 97%, 98%, or 99% identical to SEQ ID NO: 69. In some embodiments, the anti-IL-1β antibody comprises a heavy chain variable region sequence (VH) comprising a sequence that is at least 85%, 90%, 95%, 96%, 97%, 98%, or 99% identical to SEQ ID NO: 70 and comprising three heavy chain CDR sequences comprising each of SEQ ID NOs: 10 (HCDR1), 21 (HCDR2), and 38 (HCDR3) and a light chain variable region sequence (VL) comprising a sequence that is at least 85%, 90%, 95%, 96%, 97%, 98%, or 99% identical to SEQ ID NO: 69 and comprising a light chain comprising three light chain CDR sequences comprising each of SEQ ID NOs: 39 (LCDR1), 2 (LCDR2), and 41 (LCDR3).

In some embodiments, the anti-IL-1β antibody comprises a heavy chain comprising three CDR sequences comprising each of SEQ ID NOs: 71 (HCDR1), 72 (HCDR2), and 38 (HCDR3). In some embodiments, the anti-IL-1β antibody comprises a light chain comprising three CDR sequences comprising each of SEQ ID NOs: 39 (LCDR1), 2 (LCDR2), and 41 (LCDR3). In some embodiments, the antibody comprises a heavy chain and a light chain, wherein the heavy chain comprises three CDR sequences comprising each of SEQ ID NOs: 71, 72, and 38, and wherein the light chain comprises three CDR sequences comprising each of SEQ ID NOs: 39 (LCDR1), 2 (LCDR2), and 41 (LCDR3). In some embodiments, the anti-IL-1β antibody comprises a heavy chain variable region sequence (VH) comprising SEQ ID NO: 70 and a light chain variable region sequence (VL) comprising SEQ ID NO: 69.

In some embodiments, the anti-IL-1β antibody comprises a heavy chain variable region sequence comprising SEQ ID NO: 70 or a sequence having at least 85%, at least 90%, at least 95%, at least 98%, or at least 99% identity to SEQ ID NO: 70. In some embodiments, the anti-IL-1β antibody comprises a light chain variable region sequence comprising SEQ ID NO: 69 or a sequence having at least 85%, at least 90%, at least 95%, at least 98%, or at least 99% identical to SEQ ID NO: 69. In some embodiments, the anti-IL-1β antibody comprises a heavy chain variable region sequence comprising SEQ ID NO: 70 or a sequence having at least 85%, at least 90%, at least 95%, at least 98%, or at least 99% identity to SEQ ID NO: 70, and a light chain variable region sequence comprising SEQ ID NO: 69 or a sequence having at least 85%, at least 90%, at least 95%, at least 98%, or at least 99% identical to SEQ ID NO: 69. In some embodiments, the anti-IL-1β antibody comprises a light chain variable region (VL) comprising an amino acid sequence that is at least 90%, 91%, 92%, 93%, 94%, 95%, 96%, 97%, 98%, or 99% identical to the amino acid sequence of SEQ ID NO: 69. In some embodiments, the anti-IL-1β antibody comprises a heavy chain variable region (VH) comprising an amino acid sequence that is at least 90%, 91%, 92%, 93%, 94%, 95%, 96%, 97%, 98%, or 99% identical to the amino acid sequence of SEQ ID NO: 70. In some embodiments, the anti-IL-1β antibody comprises a light chain variable region (VL) comprising an amino acid sequence that is at least 90%, 91%, 92%, 93%, 94%, 95%, 96%, 97%, 98%, or 99% identical to the amino acid sequence of SEQ ID NO: 69, and a heavy chain variable region (VH) comprising an amino acid sequence that is at least 90%, 91%, 92%, 93%, 94%, 95%, 96%, 97%, 98%, or 99% identical to the amino acid sequence of SEQ ID NO: 70. In some embodiments, the anti-IL-1β antibody comprises a light chain variable region (VL) comprising the amino acid sequence of SEQ ID NO: 69, and a heavy chain variable region (VH) comprising the amino acid sequence of SEQ ID NO: 70.

In some embodiments, the anti-IL-1β antibody comprises (i) a light chain complementarity determining region 1 (LCDR1) comprising the amino acid sequence of SEQ ID NO: 39; (ii) a light chain complementarity determining region 2 (LCDR2) comprising the amino acid sequence of SEQ ID NO: 2; (iii) a light chain complementarity determining region 3 (LCDR3) comprising the amino acid sequence of SEQ ID NO: 41; (iv) a heavy chain complementarity determining region 1 (HCDR1) comprising the amino acid sequence of one of SEQ ID NOs: 10 and 71; (v) a heavy chain complementarity determining region 2 (HCDR2) comprising the amino acid sequence of one of SEQ ID NOs: 21 and 72; (vi) a heavy chain complementarity determining region 3 (HCDR3) comprising the amino acid sequence of SEQ ID NO: 38; (vii) a variable light chain region (VL) comprising an amino acid sequence that has at least 80%, 85%, 90%, 95%, 97%, 98%, or 99% identity to SEQ ID NO: 69; and a variable heavy chain region (VH) comprising an amino acid sequence that has at least 80%, 85%, 90%, 95%, 97%, 98%, or 99% identity to SEQ ID NO: 70.

In some embodiments, the anti-IL-1β antibody comprises a heavy chain sequence comprising SEQ ID NO: 68 or a sequence having at least 85%, at least 90%, at least 91%, at least 92%, at least 93%, at least 94%, at least 95%, at least 96%, at least 97%, at least 98%, or at least 99% identity to SEQ ID NO: 68. In some embodiments, the anti-IL-1β antibody comprises a light chain sequence comprising SEQ ID NO: 48 or a sequence having at least 85%, at least 90%, at least 91%, at least 92%, at least 93%, at least 94%, at least 95%, at least 96%, at least 97%, at least 98%, or at least 99% identity to SEQ ID NO: 48. In some embodiments, the anti-IL-1β antibody comprises both a heavy chain comprising SEQ ID NO: 68 or a sequence that is at least 85%, at least 90%, at least 91%, at least 92%, at least 93%, at least 94%, at least 95%, at least 96%, at least 97%, at least 98%, or at least 99% identical to SEQ ID NO: 68 and a light chain comprising SEQ ID NO: 48 or a sequence that is at least 85%, at least 90%, at least 91%, at least 92%, at least 93%, at least 94%, at least 95%, at least 96%, at least 97%, at least 98%, or at least 99% identical to SEQ ID NO: 48. In some embodiments, the anti-IL-1β antibody comprises a heavy chain sequence comprising SEQ ID NO: 68 and a light chain sequence comprising SEQ ID NO: 48.

In some embodiments, the anti-IL-1β antibody comprises a heavy chain sequence comprising SEQ ID NO: 47 or a sequence having at least 85%, at least 90%, at least 91%, at least 92%, at least 93%, at least 94%, at least 95%, at least 96%, at least 97%, at least 98%, or at least 99% identity to SEQ ID NO: 47. In some embodiments, the anti-IL-1β antibody comprises both a heavy chain comprising SEQ ID NO: 47 or a sequence that is at least 85%, at least 90%, at least 91%, at least 92%, at least 93%, at least 94%, at least 95%, at least 96%, at least 97%, at least 98%, or at least 99% identical to SEQ ID NO: 47 and a light chain comprising SEQ ID NO: 48 or a sequence that is at least 85%, at least 90%, at least 91%, at least 92%, at least 93%, at least 94%, at least 95%, at least 96%, at least 97%, at least 98%, or at least 99% identical to SEQ ID NO: 48. In some embodiments, the anti-IL-1β antibody comprises a heavy chain sequence comprising SEQ ID NO: 47 and a light chain sequence comprising SEQ ID NO: 48.

In some embodiments, the anti-IL-1β antibody comprises an IgG1 heavy chain (HC). In some embodiments, the IgG1 heavy chain (HC) sequence comprises SEQ ID NOs: 45, 47, 49, 51, or 53 or a sequence having at least 85%, at least 90%, at least 91%, at least 92%, at least 93%, at least 94%, at least 95%, at least 96%, at least 97%, at least 98%, or at least 99% identity to SEQ ID NO: 45, 47, 49, 51.

In some embodiments, the IgG1 heavy chain (HC) is modified to include a YTE modification. The YTE modification refers to a human Fc domain containing the amino acid substitutions corresponding to M252Y/S254T/T256E (a YTE mutant). See, e.g., Lee et al., Nature Comm'c'ns (2019) 10:5031 (https://doi.org/10.1038/s41467-019-13108-2); Abdeldeim et al., Pharmaceutics (2023) 15:2402 (https://doi.org/10.3390/pharmaceutics15102402). In some embodiments, the IgG1 heavy chain (HC) sequence comprises a sequence having at least 85%, at least 90%, at least 91%, at least 92%, at least 93%, at least 94%, at least 95%, at least 96%, at least 97%, at least 98%, or at least 99% identity to SEQ ID NO: 45, 47, 49, 51, wherein the sequence comprises Met255Tyr, Ser257Thr, Thr259Glu.

In some embodiments, the anti-IL-1β antibody comprises a heavy chain sequence comprising SEQ ID NO: 73 or a sequence having at least 85%, at least 90%, at least 95%, at least 98%, or at least 99% identity to SEQ ID NO: 73. In some embodiments, the anti-IL-1β antibody comprises a light chain sequence comprising SEQ ID NO: 48 or a sequence having at least 85%, at least 90%, at least 91%, at least 92%, at least 93%, at least 94%, at least 95%, at least 96%, at least 97%, at least 98%, or at least 99% identity to SEQ ID NO: 48. In some embodiments, the anti-IL-1β antibody comprises both a heavy chain comprising SEQ ID NO: 73 or a sequence that is at least 85%, at least 90%, at least 91%, at least 92%, at least 93%, at least 94%, at least 95%, at least 96%, at least 97%, at least 98%, or at least 99% identical to SEQ ID NO: 73 and a light chain comprising SEQ ID NO: 48 or a sequence that is at least 85%, at least 90%, at least 91%, at least 92%, at least 93%, at least 94%, at least 95%, at least 96%, at least 97%, at least 98%, or at least 99% identical to SEQ ID NO: 48. In some embodiments, the anti-IL-1β antibody comprises a heavy chain sequence comprising SEQ ID NO: 73 and a light chain sequence comprising SEQ ID NO: 48.

In some embodiments, the anti-IL-1β antibody comprises a light chain (LC) comprising an amino acid sequence having at least 90%, 91%, 92%, 93%, 94%, 95%, 96%, 97%, 98%, or 99% sequence identity to any one of SEQ ID NOs: 46, 48, 50, 52, and 54, and a heavy chain (HC) comprising an amino acid sequence having at least 90%, 91%, 92%, 93%, 94%, 95%, 96%, 97%, 98%, or 99% sequence identity to any one of SEQ ID NOs: 45, 47, 49, 51, 53, 68, and 73.

In some embodiments, the anti-IL-1β antibody comprises a heavy chain comprising three CDR sequences comprising each of SEQ ID NOs: 10 (HCDR1), 21 (HCDR2), and 30 (HCDR3). In some embodiments, the anti-IL-1β antibody comprises a light chain comprising three CDR sequences comprising each of SEQ ID NOs: 29 (LCDR1), 2 (LCDR2), and 17 (LCDR3). In some embodiments, the antibody comprises a heavy chain and a light chain, wherein the heavy chain comprises three CDR sequences comprising each of SEQ ID NOs: 10 (HCDR1), 21 (HCDR2), and 30 (HCDR3), and wherein the light chain comprises three CDR sequences comprising each of SEQ ID NOs: 29 (LCDR1), 2 (LCDR2), and 17 (LCDR3).

In some embodiments, the anti-IL-1β antibody comprises a heavy chain comprising three CDR sequences comprising each of SEQ ID NOs: 10 (HCDR1), 21 (HCDR2), and 35 (HCDR3). In some embodiments, the anti-IL-1β antibody comprises a light chain comprising three CDR sequences comprising each of SEQ ID NOs: 37 (LCDR1), 2 (LCDR2), and 15 (LCDR3). In some embodiments, the antibody comprises a heavy chain and a light chain, wherein the heavy chain comprises three CDR sequences comprising each of SEQ ID NOs: 10 (HCDR1), 21 (HCDR2), and 35 (HCDR3), and wherein the light chain comprises three CDR sequences comprising each of SEQ ID NOs: 37 (LCDR1), 2 (LCDR2), and 15 (LCDR3).

In some embodiments, the anti-IL-1β antibody comprises a heavy chain comprising three CDR sequences comprising each of SEQ ID NOs: 10 (HCDR1), 16 (HCDR2), and 36 (HCDR3). In some embodiments, the anti-IL-1β antibody comprises a light chain comprising three CDR sequences comprising each of SEQ ID NOs: 39 (LCDR1), 2 (LCDR2), and 17 (LCDR3). In some embodiments, the antibody comprises a heavy chain and a light chain, wherein the heavy chain comprises three CDR sequences comprising each of SEQ ID NOs: 10 (HCDR1), 16 (HCDR2), and 36 (HCDR3), and the light chain comprises three CDR sequences comprising each of SEQ ID NOs: 39 (LCDR1), 2 (LCDR2), and 17 (LCDR3).

In some embodiments, the anti-IL-1β antibody comprises a heavy chain comprising three CDR sequences comprising each of SEQ ID NOs: 10 (HCDR1), 21 (HCDR2), and 35 (HCDR3). In some embodiments, the anti-IL-1β antibody comprises a light chain comprising three CDR sequences comprising each of SEQ ID NOs: 39 (LCDR1), 2 (LCDR2), and 17 (LCDR3). In some embodiments, the antibody comprises a heavy chain and a light chain, wherein the heavy chain comprises three CDR sequences comprising each of SEQ ID NOs: 10 (HCDR1), 21 (HCDR2), and 35 (HCDR3), and the light chain comprises three CDR sequences comprising each of SEQ ID NOs: 39 (LCDR1), 2 (LCDR2), and 17 (LCDR3).

In some embodiments, the anti-IL-1β antibody comprises the CDR sequences of the U2, U3, U4, U5, U6, U7, U8, U9, U10, U11, U12, U13, U14, U15, U16, U17, U18, U19, U20, U21, U22, U23, U24, U25, U26, U27, U28, U29, U30, U31, U32, U33, U34, U35, U36, U37, U38, U39, U40, U41, U44, V3, V4, V5, V6, V7, V8, W1, W2, W3, W4, W5, W6, W8, W9, W12, W14, W19, W21, or W22 antibodies described in U.S. Pat. No. 7,541,033 B2, which is incorporated by reference herein.

In some embodiments, the anti-IL-1β antibody comprises a heavy chain sequence comprising SEQ ID NO: 45 or a sequence having at least 85%, at least 90%, at least 91%, at least 92%, at least 93%, at least 94%, at least 95%, at least 96%, at least 97%, at least 98%, or at least 99% identity to SEQ ID NO: 45. In some embodiments, the anti-IL-1β antibody comprises a light chain sequence comprising SEQ ID NO: 46 or a sequence having at least 85%, at least 90%, at least 91%, at least 92%, at least 93%, at least 94%, at least 95%, at least 96%, at least 97%, at least 98%, or at least 99% identity to SEQ ID NO: 46. In some embodiments, the anti-IL-1β antibody comprises both a heavy chain comprising SEQ ID NO: 45 or a sequence that is at least 85%, at least 90%, at least 91%, at least 92%, at least 93%, at least 94%, at least 95%, at least 96%, at least 97%, at least 98%, or at least 99% identical to SEQ ID NO: 45 and a light chain comprising SEQ ID NO: 46 or a sequence that is at least 85%, at least 90%, at least 91%, at least 92%, at least 93%, at least 94%, at least 95%, at least 96%, at least 97%, at least 98%, or at least 99% identical to SEQ ID NO: 46.

In some embodiments, the anti-IL-1β antibody comprises a heavy chain sequence comprising SEQ ID NO: 49 or a sequence having at least 85%, at least 90%, at least 91%, at least 92%, at least 93%, at least 94%, at least 95%, at least 96%, at least 97%, at least 98%, or at least 99% identity to SEQ ID NO: 49. In some embodiments, the anti-IL-1β antibody comprises a light chain sequence comprising SEQ ID NO: 50 or a sequence having at least 85%, at least 90%, at least 91%, at least 92%, at least 93%, at least 94%, at least 95%, at least 96%, at least 97%, at least 98%, or at least 99% identity to SEQ ID NO: 50. In some embodiments, the anti-IL-1β antibody comprises both a heavy chain comprising SEQ ID NO: 49 or a sequence that is at least 85%, at least 90%, at least 91%, at least 92%, at least 93%, at least 94%, at least 95%, at least 96%, at least 97%, at least 98%, or at least 99% identical to SEQ ID NO: 49 and a light chain comprising SEQ ID NO: 50 or a sequence that is at least 85%, at least 90%, at least 91%, at least 92%, at least 93%, at least 94%, at least 95%, at least 96%, at least 97%, at least 98%, or at least 99% identical to SEQ ID NO: 50.

In some embodiments, the anti-IL-1β antibody comprises a heavy chain sequence comprising SEQ ID NO: 51 or a sequence having at least 85%, at least 90%, at least 91%, at least 92%, at least 93%, at least 94%, at least 95%, at least 96%, at least 97%, at least 98%, or at least 99% identity to SEQ ID NO: 51. In some embodiments, the anti-IL-1β antibody comprises a light chain sequence comprising SEQ ID NO: 52 or a sequence having at least 85%, at least 90%, at least 91%, at least 92%, at least 93%, at least 94%, at least 95%, at least 96%, at least 97%, at least 98%, or at least 99% identity to SEQ ID NO: 52. In some embodiments, the anti-IL-1β antibody comprises both a heavy chain comprising SEQ ID NO: 51 or a sequence that is at least 85%, at least 90%, at least 91%, at least 92%, at least 93%, at least 94%, at least 95%, at least 96%, at least 97%, at least 98%, or at least 99% identical to SEQ ID NO: 51 and a light chain comprising SEQ ID NO: 52 or a sequence that is at least 85%, at least 90%, at least 91%, at least 92%, at least 93%, at least 94%, at least 95%, at least 96%, at least 97%, at least 98%, or at least 99% identical to SEQ ID NO: 52.

In some embodiments, the anti-IL-1β antibody comprises a heavy chain sequence comprising SEQ ID NO: 53 or a sequence having at least 85%, at least 90%, at least 91%, at least 92%, at least 93%, at least 94%, at least 95%, at least 96%, at least 97%, at least 98%, or at least 99% identity to SEQ ID NO: 53. In some embodiments, the anti-IL-1β antibody comprises a light chain sequence comprising SEQ ID NO: 54 or a sequence having at least 85%, at least 90%, at least 91%, at least 92%, at least 93%, at least 94%, at least 95%, at least 96%, at least 97%, at least 98%, or at least 99% identity to SEQ ID NO: 54. In some embodiments, the anti-IL-1β antibody comprises both a heavy chain comprising SEQ ID NO: 53 or a sequence that is at least 85%, at least 90%, at least 91%, at least 92%, at least 93%, at least 94%, at least 95%, at least 96%, at least 97%, at least 98%, or at least 99% identical to SEQ ID NO: 53 and a light chain comprising SEQ ID NO: 54 or a sequence that is at least 85%, at least 90%, at least 91%, at least 92%, at least 93%, at least 94%, at least 95%, at least 96%, at least 97%, at least 98%, or at least 99% identical to SEQ ID NO: 54.

In some embodiments, the anti-IL-1β antibody is a monoclonal antibody. In some embodiments, the anti-IL-1β antibody is an antibody fragment. In some embodiments, the fragment is a Fab, Fab′, Fv, scFv or (Fab′)₂. In some embodiments, the anti-IL-1β antibody is a full-length antibody. In some embodiments, the anti-IL-1β antibody is an IgG antibody. In some embodiments, the anti-IL-1β antibody is an IgG1 antibody, an IgG2 antibody, an IgG3 antibody, or an IgG4 antibody. In some embodiments, the Fc region of the anti-IL-1β antibody comprises IgG1, IgG2, IgG3, or IgG4. In some embodiments, the anti-IL-1β antibody comprises a human IgG1 heavy chain constant region. In some embodiments, the anti-IL-1β antibody comprises a human IgG4 heavy chain constant region. In some embodiments, the anti-IL-1β antibody comprises a human IgG kappa light chain constant region. In some embodiments, the anti-IL-1β antibody is a humanized antibody.

In some embodiments, humanized anti-IL-1β antibodies are provided. Humanized antibodies are useful as therapeutic molecules because humanized antibodies reduce or eliminate the human immune response as compared to non-human antibodies, which can result in an immune response to an antibody therapeutic (such as the human anti-mouse antibody (HAMA) response), and decreased effectiveness of the therapeutic.

Typically, a non-human antibody is humanized to reduce immunogenicity to humans, while retaining the specificity and affinity of the parental non-human antibody. Generally, a humanized antibody comprises one or more variable domains in which CDRs, (or portions thereof) are derived from a non-human antibody, and FRs (or portions thereof) are derived from human antibody sequences. A humanized antibody, optionally, will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13: 1619-1633, and are further described, for example, in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of light or heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271:22611-22618).

In some embodiments, an antibody described herein comprises one or more human constant regions. In some embodiments, the human heavy chain constant region is of an isotype selected from IgA, IgG, and IgD. In some embodiments, an antibody described herein comprises a human IgG constant region. In some embodiments, when effector function is desirable, an anti-IL-1β antibody comprising a human IgG1 heavy chain constant region or a human IgG3 heavy chain constant region is selected. In some embodiments, when effector function is not desirable, an anti-IL-1β antibody comprising a human IgG4 or IgG2 heavy chain constant region is selected. In some embodiments, the human light chain constant region is of an isotype selected from κ and λ. In some embodiments, an antibody described herein comprises a human IgG4 heavy chain constant region. In some embodiments, an antibody described herein comprises a human IgG4 constant region and a human κ light chain.

As noted above, whether or not effector function is desirable may depend on the particular method of treatment intended for an antibody. Thus, in some embodiments, when effector function is desirable, an anti-IL-1β antibody comprising a human IgG1 heavy chain constant region or a human IgG3 heavy chain constant region is selected. In some embodiments, when effector function is not desirable, an anti-IL-1β antibody comprising a human IgG4 or IgG2 heavy chain constant region is selected.

In some embodiments, an antibody is provided in which deamidation is eliminated at position 55 of the heavy chain complementarity determining region 2 (HCDR2), and in some embodiments, said deamidation results in improved stability of the antibody.

In some embodiments, an antibody comprises a variant Fc region has at least one amino acid substitution compared to the Fc region of a wild-type IgG or a wild-type antibody. In some embodiments, the variant Fc region has two or more amino acid substitutions in the Fc region of the wild-type antibody. In some embodiments, the variant Fc region has three or more amino acid substitutions in the Fc region of the wild-type antibody. In some embodiments, the variant Fc region has at least one, two or three or more Fc region amino acid substitutions described herein. In some embodiments, the variant Fc region herein will possess at least about 80% homology with a native sequence Fc region and/or with an Fc region of a parent polypeptide. In some embodiments, the variant Fc region herein will possess at least about 90% homology with a native sequence Fc region and/or with an Fc region of a parent polypeptide. In some embodiments, the variant Fc region herein will possess at least about 95% homology with a native sequence Fc region and/or with an Fc region of a parent polypeptide. In some embodiments, a heavy chain constant region lacks the C-terminal lysine (K) residue, for example, because it is removed during antibody production. In some such embodiments, the heavy chain or heavy chain constant region may be referred to as “desK.” In some embodiments, the heavy chain constant region lacking the C-terminal lysine is an IgG, such as an IgG1, IgG2, IgG3, or IgG4. The heavy chain amino acid sequences provided herein do not include the terminal lysine. It is to be understood that any of the antibodies provided herein may be expressed with the terminal lysine, and/or may exist as a mixture of antibodies, some with the terminal lysine and some without the terminal lysine. Such mixtures typically arise when the expression sequence encodes the terminal lysine, but it is removed from some of the antibodies during production.

In some embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, for example, Wright et al. TIBTECH 15:26-32 (1997). The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (for example, complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, that is, between positions 294 and 300, due to minor sequence variations in antibodies. Such fucosylation variants may have improved ADCC function. See, for example, US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, for example, Yamane-Ohnuki et al. Biotech. Bioeng., 87: 614 (2004); Kanda, Y. et al., Biotechnol. Bioeng., 94(4):680-688 (2006); and WO2003/085107).

Antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, for example, in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, for example, in WO 1997/30087 (Patel et al.); WO 1998/58964 (Raju, S.); and WO 1999/22764 (Raju, S.).

Antibody variants are also provided with amino-terminal leader extensions. For example, one or more amino acid residues of the amino-terminal leader sequence are present at the amino-terminus of any one or more heavy or light chains of an antibody. An exemplary amino-terminal leader extension comprises or consists of three amino acid residues, VHS, present on one or both light chains of an antibody variant.

The in vivo or serum half-life of human FcRn high affinity binding polypeptides can be assayed, for example, in transgenic mice, in humans, or in non-human primates to which the polypeptides with a variant Fc region are administered. See also, for example, Petkova et al. International Immunology, 18(12):1759-1769 (2006).

### EXAMPLES

The following examples are provided to illustrate certain disclosed embodiments and are not to be construed as limiting the scope of this disclosure in any way. In the Examples discussed below, “Antibody A” refers to an anti-IL-1β antibody, wherein the anti-IL-1β antibody comprises the following six CDRs: a heavy chain CDR1 having an amino acid sequence of SEQ ID NO: 71; a heavy chain CDR2 having an amino acid sequence of SEQ ID NO: 72; a heavy chain CDR3 having an amino acid sequence of SEQ ID NO: 38; a light chain CDR1 having an amino acid sequence of SEQ ID NO: 39; a light chain CDR2 having an amino acid sequence of SEQ ID NO: 2; and a light chain CDR3 having an amino acid sequence of SEQ ID NO: 41. Antibody A has a variable heavy chain region (VH) having an amino acid sequence of SEQ ID NO: 70 and a variable light chain region (VL) having an amino acid sequence of SEQ ID NO: 69. Antibody A has a heavy chain having an amino acid sequence of SEQ ID NO: 68 (or SEQ ID NO: 73, which is SEQ ID NO: 68 without a leader sequence) and a light chain having an amino acid sequence of SEQ ID NO: 48.

### Example 1—A Phase II, Randomized, Double-Blind, Placebo-Controlled, Parallel-Group Study to Evaluate the Efficacy and Safety of Antibody A in Patients with Moderate to Severe Hidradenitis Suppurativa

### Example 1.1—Study Objectives And Endpoints

The primary objective of this study is to evaluate the efficacy of Antibody A versus placebo for the treatment of subjects with moderate to severe HS.

The secondary objectives of this study are: to assess the safety and tolerability of Antibody A in subjects with moderate to severe HS; to assess the efficacy of Antibody A versus placebo using additional outcome measures for the treatment of subjects with moderate to severe HS; and to evaluate the immunogenicity of Antibody A in subjects with moderate to severe HS.

The exploratory objectives of this study are: to evaluate the efficacy of Antibody A compared with placebo with respect to quality of life in subjects with moderate to severe HS; to characterize the pharmacokinetics (PK) of Antibody A in subjects with moderate to severe HS; and to evaluate changes in biomarkers of pharmacodynamic (PD) activity following treatment with Antibody A in subjects with moderate to severe HS.

The primary endpoint of this study is the proportion of subjects achieving HiSCR75 at Week 16 defined as: at least a 75% reduction in the total abscess and inflammatory nodule (AN) count, with no increase in abscess count and no increase in draining fistula count relative to Baseline.

The secondary endpoints of this study are: incidence of adverse events (AEs), and changes from Baseline in vital signs, physical examinations, and clinical laboratory tests; the proportion of subjects achieving HiSCR50 by visit defined as: at least a 50% reduction in the total AN count, with no increase in abscess count and no increase in draining fistula count relative to Baseline; the proportion of subjects achieving HiSCR90 by visit defined as: at least a 90% reduction in the total AN count, with no increase in abscess count and no increase in draining fistula count relative to Baseline; change from Baseline in International HS Severity Score System (IHS4); change from Baseline in AN count; change from Baseline in draining fistula count; percentage of subjects achieving NRS30; percentage of subjects with flares defined as ≥25% increase in AN count plus an increase of ≥2 in AN count compared to Baseline; and incidence of Antibody A ADA at specified timepoints.

The exploratory endpoints of this study are: change from Baseline in Hidradenitis Suppurativa Quality of Life Instrument (HiSQOL™); change from Baseline in Dermatology Life Quality Index (DLQI); change from Baseline in Patient Health Questionnaire (PHQ-9); serum concentrations of Antibody A at specified timepoints; and change from Baseline in serum/plasma biomarkers of PD activity, including levels of high sensitivity C-reactive Protein (hs-CRP) and IL-6.

### Example 1.2—Study Design

### Overall Study Design and Plan

This is a randomized, double-blind, placebo-controlled, parallel-group Phase II study with two Antibody A dose regimens to evaluate the efficacy and safety of Antibody A in adults with moderate to severe HS.

Following informed consent, subjects will undergo the procedures designated for the Screening Visit which will last from 7 up to 28 days. Those meeting eligibility criteria will receive access to and instruction on how to complete the electronic diary between 7 to 14 days prior to the planned Baseline Visit. Depending on the needs of the site and/or each individual subject, including the ability to utilize the electronic diary via a personal device versus a study supplied device, this may occur via an in-clinic, in person visit, or during a remote, telephone/virtual visit. Beginning 7 to 14 days prior to the Baseline Visit, subjects will complete the PGA Skin Pain assessment as well as an assessment of analgesic use (Yes/No) related to HS skin pain in the electronic diary. Subjects who complete less than 4 days of entries in the 7 days prior to Baseline are not to be randomized. At the Baseline Visit, upon re-confirmation of eligibility, subjects will be enrolled and randomized 1:1:1 to Antibody A 300 mg, Antibody A 150 mg, or placebo. The number of subjects who have not failed anti-TNF therapy (i.e., anti-TNF naïve, or anti-TNF exposed but not failure) will be limited to approximately 40%. The remainder of subjects will have failed anti-TNF therapy in the opinion of the Investigator. Note: anti-TNF failure is defined as a lack of response or loss of response and is not defined as an intolerance to anti-TNF therapy. In addition, the number of subjects who are receiving systemic antibiotics at Baseline (and continuing during the study) will be limited to approximately 20%. To allow for a more complete understanding of the PK profile of Antibody A, subjects will be assigned to a PK/ADA/PD sampling schedule at the time of randomization, as per the Schedule of Assessments (Table 1).

Randomization will be stratified by Hurley Stage (2 versus 3) and weight (90 kg versus >90 kg). Subjects will be dosed through Week 14. During the treatment period, subjects will complete the PGA Skin Pain assessment as well as record analgesic use (Yes/No) related to HS skin pain in the electronic diary daily (through Week 16) and return to the clinic for study drug administration and HS assessments every 2 weeks. At Week 16, subjects will complete the End of Treatment (EOT) procedures followed by a safety follow-up at Week 20 (6 weeks post last dose).

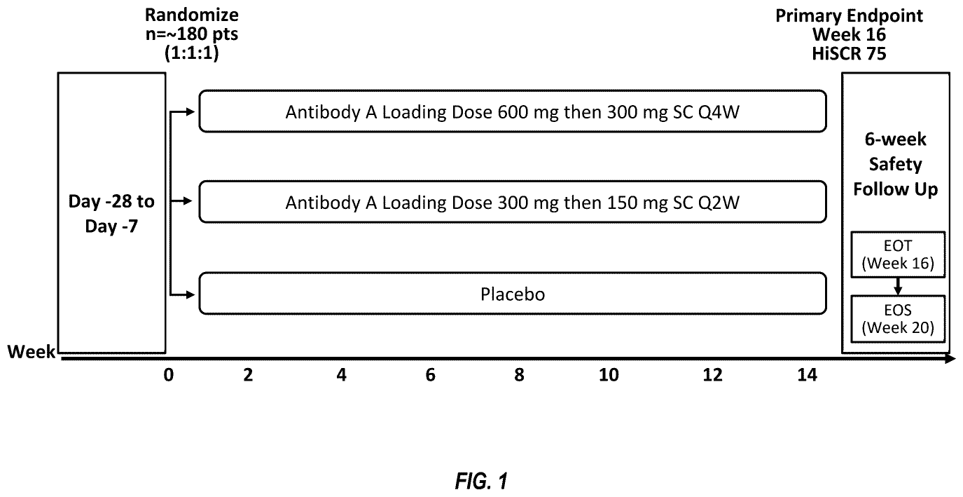

Safety monitoring will be performed continuously throughout the study in accordance with this protocol. All subjects will undergo efficacy, PK, ADA, and PD assessments. Subjects will also be monitored for AEs and will undergo safety laboratory tests as per the Schedule of Assessments (Table 1). A study schematic is provided in FIG. 1. Discussion of Study Design

This is the first clinical study where subjects with HS will be dosed with Antibody A; however, in previous clinical studies in subjects with rheumatoid arthritis (RA), type 2 diabetes mellitus (T2DM), and healthy subjects, Antibody A was shown to be well-tolerated.

The double-blind, placebo-controlled, parallel-group design of this study will enable the evaluation of the benefit-risk of two doses of Antibody A in an adequate and well-controlled setting. Due to concerns of keeping subjects with HS on placebo, subjects who are on a stable dose of select antibiotics (limited to approximately 20% of subjects) and/or a stable regimen of spironolactone at Baseline are permitted to remain on these medications, as described in Prior and Concomitant Therapies.

The primary endpoint (HiSCR) is a validated endpoint of treatment response in subjects with HS, which captures the more acute phase of HS activity that involves inflammatory changes, i.e., inflammatory nodules and abscess counts (Kimball A B, Sobell J M, Zouboulis C C, et al. J Eur Acad Dermatol Venereol 2016; 30(6):989-94). Additionally, HiSCR is an accepted endpoint for evaluation of HS and served as the basis for approval of secukinumab in adult subjects with moderate to severe HS (Cosentyx® United States Package Insert). Given that the assessment of severity of HS is complex and has remained controversial with numerous scoring systems available and none being universally accepted, this study includes several additional measures of disease severity as secondary endpoints, including both physician and subject-assessed measures. Additionally, given the psychological aspect of the disease, and impact on Quality of Life (QoL), several QoL tools have been included to measure the subject's wellbeing (Daoud M, Suppa M, Benhadou F, et al. Fron Med (Lausanne) 2023; 17:10:1145152).

### Dose Rationale

The loading dose (600 mg or 300 mg, SC) and maintenance dose (300 mg or 150 mg, SC) regimens were chosen to maximize efficacy (blockade of systemic IL-1β versus binding or neutralizing potency) while ensuring robust safety margins. A robust and durable response after Antibody A dosing will likely result from binding to and neutralization of IL-10, both systemically and in skin lesions. Modeling and simulation of systemic Antibody A concentrations using a previously published population PK model (Bihorel S, Fiedler-Kelly J, Ludwig E, et al. AAPS J 2014; 16(5):1009-17) reveals that both proposed dosing regimens should produce and maintain steady-state concentrations of Antibody A that greatly exceed the dissociation constant (KD) for binding to IL-1β (3 pM).

Steady-state trough concentrations, reached at approximately 100 days, are similar after both regimens, approximately 10 mg/mL or 68 nM, and are approximately 20,000-fold greater than the KD. At steady state, the 150 mg every 2 weeks and 300 mg every 4 weeks doses are proportional with respect to the area under the curve (AUC), but maximum and trough concentration (Cₘₐₓ and C_{trough}) values are nearly identical. This is most likely due to the faster absorption of the 150 mg dose (median time to maximum observed plasma concentration [Tₘₐₓ]=4 days) as compared to the 300 mg dose (median Tₘₐₓ=7 days).

Effective skin and lesion concentrations are also likely to be produced and maintained by both regimens. Shah D K, Betts A M. Mabs. 2013; 5(2):297-305 calculate the concentrations of antibodies, generally, in skin to be 16% of systemic plasma concentration. Assuming the same plasma:skin partitioning ratio, steady-state trough skin concentrations of 1.6 mg/mL or 11 nM would exceed the KD by approximately 4,000 fold.

The simulated steady-state concentrations of Antibody A align well with mean concentrations observed in 13 subjects in a Phase I study given a 150 mg SC dose of Antibody A; median Cₘₐₓ projected of 18.1 g/mL vs actual mean Cₘₐₓ of 17.5 g/mL. Accumulation at the lowest proposed doses, approximately 2-fold, is consistent with the expected accumulation given the apparent half-life of 17 days and the every-2-weeks maintenance dose administration.

Administering loading doses that quickly and safely saturate binding to circulating and tissue IL-1β may be important, given the unknown and unclear role immediately reducing IL-1β activity may play in quickly diminishing clinical symptoms in HS. Both loading doses achieve high systemic concentrations greatly exceeding the KD. Projected Cₘₐₓ and AUC values for the 600 mg and 300 mg loading doses are shown below (Table 3). For the loading doses, both Cₘₐₓ and AUC (normalized) are generally proportional to dose.

The dosing regimens to be studied in this clinical trial are within the limits of doses previously studied in both healthy volunteers and patients with inflammatory diseases based on AUC and Cₘₐₓ exposure. In the Phase I clinical trial, Antibody A was well-tolerated in patients with RA, T2DM, and healthy subjects.

### Start and End of Study Definitions

The study start is defined when the first informed consent form (ICF) is signed. The end of the study is reached when the last follow-up visit or contact, as planned according to the Schedule of Assessments (Table 1), for the last subject is performed.

### Selection of Study Population

Approximately 180 subjects aged ≥18 years with moderate to severe HS are planned to be enrolled to ensure approximately 162 subjects (54 subjects per treatment group) complete the study. The number of subjects who have not failed anti-TNF therapy (i.e., anti-TNF naïve, or anti-TNF exposed but not failure) will be limited to approximately 40%. The remainder of subjects will have failed anti-TNF therapy in the opinion of the Investigator (i.e., subject experienced lack of response or loss of response). In addition, the number of subjects who are receiving systemic antibiotics at Baseline (and continuing during the study) will be limited to approximately 20%.

### Inclusion Criteria

Subjects must fulfill the following requirements to be eligible for the study:
1. Subject is ≥18 years of age at the time of informed consent.
2. Subject can understand and provide written informed consent to participate in this study, agrees to comply with the requirements of the study, is able to read and understand study questionnaires, and has the ability to utilize an electronic diary.
3. Subject has signs and symptoms of HS for at least 6 months prior to Screening as determined by the Investigator (e.g., through review of medical history, interview of the subject).
4. Subject's HS is considered moderate or severe defined as: a total of at least 5 inflammatory lesions, i.e., an AN count of ≥5; AND HS inflammatory lesions in at least 2 distinct anatomic areas, at least one of which is Hurley Stage 2 or 3.
5. Subject is considered eligible according to the following tuberculosis (TB) screening criteria: has no history of latent or active TB, is not receiving concurrent treatment for TB, and does not show signs and/or symptoms of TB upon medical history review and/or physical examination at Screening. During Screening: Subject must have a negative QuantiFERON®-TB Gold test result, or in the case of subjects in countries where the QuantiFERON®-TB Gold test is not approved/registered or the tuberculin skin test is mandated by local health authorities, the subject must have a negative tuberculin skin test. Note: A subject whose first QuantiFERON®-TB Gold test result is indeterminate should have the test repeated. If the second QuantiFERON®-TB Gold test result is negative, the subject may be enrolled. If the second QuantiFERON®-TB Gold test result is positive or also indeterminate, the subject is excluded. All subjects must have a chest radiograph (posterior-anterior view) at Screening, or a chest radiograph/computed tomography (CT) taken within 3 months before Screening which has been read by a qualified radiologist or pulmonologist, with no evidence of current active or inactive TB. The report must be available for the Investigator's review.
6. Non-pregnant, non-lactating female subjects of childbearing potential who are heterosexually active and female sexual partners of childbearing potential of non-sterile male subjects must be using a highly effective method of contraception for 28 days prior to Baseline and agree to use a highly effective method of contraception during the treatment period and for 5 half-lives (14 weeks) following the last dose of study drug. A highly effective method of birth control is defined as one that results in a low failure rate (i.e., <1% per year) when used consistently and correctly, such as oral/injectable/intravaginal/implanted/transdermal contraceptives associated with inhibition of ovulation, intrauterine hormone-releasing system (IUS), intrauterine device (IUD), or true sexual abstinence.
   Note: True sexual abstinence is defined as refraining from heterosexual intercourse when this is in line with the preferred and usual lifestyle of the subject. Subjects who commit to abstinence must be counseled at the applicable study visits (Table 1) on the importance of maintaining abstinence and the potential risks of exposure of study drug to a developing embryo or fetus.
   Contraception is not required where at least 6 weeks have passed since sterilization, defined as females having undergone one of the following surgeries: hysterectomy, bilateral tubal ligation or occlusion, bilateral oophorectomy, or bilateral salpingectomy; and males who are vasectomized. Contraception is also not required where females are postmenopausal (defined as age ≥51 years and either 12 consecutive months of spontaneous amenorrhea OR 6 months of spontaneous amenorrhea with serum follicle stimulating hormone (FSH) levels >40 mIU/mL). Females of childbearing potential must have a negative (0-human chorionic gonadotropin) pregnancy test at Screening and a negative urine pregnancy test result at Baseline.
7. Subjects must agree not to donate eggs (ova, oocytes) or sperm for the purposes of assisted reproduction during participation in the study and for 5 half-lives (14 weeks) after the last study drug administration.

### Exclusion Criteria

The presence of any of the following criteria excludes a subject from the study:
1. Subject has a draining fistula count of ≥20.
2. Subject has another active skin inflammatory condition or infection (viral, bacterial, or fungal) which could interfere with the assessment of HS.
3. Subject has another active ongoing inflammatory disease (other than HS) that requires treatment with a prohibited medication.
4. Subject has a history of chronic or recurrent infectious disease, including but not limited to chronic renal infection, chronic lung infection, recurrent urinary tract infection, or open, draining or infected skin wounds or ulcers (not related to HS).
5. Subject has been hospitalized for an infection or received IV antibiotics for an infection during the 8 weeks prior to Screening.
6. Subject has known or suspected immunosuppression, including but not limited to a history of invasive opportunistic infection or solid organ transplant (excluding corneal transplantation performed >3 months prior to Screening).
7. Subject has a history of or laboratory evidence of human immunodeficiency virus (HIV) at Screening.
8. Subject has a history of symptomatic herpes zoster within the 30 days prior to Screening.
9. Subject has laboratory evidence of active or chronic Hepatitis B (HBV) as evidenced by: a positive Hepatitis B surface antigen test, OR a positive anti-Hepatitis B core antibody test, except in the circumstance of a negative HBV DNA PCR test, in which case the subject is permitted to enroll.
10. Subject has a history of or laboratory evidence of Hepatitis C virus (HCV) (HCV antibody positive) at Screening with the following exceptions: subjects with previously treated Hepatitis C with no recurrence for ≥1 year are permitted to enroll; subjects with negative HCV DNA PCR are permitted to enroll.
11. Subject has an active malignancy or lymphoproliferative disorder or a history thereof within the last 5 years, with the exception of resected non-melanoma (basal or squamous cell) skin cancer with no evidence of metastatic disease, or effectively managed cervical carcinoma in situ.
12. Subject has severe, progressive and/or uncontrolled renal, hepatic, hematologic, endocrine, pulmonary, cardiac (New York Heart Association Grade ≥3), neurologic or immunosuppressive disease.
13. Subject has had suicidal ideation or suicidal behavior within the previous 6 months or has severe depression as indicated by a PHQ-9 score of ≥20, or the subject answers anything other than “Not at all” to Question #9 on the PHQ-9 questionnaire at Screening or Baseline.
14. Subject has any other clinically significant acute or chronic medical condition(s) that, in the judgment of the Investigator, would preclude the use of a SC injection or the study drug that could compromise subject safety, limit the subject's ability to complete the study, and/or confound the results or compromise the objectives of the study.
15. Subject has had prior exposure to Antibody A or prior therapy with an anti-IL-1β targeting agent.
16. Subject has received a biologic immunosuppressive medication within 6 weeks or 5 half-lives, whichever is shorter, of Baseline.
17. Subject has received an immunosuppressive/immunomodulating medication within 28 days of Baseline.
18. Subject has received any live, attenuated vaccine within 3 months of Baseline.
19. Subject has received any other investigational agent within 5 half-lives (or 30 days for chemical agents, 8 weeks for biologic agents, whichever is longer) of Baseline.
20. Subject has used any topical therapy for HS within 14 days prior to the Baseline Visit. Note: over-the-counter antiseptics are permitted if the subject has been on a stable regimen for at least 14 days prior to Baseline, and there is no intent to change the regimen during the study period.
21. Subject has used any systemic therapies that may be effective for treating HS within 28 days prior to the Baseline Visit, with the following exceptions: doxycycline or minocycline (at a maximum dose of 100 mg, twice daily) is permitted provided the subject has been on a stable regimen for at least 28 days prior to Baseline, and there is no intent to change the regimen during the study period. Note: the number of subjects permitted to receive doxycycline or minocycline during the study period will be limited to approximately 20%; anti-androgens (e.g., spironolactone) are permitted, provided the subject has been on a stable dose for at least 12 weeks prior to Baseline and there is no intent to change the regimen during the study period. Combined oral contraceptives used for the purposes of birth control are permitted; metformin is permitted only if used for a non-HS related indication (e.g., diabetes), provided the subject has been on a stable dose for at least 28 days prior to Baseline and there is no intent to change the regimen during the study period.
22. Subject has received intralesional injections, surgical intervention (other than incision and drainage) or other physical modalities (e.g., light, laser, radiofrequency) for treatment of HS within 28 days prior to the Baseline Visit.
23. Subject has used opioid analgesics (including tramadol) within 14 days prior to Baseline, or it is anticipated that the subject will require opioid analgesics for any reason during the study period.
   Note: Subjects receiving non-opioid analgesics for the treatment of chronic HS-related pain must have consistent use for at least 14 days prior to the Baseline Visit. Pro re nata (PRN) use is not considered consistent use.
24. Subject has any of the following laboratory abnormalities:

$$\begin{matrix}a . & \\ Hemoglobin \leq 9. g / dL ( 90 g / L ) . & \end{matrix}$$

$$\begin{matrix}b . & \\ Neutrophils \leq 1 , 500 / {mm}^{3} ( 1.5 \times {10}^{9} / L ) . & \end{matrix}$$

$$\begin{matrix}c . & \\ Platelets \leq 100 , 000 / {mm}^{3} ( 100 \times {10}^{9} / L ) . & \end{matrix}$$

$$\begin{matrix}d . & \\ Serum creatinine > 1.5 mg / dL & \end{matrix}$$

$$\begin{matrix}e . & \\ Serum alanine aminotransferase ( ALT ) or aspartate \\ aminotransferase ( AST ) > 3 times the upper limit of normal ( ULN ) . & \end{matrix}$$

f. Total bilirubin >1.5 times the ULN, unless the subject has confirmed Gilbert's Disease.
g. Any other laboratory test results that pose an unacceptable risk for subject participation in the opinion of the Investigator.
Note: If one or more of the above laboratory parameters is out of range at Screening, a single retest of the laboratory value is permitted to qualify the subject without being considered a screen failure.
25. Female subject who is pregnant or is breastfeeding and will not abstain from breastfeeding during participation in the study and for 14 weeks after the last dose of study drug.
26. Subject has a history of clinically significant drug or alcohol abuse within the 12 months prior to Screening.
27. Subject has a known or suspected intolerance or hypersensitivity to the study drug, other biologics, or closely related compounds, or any ingredients of the study drug(s).
28. Subject for whom there is any concern on the part of the Investigator regarding the subject's legal or mental incapacitation, the subject's ability to understand the protocol or regarding the subject's safety, compliance, or suitability with respect to his/her participation in the study.

### Screen Failures

Subjects who fail inclusion and/or exclusion criteria may be rescreened once for the study with the prior approval of the Medical Monitor. For rescreening, all screening assessments must be performed as per protocol, with the exception of the chest X-ray for TB screening, provided it is not performed more than 12 weeks before rescreening.

### Premature Subject Withdrawal

All subjects will be advised that they are free to withdraw from participation in this study at any time, for any reason, and without prejudice. Every reasonable attempt should be made by the Investigator to keep subjects in the study; however, subjects must be withdrawn from the study if they withdraw consent to participate. Investigators must attempt to contact by telephone or other means subjects who fail to attend scheduled visits to exclude the possibility of an AE being the reason for the missed visit. Should this be the cause, the AE must be documented, reported, and followed as described in Adverse Event Collection.

A subject will be considered lost to follow-up if he or she repeatedly fails to return for scheduled visits and is unable to be contacted by the study site. A subject should not be considered lost to follow-up until all reasonable efforts made by site staff to contact the subject have been deemed futile. At a minimum, 3 attempts at contact (phone call, e-mail, text, and, if necessary, a certified letter to the subject's last known mailing address, or local equivalent method) should be made. Should these attempts fail, the subject will be considered as lost to follow-up.

Subjects can decline to continue receiving study drug at any time during the study. If this occurs, the Investigator is to discuss the completion of the End of Treatment (EOT) Visit, followed by completion of the End of Study (EOS) Visit which is to occur 6 weeks following the last dose of study drug. If the subject refuses the EOT and/or EOS Visit or procedures associated with this visit(s), data on concomitant medications and AE(s) will be collected if the subject agrees. Data on concomitant medications and AE(s) can be collected via a telephone call if the subject refuses an in-person visit.

Withdrawal of consent for a study means the subject does not wish to receive further protocol required treatment or procedures, and the subject does not wish to or is unable to continue further study participation. Subject data up to withdrawal of consent will be included in the analysis of the study, and where permitted, publicly available data may be included after withdrawal of consent.

The Sponsor reserves the right to request the withdrawal of a subject due to protocol deviations or other reasons. The Investigator also has the right to withdraw subjects from the study at any time for any reason.

If a subject is withdrawn before completing the study, the subject should be followed-up as instructed in the Schedule of Assessments (Table 1). The reason for withdrawal must be determined by the Investigator and recorded in the subject's medical record and in the electronic case report form (eCRF). If a subject is withdrawn for more than 1 reason, each reason should be documented in the source document and the most clinically relevant reason should be entered in the eCRF.

Reasons for discontinuation include but are not limited to: lack of efficacy (e.g., worsening HS requiring prohibited rescue/alternative therapy); AE; death; protocol-specified withdraw criteria met (see Protocol-Specified Withdrawal Criteria); protocol deviation; physician decision; sponsor request; withdrawal by subject; lost to follow-up; never dosed; and other (specify).

Subjects who are prematurely withdrawn from study drug should be followed up as instructed in the Schedule of Assessments (Table 1).

### Protocol-Specified Withdrawal Criteria

A subject must be withdrawn from study drug if they meet any of the following criteria: subject experiences any Grade 3 or higher hypersensitivity reaction, including serum sickness and/or cytokine release syndrome; subject becomes pregnant or plans to become pregnant during the study, or plans to donate eggs (ova, oocytes) or sperm during the study; subject develops a new malignancy; subject develops any clinically significant opportunistic infection; subject develops a serious infection during the study which would put the subject at risk for continuation of study drug; subject has a new diagnosis of TB; subject requires treatment with another immunosuppressive/immunomodulatory biologic, for any reason; subject requires treatment with another investigational agent; subject has any safety or tolerability issue, and the investigator believes that continuation of study drug is not in the subject's best interest; subject is unable to adhere to the study visit schedule or comply with protocol requirements such that the subject is at increased risk for continued participation in the study; subject has treatment unblinded by investigator; and/or subject has a laboratory or clinical finding suggestive of the potential for severe hepatic injury as defined below.

Subjects presenting with laboratory or clinical findings suggestive of the potential for severe hepatic injury, defined by the criteria below, should be discontinued from study drug (not temporarily interrupted), thoroughly investigated for other potential causes of liver injury, and followed closely through recovery. The following laboratory criteria require study drug discontinuation: alanine aminotransferase (ALT) or aspartate aminotransferase (AST)>8× the upper limit of normal (ULN); ALT or AST>5×ULN for more than 2 weeks; ALT or AST>3×ULN and (total bilirubin >2×ULN or international normalized ratio (INR)>1.5); and/or ALT or AST>3×ULN with the appearance of fatigue, nausea, vomiting, right upper quadrant pain or tenderness, fever, rash, and/or eosinophilia (>5%).

For subjects with significant elevations of aminotransferases in the opinion of the investigator that do not meet the individual stopping criteria (e.g., an isolated ALT or AST>3×ULN), study drug should be interrupted to permit repeat testing, inquiry into clinical symptoms, and investigation for other potential cause of liver injury. These subjects should undergo repeat laboratory testing within 48 to 72 hours (i.e., ALT, AST, alkaline phosphatase, total bilirubin), and close monitoring should be initiated.

TABLE 1
Schedule of Assessments

| | SCN | BL ^{a} | Double-Blind Treatment Period ^{b} | | | | | | | | | |
|---|---|---|---|---|---|---|---|---|---|---|---|---|
| | Visit | | | | | | | | | | | |
| | | | | | | | | | | | | 12/Fol- |
| | | | | | | | | | | | 11/ | low-up/ |
| | 1 | 2 | 3 | 4 | 5 | 6 | 7 | 8 | 9 | 10 | EOT ^{c} | EOS ^{c} |
| | Assessment Week | | | | | | | | | | | |
| | −4 | | | | | | | | | | | |
| | to −1 | 0 | 1 | 2 | 4 | 6 | 8 | 10 | 12 | 14 | 16 ^{c} | 20 ^{c} |
| | Assessment Day | | | | | | | | | | | |
| | −28 | | | | | | | | | | | |
| | to −7 | 1 | 8 | 15 | 29 | 43 | 57 | 71 | 85 | 99 | 113 | 141 |
| Visit Window (days) | NA | NA | ±1 | ±2 | ±2 | ±2 | ±2 | ±2 | ±2 | ±2 | ±2 | ±2 |
| Informed Consent ^{d} | X | | | | | | | | | | | |
| Inclusion/Exclusion | X | X | | | | | | | | | | |
| Review ^{e} | | | | | | | | | | | | |
| Medical and HS History/ | X | X | | | | | | | | | | |
| Demographics | | | | | | | | | | | | |
| Prior Medications ^{f} | X | X | | | | | | | | | | |
| Randomization | | X | | | | | | | | | | |
| Concomitant Medications | | X | X | X | X | X | X | X | X | X | X | X |
| Adverse Events ^{g} | X | X | X | X | X | X | X | X | X | X | X | X |
| Vital Signs, Weight, | X | X | X | X | X | X | X | X | X | X | X | X |
| and Height ^{h} | | | | | | | | | | | | |
| Physical Examination ⁱ | X | X | | | X | | X | | X | | X | X |
| Lesion Count ^{j} | X | X | | X | X | X | X | X | X | X | X | |
| Flare Assessment ^{k} | | | | X | X | X | X | X | X | X | X | |
| Hurley Stage^{j} | X | X | | X | X | X | X | X | X | X | X | |
| 12-Lead ECG ^{l} | X | | | | | | | | | | | |
| Chest Radiograph ^{m} | X | | | | | | | | | | | |
| PGA Skin Pain ⁿ | X | X | X | X | X | X | X | X | X | X | X | |
| Analgesic Use for | X | X | X | X | X | X | X | X | X | X | X | |
| HS Skin Pain ⁿ | | | | | | | | | | | | |
| Diary Instruction | X ^{o} | | | | | | | | | | | |
| Diary Review ^{p} | | X | X | X | X | X | X | X | X | X | X | |
| DLQI | | X | | | X | | X | | X | | X | |
| HiSQOL ™ | | X | | | X | | X | | X | | X | |
| IHS4 ^{k} | | X | | | X | | X | | X | | X | |
| PHQ-9 | X | X | | | X | | X | | X | | X | |
| PK, ADA and PD | | X | X | X | X | | X | X | X | | X | X |
| Sampling | | | | | | | | | | | | |
| Schedule 1 ^{q, r} | | | | | | | | | | | | |
| PK, ADA and PD | | X | X | | X | X | X | | X | X | X | X |
| Sampling | | | | | | | | | | | | |
| Schedule 2 ^{q, r} | | | | | | | | | | | | |
| QuantiFERON ® Test | X | | | | | | | | | | | |
| for TB ^{s} | | | | | | | | | | | | |
| Safety Laboratory | X | X | X | | X | | X | | X | | X | X |
| Tests (Hematology, | | | | | | | | | | | | |
| Chemistry) | | | | | | | | | | | | |
| Serum Pregnancy Test ^{t} | X | | | | | | | | | | X | X |
| Urine Pregnancy Test ^{t} | | X | | | X | | X | | X | | | |
| Contraception | X | X | | | X | | X | | X | | X | X |
| Counseling ^{u} | | | | | | | | | | | | |
| Study Drug | | X | | X | X | X | X | X | X | X | | |
| Administration ^{v} | | | | | | | | | | | | |
| Viral Screening ^{w} | X | | | | | | | | | | | |

ADA = anti-drug antibody; AE = adverse event; BL = Baseline; CT = computed tomography; DLQI = Dermatology Life Quality Index; ECG = electrocardiogram; eCRF = electronic case report form; ET = Early Termination; EOS = End of Study; EOT = End of Treatment; FSH = follicle stimulating hormone; HiSQOL ™ = Hidradenitis Suppurativa Quality of Life Instrument; HIV = human immunodeficiency virus; HS = hidradenitis suppurativa; hs-CRP = high-sensitivity C reactive protein; IHS4 = International hidradenitis suppurativa Severity Score System; IL = interleukin; NA = not applicable; PGA = Patient's Global Assessment; PD = pharmacodynamic(s); PHQ-9 = Patient Health Questionnaire; PK = pharmacokinetic(s); SAE = serious adverse event; SCN=Screening Visit; TB = tuberculosis.
^{a} At Baseline, all procedures should be performed prior to randomization and subsequent dosing.
^{b} On dosing days, all assessments and laboratory sample collection should be performed prior to dosing.
^{c} If a subject prematurely discontinues from study drug during the Double-blind Treatment Period, procedures for EOT are to be performed at the time of ET, followed by EOS procedures 6 weeks following the last dose of study drug. If a subject refuses the EOT and/or EOS Visit or selected procedures associated with this visit(s), data on concomitant medications and AEs will be collected if the subject agrees. Data on concomitant medications and AEs can be collected via a telephone call if the subject refuses an in-clinic visit.
^{d} Obtain informed consent prior to performing any study-specific procedures.
^{e} While the data that supports confirmation of meeting eligibility criteria may not all be captured in the eCRF, it must by supported by source documentation.
^{f} Prior therapies include all pharmacologic therapies received within 30 days prior to Screening, any immunosuppressive/immunomodulating medications (including biologics) ever received (regardless of indication) other than corticosteroids (which will only be recorded in the 30 days prior to Screening), all medications ever received for the treatment of HS, all excisional surgeries and unroofing procedures (punch debridement or surgical unroofing) ever performed for the treatment of HS, including anatomical site, and physical interventions for the treatment of HS within the 12 weeks prior to Screening including excisional surgery, unroofing procedures, incision and drainage, laser therapy (e.g., YAG), intralesional steroid injections, radiation therapy and photodynamic therapy. Prior treatment information must be captured in the eCRF.
^{g} After signing of informed consent but prior to first dose of study drug, AE collection will only include SAEs. Therefore, all non-serious events that occur after Screening but before the first dose of study drug should be recorded as medical history rather than an AE. After administration of study drug all AEs will be reported through end of study/early termination.
^{h} Vital signs to include systolic and diastolic blood pressure, temperature, heart rate, and body weight. In addition, height (without shoes) is to be recorded at Screening.
ⁱ Physical examination to include an evaluation of signs and symptoms of active TB and risk of exposure to TB.
^{j}Lesion count and Hurley stage will be entered in the electronic Clinical Outcomes Assessment (eCOA) system. For each anatomical region, Hurley stage will be determined as well as a counting of inflammatory nodules, abscesses, draining fistulas and non-draining fistulas. In addition, the presence or absence of non-inflammatory nodules, HS scars and pustules will be captured for each anatomical location. Every attempt should be made to have lesion count and Hurley stage assessed by the same individual for a given subject at each visit.
^{k} Will be calculated from lesion count data entered in the eCOA system and does not require any additional action from study staff.
^{l} Additional ECGs may be performed if clinically indicated.
^{m} All subjects must have a chest radiograph (posterior-anterior view) at Screening, or a chest radiograph/chest CT taken within 3 months before Screening which was read by a qualified radiologist or pulmonologist, with no evidence of current active or inactive TB, and report is available for Investigator's review.
ⁿ PGA Skin Pain and analgesic use (Yes/No) for HS skin pain is to be collected daily in the electronic diary beginning 7 to 14 days prior to Baseline through Week 16.
^{o} Following confirmation that the subject meets all eligibility criteria, the subject will receive access to and instruction on how to complete their electronic diary. This should occur 7 to 14 days prior to the planned Baseline Visit. Depending on the needs of the site and/or each individual subject, including ability to utilize the electronic diary via a personal device versus study supplied device, this may occur via an in-clinic, in person visit or during a remote, telephone/virtual visit.
^{p} Site staff will review diary entries on a regular basis for assessment of subject compliance. Subjects who are non-compliant will be counseled by the site. Subjects who are non-compliant and complete less than 4 days of entries in the 7 days prior to Baseline are not to be randomized.
^{q} To allow for a more complete understanding of the PK profile of Antibody A, subjects will be assigned a PK sampling schedule at the time of randomization. All subjects will have PK drawn at Baseline, Week 1, Week 4, Week 8, Week 12, EOT/ET, and EOS. In addition, 50% of subjects will have PK drawn at Week 2 and Week 10 (PK Schedule 1), and 50% will have PK drawn at Week 6 and Week 14 (PK Schedule 2).
^{r} ADA and PD sampling (including hs-CRP and IL-6) to be done for an individual subject on the same schedule as PK draws. In addition to the timepoints designated above, an immunogenicity (ADA) sample will be collected if an immunologically-related AE is reported (e.g., a skin reaction, lupus-like syndrome, unexplained thrombocytopenia).
^{s} In countries where the QuantiFERON ®-TB test is not registered/approved, or the tuberculin skin test is mandated by local health authorities, TB skin testing will be performed, and subjects will return in 48 to 72 hours to have the test result read.
^{t} Pregnancy testing is required for females of childbearing potential. A subject is not considered to be of childbearing potential if they are post-menopausal (defined as age > 51 years and either 12 consecutive months of spontaneous amenorrhea OR 6 months of spontaneous amenorrhea with serum FSH levels > 40 mIU/mL) or at least 6 weeks have passed since sterilization (defined as females having undergone one of the following surgeries: hysterectomy, bilateral tubal ligation or occlusion, bilateral oophorectomy, or bilateral salpingectomy; and males who are vasectomized). Contraception is not required in females not of childbearing potential.
^{u} Male or female subjects who have committed to true sexual abstinence as their chosen form of contraception will receive prespecified counseling at Screening, Baseline, and every 4 weeks thereafter, plus at EOS, on the importance of maintaining abstinence during the study and for 5 half-lives (14 weeks) post last dose of study drug due to the potential risks of exposure to study drug to a developing embryo or fetus. Counseling discussions must be documented.
^{v} Subjects will be required to remain in the clinic for at least 60 minutes after study drug administration at Baseline, and for 30 minutes after each subsequent study drug administration, for AE monitoring.
^{w} Viral Screening includes HIV, Hepatitis B virus, and Hepatitis C virus.

PK projections for Antibody A are included in Table 2 below.

TABLE 2
Projected Steady-state AUC, Cₘₐₓ,
C_{trough} and Tₘₐₓ for both Dosing Regimens

| Dosing | AUC_{τ, ss} | C_{max, ss} | C_{trough} | *T_{max, ss} |
|---|---|---|---|---|
| Regimen | (μg*day/mL) | (μg/mL) | (μg/mL) | (days) |
| 600 mg LD | 477.8 | 20.9 | 9.6 | 7.0 |
| 300 mg Q4W | (360.6, 614.9 | (15.8, 26.6) | (7.0, 13.4) | (4.0, 7.0) |
| 300 mg LD | 244.9 | 18.1 | 9.5 | 4.0 |
| 150 mg Q2W | (180.5, 324.8) | (13.0, 24.1) | (7.0, 15.6) | (4.0, 4.0) |

AUC = area under the plasma-concentration curve; AUC_{τ, ss} = area under the plasma-concentration curve over the dosing interval at steady-state; Cₘₐₓ = maximum observed concentration; C_{max, ss} = maximum observed concentration at steady-state; C_{trough} = concentration observed at trough; LD = Loading Dose; Tₘₐₓ = time to maximum observed concentration; T_{max, ss} = time to maximum observed concentration at steady-state; Q2W = every 2 weeks; Q4W = every 4 weeks.
*Calculated from the last steady-state dose.
Note:
Numbers in the table are median (inter-quartile range)

PK projections for Antibody A loading doses are included in Table 3 below.

TABLE 3
Loading Dose Exposure Parameters for Both Dosing Regimens

| | †AUC | Cₘₐₓ | Tₘₐₓ |
|---|---|---|---|
| Dosing Regimen | (μg*day/mL) | (μg/mL) | (days) |
| 600 mg LD | 529.9 | 25.04 | 7.0 |
| 300 mg Q4W | (402.7, 683.8) | (18.9, 32.9) | (7.0, 14.0) |
| 300 mg LD | 155.2 | 12.7 | 7.0 |
| 150 mg Q2W | (118.9, 210.4) | (9.8, 17.2) | (7.0, 15.0) |

AUC = area under the plasma-concentration curve; Cₘₐₓ = maximum observed concentration; LD = Loading Dose; Tₘₐₓ = time to maximum observed concentration; Q2W = every 2 weeks; Q4W = every 4 weeks.
Note:
Numbers in the table are median (inter-quartile range)
*Calculated from the last steady-
state dose.

### Example 1.3—Treatments

### Identification of Investigational Product, Dose and Mode of Administration

Antibody A is the investigational product that will be used in this study. Antibody A will be supplied in single-use glass vials containing ≥1 mL of a clear 150 mg/mL solution. Placebo will be supplied in volume-matched excipients in the same concentration as the matrix for Antibody A. Antibody A or placebo will be administered by SC injection in the thigh or abdomen. Antibody A will be dosed with a loading dose of 600 mg followed by 300 mg every 4 weeks or loading dose of 300 mg followed by 150 mg every 2 weeks.

### Treatments Administered

Study drug will be administered by qualified study personnel. Subjects will be required to remain in the clinic for AE monitoring for at least 60 minutes after study drug administration at Baseline, and for 30 minutes after each subsequent study drug administration.

Subjects will be dosed as described below. Due to the difference in dosing in the Antibody A treatment arms (i.e., dose frequency and dose level), subjects in the Antibody A arms will receive placebo injections at selected visits to maintain the blind.
1. Treatment Arm 1: Antibody A 300 mg SC every 4 weeks (Q4W)
   a. Day 1: Loading dose: Antibody A 600 mg (four 1-mL injections: 150 mg/mL each)
   b. Weeks 2, 6, 10, and 14: Placebo for blinding purposes (1 injection: 1 mL)
   c. Weeks 4, 8, and 12: Antibody A 300 mg (two 1-mL injections: 150 mg/mL each)
2. Treatment Arm 2: Antibody A 150 mg SC every 2 weeks (Q2W)
   a. Day 1: Loading dose: Antibody A 300 mg (two 1-mL injections: 150 mg/mL each) PLUS placebo (2 injections: 1 mL each)
   b. Weeks 2, 6, 10, and 14: Antibody A 150 mg (one 1-mL injection: 150 mg/mL)
   c. Weeks 4, 8, and 12: Antibody A 150 mg (one 1-mL injection: 150 mg/mL) PLUS placebo (1 injection: 1 mL)
3. Treatment Arm 3: Placebo
   a. Day 1: Loading dose: placebo (4 injections: 1 mL each)
   b. Weeks 2, 6, 10, and 14: placebo (1 injection: 1 mL)
   c. Weeks 4, 8, and 12: placebo (2 injections: 1 mL each)

### Blinding and Unblinding Treatment Assignment

This is a double-blind study. All subjects, Investigators, and study personnel involved in the conduct of the study will be blinded to treatment assignment.

If the treatment assignment is unblinded for an individual subject, the Investigator will be notified of that subject's treatment assignment without unblinding the treatment assignments for the remaining subjects in the study.

### Dose Adjustment Criteria

No dose adjustments are allowed.

### Prior and Concomitant Therapies

Pharmacologic therapies including but not limited to prescription medications, over-the-counter medications, supplements, antiseptics, cannabis products, and non-pharmacological treatments must be recorded on the appropriate eCRF page as described below.

### Prior Therapies

Prior therapies include any treatments received prior to the first dose of study drug. The following will be collected: all pharmacologic therapies as defined above received within 30 days prior to Screening; any immunosuppressive/immunomodulating medications (including biologics) ever received (regardless of indication), other than corticosteroids (corticosteroids would only be recorded in the 30 days prior to Screening); all medications ever received for the treatment of HS; all excisional surgeries and unroofing procedures (punch debridement or surgical unroofing) ever performed for the treatment of HS, including anatomical site; and physical interventions received for the treatment of HS within the 12 weeks prior to Screening, including excisional surgery, unroofing procedures, incision and drainage, laser therapy (e.g., YAG), intralesional steroid injections, radiation therapy, and photodynamic therapy.

Prior treatment information must be recorded on the appropriate eCRF page.

### Concomitant Therapies

Concomitant therapies refer to all pharmacological and non-pharmacological therapies taken or received on or after the first dose of study drug through the subject's last study visit. A therapy can be both prior and concomitant if taken both prior to and after first dose of study drug. Concomitant therapy information must be recorded on the appropriate eCRF page. Subjects will enter analgesic use (Yes/No) related to HS skin pain into the electronic dairy on a daily basis.

### Permitted and Prohibited Therapies

Medications or therapies considered necessary for the subject's welfare may be administered at the discretion of the Investigator.

Birth control is permitted, where considered to be highly effective (e.g., results in a low failure rate [i.e., <1% per year]) when used consistently and correctly, including oral/injectable/intravaginal/implanted/transdermal contraceptives associated with inhibition of ovulation, IUS or IUD, or sexual abstinence.

The medications/therapies listed below are prohibited during the study. The Medical Monitor should be contacted where further clarity is needed. The Medical Monitor must be notified in advance (or as soon as possible thereafter) of any instances in which prohibited therapies are administered.

Immunomodulatory or immunosuppressant systemic agents, including biologics are prohibited. Subjects who require administration of additional biologics must be discontinued from study drug. Subjects who require administration of other immunomodulatory/immunosuppressant agents must be discontinued from study drug unless otherwise agreed with the Medical Monitor.

Investigational compounds, other than Antibody A are prohibited. Subjects who require administration of other investigational compounds must be discontinued from study drug.

Systemic treatments that may be effective for treating HS are prohibited (e.g., acitretin, zinc supplements, apremilast), with the exceptions noted below. If there is any question if a medication is potentially effective for the treatment of HS, the Medical Monitor should be contacted. Use of systemic HS treatments other than those below must be discussed with the Medical Monitor to determine whether continued participation in the study is warranted.

Anti-androgens (e.g., spironolactone) are permitted if the subject has been on a stable regimen for at least 12 weeks prior to Baseline. The regimen should not be changed during the study.

Oral antibiotics for the treatment of HS are not permitted, with the exception of doxycycline or minocycline (maximum dose of 100 mg, twice daily), provided the subject has been on a stable regimen for 28 days prior to Baseline. The regimen should not be changed during the study. Note: the number of subjects permitted to receive doxycycline or minocycline during the study period will be limited to approximately 20%. Note: Antibiotics for treatment of AEs are permitted when medically necessary and do not require discontinuation from the study.

Metformin is permitted only if used for a non-HS related indication (e.g., diabetes), provided the subject has been on a stable dose for at least 28 days prior to Baseline. The regimen should not be changed during the study.

Combined oral contraceptives are permitted only for purposes of birth control. The regimen should not be changed during the study.

Topical agents for treatment of HS are prohibited, with the following exception: over the counter antiseptics are permitted if the subject has been on a stable regimen for 14 days prior to Baseline. The regimen should not be changed during the study.

Physical modalities (light, laser, radiofrequency) or surgical treatment of HS lesions are prohibited with the following exceptions: injection of up to 1.0 mL of intralesional triamcinolone acetonide at a maximum concentration of 10 mg/mL; and incision and drainage. Note: a maximum of 2 protocol allowed interventions (intralesional injection and/or incision and drainage) may be performed during the study. This includes 2 interventions on 2 different lesions during a single visit, or 1 intervention on any lesion on 2 separate visits (i.e., treatment of the same lesion on 2 separate visits or 2 different lesions on 2 separate visits).

Opioid analgesics are prohibited, except for tramadol up to a maximum of 400 mg in 24 hours for pain (HS or non-HS) not controlled by the following permitted analgesics: ibuprofen, not to exceed 800 mg every 6 hours and 3200 mg in 24 hours; and acetaminophen (paracetamol) not to exceed maximum dosage per local label. Note: Medication for the treatment of pain not related to HS should be limited as much as possible but may be used when needed and should be recorded in the eCRF. Marijuana may be used as permitted by local law (i.e., in regions where medical marijuana or recreational marijuana is legal), but it should not be used to treat HS pain and must be recorded in the eCRF. Note: subjects will enter analgesic use (Yes/No) related to HS skin pain into the electronic diary on a daily basis.

If discontinuation of study drug due to receipt of a prohibited therapy is necessary, the subject should have the EOT procedures performed at the time of ET as well as a final safety follow-up visit (EOS) 6 weeks after the last administration of study drug.

Subject's vaccination status should be reviewed in accordance with current immunization guidelines prior to study start. Live, attenuated vaccines should not be given within the 3 months prior to the first dose of study drug, during treatment with study drug, or for 3 months after last dose of study drug. It is recommended that any other vaccination be discussed with the Medical Monitor prior to administration.

Treatment after End of Study

Following discontinuation or completion of the study, subjects may be treated with non-study therapies at the discretion of the Investigator.

### Example 1.4—Study Procedures

### Study Duration

The sequence and maximum duration of the study periods will be as follows. The screening period is up to 28 days. The double-blind treatment period last dose is administered at Week 14. The End of Treatment Visit is 2 weeks after the last dose of study drug (i.e., Week 16). The End of Study Safety Follow-up is 6 weeks after the last dose of the study drug (i.e., Week 20). The maximum study duration for each subject is 24 weeks.

### Assessments

Subject-reported outcome assessments (i.e., subject-assessed questionnaires/scores) should be completed before any test, procedures, administration of study drug or other clinical assessments at each study visit to prevent influencing subject perceptions.

Vital signs and electrocardiograms (ECGs) should precede any invasive procedures, including blood sample collection.

All blood samples must be obtained prior to study drug administration. Times of all blood collections will be recorded in the source documentation.

### Medical History

A medical and surgical history will be taken at Screening and Baseline. All significant medical history findings, including any concomitant auto-inflammatory diseases, that have been present or active within the 5 years prior to the Screening Visit will be entered into the eCRF. Medical history findings that have not been present within the 5 years prior to Screening will be recorded if deemed by the Investigator as clinically relevant to the conduct of the study. Medical history should include reproductive status.

The subject's overall HS history will be captured at the Screening Visit. This includes but is not limited to the approximate date of first symptoms, approximate date of diagnosis, HS family history, smoking history (including current and/or previous use of tobacco, as well as the estimated number of pack-years based on the approximate consumption per year), and prior treatment including approximate start and stop dates. The extent of the subject's disease will be recorded in the eCRF.

Additionally, as described in Adverse Event Collection, AE collection after consent but prior to first dose of study drug will only include SAEs. Therefore, any non-serious events that occur after Screening but before first dose of study drug should be recorded as medical history rather than an AE. Clinically significant observations noted during Screening procedures (e.g., findings from laboratory testing, physical examination, vital signs, etc.) should also be entered as medical history.

### Hurley Stage

The Hurley Stages of HS are defined in Table 4. The Investigator will determine the Hurley Stage in each affected anatomical region at the time points specified in the Schedule of Assessments (Table 1). If more than 1 stage is present in a region, the worst Stage in each region should be documented. The subject is assigned Hurley Stage corresponding to the Hurley Stage of his or her worst involved anatomical region. The highest Hurley Stage assigned at Baseline will be entered into the eCRF at randomization for stratification purposes. Every attempt should be made to have Hurley Stage assessed by the same individual for a given subject at all visits.

TABLE 4
Hurley Stages of Hidradenitis Suppurativa

| Stage | Description |
|---|---|
| I | Abscess formation (single or multiple) without |
| | sinus tracts and cicatrization |
| II | Recurrent abscesses with tract formation and cicatrization; |
| | single or multiple widely separated lesions |
| III | Diffuse or near-diffuse involvement or multiple interconnected |
| | tracts and abscesses across the entire area. |

### Efficacy

### Hidradenitis Suppurativa Clinical Response (HiSCR)

The HiSCR will be calculated programmatically using Investigator (or designee) assessed lesion counts at the time points specified in the Schedule of Assessments (Table 1).

The HiSCR50/75/90 is defined as at least a 50/75/90% reduction in the total inflammatory lesion count (abscesses and inflammatory nodules) compared to Baseline, with no increase in abscesses or draining fistula compared to Baseline.

### Lesion Count

The location and extent of HS will be assessed by the Investigator (or designee) at the time points specified in the Schedule of Assessments (Table 1) by recording the anatomic location(s) of the disease, as well as the number of inflammatory nodules, abscesses, draining fistulae, and non-draining fistulae in each of the locations according to the following definitions. In addition, the presence or absence of non-inflammatory nodules, HS scars and pustules will be captured for each anatomical location according to the definitions below. Every attempt should be made to have lesion count assessed by the same individual for a given subject at all visits:

Inflammatory nodule: a raised, deep-seated, three-dimensional, round nodule, >10 mm in diameter that is tender and erythematous without evidence of fluctuance. A pyogenic granuloma is considered an inflammatory nodule, but papules and pustules are not inflammatory nodules.

Non-inflammatory nodule: a raised, deep-seated, three-dimensional, round nodule, >10 mm in diameter that is not tender or is minimally tender, without erythema or evidence of fluctuance.

Abscess: a circumscribed collection of purulent exudate frequently associated with swelling, erythema and other signs of inflammation, such as fluctuance, tenderness, and pain.

Draining Fistula: a pathologic passageway connecting to the skin surface from dermis or subcutaneous tissue that drains serous or purulent fluid, either spontaneously or by gentle palpation.

Non-draining fistula: a pathologic passageway connecting to the skin surface from dermis or subcutaneous tissue that does not drain serous or purulent fluid, either spontaneously or by gentle palpation.

HS Scars: all HS scars, excluding surgical scars

Pustules: circumscribed, elevated lesions filled with purulent fluid, less than 1 cm in size

### International Hidradenitis Suppurativa Severity Score System (IHS4)

The IHS4 will be calculated programmatically using Investigator (or designee) assessed lesion counts at the time points specified in the Schedule of Assessments (Table 1).

The IHS4 is a dynamic severity assessment of HS (Zouboulis C C, Tzellos T, Kyrgidis A, et al., on behalf of the EHSF Investigator Group. Br J Dermatol 2017; 177:1401-9). IHS4 score is arrived at by the number of nodules (multiplied by 1) plus the number of abscesses (multiplied by 2) plus the number of draining tunnels (multiplied by 4). A total score of 3 or less signifies mild disease, 4 to 10 signifies moderate disease, and 11 or higher signifies severe disease.

### Patient Global Assessment (PGA) of Skin Pain

The PGA of Skin Pain will be completed daily by the subject via the electronic diary as per the Schedule of Assessments (Table 1).

The skin pain NRS is a single-item measure to capture the subject's self-reported skin pain severity by rating the worst level of skin pain in the last 24 hours as a number on a numerical rating scale from 0 (no pain) to 10 (worst pain imaginable). Subjects will be instructed to complete their assessment via the electronic diary at approximately the same time each day, based on a recall period of the last 24 hours. Site staff will review diary entries on a regular basis for assessment of subject compliance. Subjects who are non-compliant will be counseled by the site.

### Hidradenitis Suppurativa Flares

The number of subjects with flares will be programmatically calculated based on Investigator (or designee) assessed lesion counts. A flare is defined as ≥25% increase in AN count plus an increase of ≥2 in AN count compared to Baseline.

### Hidradenitis Suppurativa Quality of Life Instrument (HiSQOL™)

The HiSQOL™ will be completed by the subject at the time points specified in the Schedule of Assessments (Table 1).

The HiSQOL™ is a 17-item HS specific questionnaire used to assess patient's QoL, with a recall period of seven days. Each item is structured as a Likert-type scale with five or seven response levels and, for all items, the score ranges from 0 (not at all) to 4 (extremely). The HiSQOL™ items address HS symptoms, psychosocial and emotional impacts, as well as activities and adaptations related to HS.

The total score for the HiSQOL™ is calculated from the sum of the 17 item scores, which has a total score range from 0 to 68. Additionally, the HiSQOL™ has three validated subscale scores that can be derived from the item scores: symptoms (with a score range of 0 to 16), psychosocial (with a score range of 0 to 20), and activities-adaptations (with a score range of 0 to 32), with a higher score indicating a higher level of symptomology in all subscales (J S Kirby et al. 2020; Br. J. Dermatol. 183(2):340-348).

### Dermatology Life Quality Index (DLQI)

The DLQI will be completed by the subject at the time points specified in the Schedule of Assessments (Table 1).

The DLQI is a dermatology-specific quality of life questionnaire (Finlay & Khan. Clin. 1994; Exp. Dermatol. 19(3):210-16 (1994) consisting of 10 questions concerning the subject's perception of the impact of skin disease on different aspects of their health-related quality of life over the last 7 days. Each question is scored on a 4-point Likert scale (3=very much; 2=a lot; 1=a little; 0=not at all/question not relevant/question unanswered). The DLQI is then calculated by adding the score of each question, resulting in a maximum of 30 and a minimum of 0. A score higher than 10 indicates that the subject's life is being severely affected by their skin disease.

### Patient Health Questionnaire (PHQ-9)

The PHQ-9 will be completed by the subject at the time points specified in the Schedule of Assessments (Table 1).

The PHQ-9 is a 9-item depressive symptom scale and diagnostic tool to assess the presence and severity of depressive symptoms and a possible depressive disorder. (L. Constantini et al. 2021; J. Affect. Disord. 279:473-83). The PHQ-9 is a component of the larger self-administered Patient Health Questionnaire but can be used as a stand-alone instrument.

Questions on the level of interest/pleasure in doing things (anhedonia), feeling down or depressed, sleep-related problems (sleeping too much/difficulty falling or staying asleep), low energy or fatigue, eating problems (poor appetite or eating too much), self-worth (feeling like a failure), ability to concentrate, psychomotor problems (speaking/moving slowly or fidgety/restless), and thoughts of suicide, are included. Responses range from “0” (Not at all) to “3” (nearly every day). A tenth question asks about the extent to which the previously mentioned symptoms make functioning in daily life difficult. The response to the tenth question is not factored into the final score; however, clinicians may use the response to help gauge the patient's level of impairment.

The total sum of the responses roughly indexes levels of depression. Scores range from 0 to 27. In general, a total of 10 or above is suggestive of the presence of depression.

Site personnel should review subject responses to the PHQ-9 questionnaire following completion at the designated visits and assess the need for further mental health assessment and/or intervention. The following actions are required for a subject who answers anything other than “Not at all” for question 9 or who has a score of ≥20: at Screening or Baseline—exclude the subject from the study; at visits following Baseline—contact the medical monitor as soon as possible regarding the subject's continuation in the study; and at any visit (from Screening through Week 16)—further assessment for depression and suicide risk is needed. The subject should be promptly referred to a qualified health practitioner for further evaluation.

### Safety

Safety and tolerability assessments will include the frequency and severity of AEs as well as the evaluation of changes in clinical laboratory values, vital signs, ECGs, and physical examination findings.

### Clinical Laboratory Safety Assessments

Samples for the following clinical laboratory tests will be collected at the time points specified in the Schedule of Assessments (Table 1).

Hematology tests include hemoglobin, hematocrit, red blood cell count, mean corpuscular hemoglobin, mean corpuscular hemoglobin concentration, mean corpuscular volume, platelet count (or estimate), and white blood cell count including differential.

Serum chemistry tests include sodium, potassium, chloride, bicarbonate, glucose, blood urea nitrogen, creatinine, creatinine phosphokinase, ALT, AST, gamma-glutamyl transferase, alkaline phosphatase, total bilirubin, calcium, magnesium, phosphorous, lactate dehydrogenase, uric acid, total protein, and albumin.

A serum/urine pregnancy test will be administered for females of childbearing potential.

Virology tests include HIV, Hepatitis B, and Hepatitis C.

Other tests include serum and plasma biomarkers, including hs-CRP and IL-6; serum Antibody A PK and anti-drug antibodies (ADA); and TB testing with QuantiFERON®. In countries where the QuantiFERON®-TB test is not registered/approved or the tuberculin skin test is mandated by local health authorities, TB skin testing will be performed, and subjects will return in 48 to 72 hours to have the test result read.

Fasting is not required for any study-specific laboratory sample.

Laboratory specimens will be collected and analyzed. Study personnel should ensure correct and non-expired laboratory kits are used for each visit. Due to the potential unblinding nature of hs-CRP, IL-6, PK, and ADA test results will not be provided to blinded study personnel during the study.

### Evaluation of Laboratory Values

The normal ranges of values for the central laboratory assessments in this study will be based on standard reference ranges.

If a laboratory value is out of the reference range, it is not necessarily clinically relevant. The Investigator must review laboratory results in a timely manner, evaluate the out-of-range values and document his/her review in the subject's source documentation.

### Clinical Examinations

### Vital Signs, Weight, and Height

Vital signs including systolic and diastolic blood pressure, temperature, heart rate, and body weight will be measured at the time points specified in the Schedule of Assessments (Table 1). Height (without shoes) will be measured only at Screening. Weight at Baseline will be entered into the eCRF at randomization for stratification purposes.

Additional blood pressure and heart rate measurements may be performed, as determined by the Investigator, to ensure appropriate monitoring of subject safety and accurate recording of vital sign measurements. Any changes from Baseline deemed clinically significant by the Investigator are to be recorded as AEs. If the clinically significant finding is a sign of a disease or syndrome, only the diagnosis should be reported as medical history or an AE in the eCRF.

### Electrocardiogram

A standard 12-lead ECG at Screening will be performed by qualified personnel after the subject has been supine for approximately 5 minutes as designated in the Schedule of Assessments (Table 1). Additional ECGs may be performed if clinically indicated. ECG recordings will be identified with the subject number, date, and time of the recording and a copy will be included with the subject's source documentation.

The Investigator must review ECGs. All ECG values which, in the Investigator's opinion, show clinically relevant or pathological changes during the study are to be discussed with the Medical Monitor, as necessary, and reported as AEs and followed, as described in Adverse Event Collection. If the clinically significant finding is a sign of a disease or syndrome, only the diagnosis should be reported as medical history or an AE in the eCRF.

### Physical Examination

A physical examination (including an evaluation of signs and symptoms of active TB and risk of exposure to TB) will be performed at the time points specified in the Schedule of Assessments (Table 1). Any clinically significant physical examination findings are to be reported as medical history or AEs and followed, as described in Adverse Event Collection. If the clinically significant finding is a sign of a disease or syndrome, only the diagnosis should be reported as medical history or an AE in the eCRF.

### Adverse Events

The Investigator is responsible for the detection and documentation of events meeting the criteria and definition of an AE or SAE described below in Adverse Event Collection. At each visit, the subject will be allowed time to spontaneously report any issues since the last visit or evaluation

Any clinically relevant observations made during each visit will also be considered AEs. AEs will be collected as described in Adverse Event Collection.

### Pharmacokinetics

Blood samples for PK analysis will be collected at the time points specified in the Schedule of Assessments (Table 1) and will be sent to a central laboratory/specialized laboratory for assessment of PK. Samples are to be collected prior to dosing.

Samples for PK will be processed according to the laboratory's standard operating procedures (SOPs) using a validated method.

### Immunogenicity

Blood samples for immunogenicity (ADA) analysis will be collected at time points specified in the Schedule of Assessments (Table 1) and will be sent to a central/specialized laboratory for analysis. Samples are to be collected prior to dosing. Additional samples will be collected if an immunologically-related AE is reported (e.g., a skin reaction, lupus-like syndrome, unexplained thrombocytopenia).

Samples for ADA will be processed according to the laboratory's SOPs using a validated, non-commercial assay testing for ADAs specifically against Antibody A.

### Pharmacodynamics

PD will be assessed by measuring levels of hs-CRP and IL-6 in samples obtained at the time points specified in the Schedule of Assessments (Table 1) and will be sent to a central/specialized laboratory for analysis. Samples are to be collected prior to dosing.

Samples for PD will be processed according to the laboratory's SOPs using a validated method(s).

### Example 1.5—Adverse Events

An adverse event (AE) is defined as any untoward medical occurrence in a patient or clinical investigation subject administered a pharmaceutical product that does not necessarily have a causal relationship with the product. An AE can therefore be any unfavorable and unintended sign (including a new, clinically important abnormal laboratory finding), symptom, or disease, temporally associated with the product, whether or not related to the product. AE's occurring after first dose of study drug (including during the SC injection) through EOS will be considered treatment emergent.

Additionally, an AE that occurred prior to dosing with study drug and increased in severity after start of dosing will also be considered treatment emergent.

After signing the informed consent, but prior to first dose of study drug, AE collection will only include SAEs. Therefore, non-serious events that occur after Screening but before the first dose of study drug should be recorded as medical history rather than an AE. Clinically significant observations noted during Screening procedures (e.g., findings from laboratory testing, physical examination, vital signs, etc.) should also be entered as medical history. After initiation of study drug, all events will be reported as AEs through the end of study/early termination. Where possible, a diagnosis rather than a list of symptoms should be recorded. If a diagnosis has not been made, then each symptom should be listed individually.

Events that are clearly consistent with the expected pattern of progression of HS should not be reported as AEs unless they meet the definition of an SAE. These data will be captured as efficacy assessments only. In most cases, the pattern of progression will be captured as part of the clinical assessments and subject reported outcome assessments included in the protocol. Every effort should be made to document progression through use of objective criteria. If there is any uncertainty as to whether an event is due to disease progression, it should be reported as an AE.

An AE that changes in severity over time should be recorded in the eCRF once at the highest severity with the following exception: an AE which begins as a non-serious event, which later meets the definition of an SAE, should be entered once for the non-serious portion of the AE, and then be re-recorded as a new event with the start date the day it became serious.

### Severity of Adverse Events

The medical assessment of clinical severity of an AE will be determined using the definitions outlined in Common Terminology Criteria for Adverse Events (CTCAE), Version 5.0 (Published Nov. 27, 2017 by the US Department of Health and Human Services, National Institutes of Health, National Cancer Institute). The CTCAE displays Grades 1 through 5 with unique clinical descriptions of severity for each AE. Grade 1 is defined as mild; asymptomatic or mild symptoms; or clinical or diagnostic observations only; or intervention not indicated. Grade 2 is defined as moderate; or minimal, local or non-invasive intervention indicated; or limiting age-appropriate instrumental activities of daily living (ADL). Grade 3 is defined as severe or medically significant but not immediately life-threatening; or hospitalization or prolongation of hospitalization indicated; or disabling; or limiting self-care ADL. Grade 4 is defined as life-threatening consequences; or urgent intervention indicated. Grade 5 is defined as Death related to AE.

The above grading guidelines should be used whenever possible. For AEs that cannot be graded by the use of CTCAE, the severity should be graded using mild (Grade 1), moderate (Grade 2), severe (Grade 3), life threatening (Grade 4), and fatal (Grade 5).

Please refer to the above-referenced CTCAE document for full description of CTCAE terms and instrumental and self-care ADLs. It is important to distinguish between severe AEs and SAEs. Severity is a classification of intensity whereas an SAE is an AE that meets serious criteria, as described in Serious Adverse Event Definition.

### Relationship Categorization

A physician Investigator must make the assessment of relationship to the investigational product for each AL. The Investigator should decide whether, in his or her medical judgment, there is a reasonable possibility that the event may have been caused by the investigational product. If there is no valid reason for suggesting a relationship, then the AL should be classified as “not related”.

Otherwise, the AE should be categorized per the guidelines below. The causality assessment must be documented in the source document and the eCRF (Table 5).

TABLE 5
Assessment of Relationship to Investigational Product

| Relationship | Description |
|---|---|
| Not Related | Exposure to investigational product has not occurred. |
| | OR |
| | The administration of investigational product and the occurrence of the |
| | AE are not reasonably related in time. |
| | OR |
| | The AE is considered likely to be related to an etiology other than the |
| | use of the investigational product, that is, there are no facts/evidence or |
| | arguments to suggest a causal relationship to the investigational product. |
| Possibly | The administration of the investigational product and the occurrence of |
| Related | the AE are reasonably related in time. |
| | AND |
| | The AE could not be explained equally well by factors or causes other |
| | than exposure to investigational product. |
| Probably | The administration of the investigational product and the occurrence of |
| Related | the AE are reasonably related in time. |
| | AND |
| | The AE is more likely explained by exposure to investigational product |
| | than by other factors or causes. |

### Outcome at the Time of Last Observation

The outcome at the time of last observation will be classified as: recovered/resolved; recovered/resolved with sequelae; recovering/resolving; not recovered/not resolved; fatal (see Fatal Outcome); or unknown.

### Serious Adverse Events

### Serious Adverse Event Definition

An SAE is any untoward medical occurrence, whether considered to be related to investigational product or not, that at any dose: results in death; is life threatening; requires inpatient hospitalization or prolongation of existing hospitalization; results in persistent or significant disability/incapacity; is a congenital anomaly; is an important medical event.

### Serious Adverse Event Onset and Resolution Dates

The onset date of the SAE is defined as the date the event meets serious criteria. The resolution date is the date an outcome is reached, or stabilization is achieved (i.e., the Investigator does not expect any further improvement or worsening of the event).

Any signs or symptoms experienced by the subject leading up to the onset date of the SAE or following the resolution date of the SAE must be recorded as an AE.

### Fatal Outcome

Fatal should only be designated as an outcome when the AE results in death. If more than 1 AE is possibly related to the subject's death, the outcome of death should be indicated for each such AE.

For other AEs, ongoing at the time of death that did not contribute to the subject's death, the outcome should be considered not resolved, without an end date recorded.

### Special Considerations

The following events will be considered as AEs of special interest during this study: hypersensitivity reactions, injection site reactions, leukopenia, neutropenia, serious infections, opportunistic infections, TB, and malignancies.

The Investigator will be required to carefully monitor subjects for any signs or symptoms of infection such as, but not limited to, increased body temperature, malaise, weight loss, sweats, cough, dyspnea, pulmonary infiltrates, or serious febrile systemic illness. Any newly diagnosed malignancy must be reported as an SAE.

### Example 1.6—Overdose and Medication Error

For this study, any dose of study drug greater than that prescribed in the protocol will be considered an overdose. Overdose events are only considered AEs or SAEs if there are associated clinical signs and symptoms or if the act of taking the excess study drug itself is an AE or SAE (e.g., suicide attempt).

### Example 1.7—Statistics

This section presents a summary of the planned statistical analyses.

Descriptive statistics for continuous data will include the number of subjects (n observed data), mean, standard deviation (SD), median, minimum value, and maximum value by treatment group. Summaries of change from Baseline variables will include only subjects who have both a Baseline value and corresponding value at the timepoint of interest.

Categorical summaries will show the frequency and percent of subjects in the levels associated with the variable summarized by treatment group. Percentages will be calculated based on the total number of subjects with non-missing data for the summary. For all analyses, a two-sided 0.05 Type I error rate will be used to determine statistical significance and all confidence intervals will be 95% confidence intervals unless otherwise stated. All summaries and statistical analyses described below will be completed using Version 9.4 or later of the SAS Statistical Analysis System (SAS Institute, Inc. Cary, NC).

Descriptive statistics, including figures where appropriate, will be employed in the analysis of all efficacy variables, PK, PD biomarkers, and immunogenicity (ADA) data.

A total sample size of 180 subjects randomized in a 1:1:1 ratio is planned for this study. The sample size was selected based on the primary effectiveness comparison of HiSCR75 at 16 weeks. For the power calculation, a HiSCR75 response rate at 16 weeks of 42.5% in active versus 18% in the control arms was assumed, based on the AbbVie lutikizumab HS study (NCT05139602; Kimball A B, et al. Presented at: American Academy of Dermatology; Mar. 8-12, 2024; San Diego, CA).

The hypothesis is as follows:

$${H}_{0} : {p}_{active} = {p}_{placebo} vs {H}_{1} : {p}_{active} \neq {p}_{placebo}$$

Where p_{active} is the HiSCR75 response rate in an active arm and p_{placebo} is the placebo response rate. This will be evaluated for each active arm with a two-sided 0.05 significance level and a Bonferroni adjustment applied to maintain an overall two-sided Type I error rate of 0.10 for the study.

For the sample size of 180 and an approximate 10% drop out rate, a sample size of 162 completers is expected (54 per group). Under the assumptions of 42.5% response in an active arm and 18% in the placebo arm, the power is 80% using a z-test (pooled) for the difference of two proportions with a two-sided alpha of 0.05. Power was evaluated using nQuery Version 9.3.0

Analysis sets of interest include but are not limited to: Intent-to-Treat Analysis Set (ITT): includes all subjects who are randomized in the study. Subjects will be categorized according to their randomized treatment group; Safety Analysis Set: includes all subjects who are randomized in the study and receive at least one dose of study drug. Subjects will be categorized according to their actual treatment group; PK Analysis Set: includes all subjects who receive active study drug and have evaluable PK data.

All primary and secondary efficacy analyses will be conducted using the ITT analysis set. All safety analyses including adverse events, laboratory evaluations, and vital sign evaluations as well as PD analyses and immunogenicity analyses will be conducted on the safety analysis set. The PK analysis set will be used for PK analyses.

For binary response endpoints, missing data will be imputed using non-response imputation (NRI). Additional sensitivity analyses may be included in the statistical analysis plan (e.g. multiple imputation).

No interim analysis is planned for this study.

The disposition of all subjects enrolled in this study will be summarized as the count and percentage by treatment group and completion/discontinuation status. Subjects who discontinue the study drug and/or study prematurely will be summarized as the count and percentage by treatment group and reason for discontinuation. The number of subjects in each analysis set will also be summarized by treatment group.

All subject data will be reviewed for the occurrence of protocol deviations. Prior to database lock, all protocol deviations will be reviewed and classified with respect to the potential to influence experimental outcomes. Protocol deviations will be summarized by treatment group.

The analysis of demographic and baseline data will be performed for the ITT Analysis Set. Demographic variables include age, gender, race, ethnicity, height, weight, and body mass index.

Demographics and other baseline characteristics will be summarized by treatment group using descriptive statistics.

Medical history terms will be coded using the Medical Dictionary for Regulatory Activities (MedDRA) and the number and percentage of subjects reporting any medical history will be summarized by system organ class (SOC), preferred term (PT) and treatment group.

Medications will be coded using the World Health Organization (WHO) Drug Dictionary. Prior and concomitant medications will be summarized for the safety analysis set by treatment group using descriptive statistics.

Exposure to study drug will be summarized for the safety analysis set by treatment group using descriptive statistics. Subjects will be summarized according to cumulative exposure.

The primary estimand, including intercurrent events and strategies to handle them will be included. Supplemental estimand(s) will also be considered.

The primary efficacy endpoint for this study of the proportion of subjects achieving Hidradenitis Suppurativa Clinical Response (HiSCR75) at 16 weeks is defined as subjects achieving at least a 75% reduction in the total abscess and inflammatory nodule (AN) count with no increase in abscess count and no increase in draining fistula count relative to Baseline. The primary efficacy endpoint will be analyzed for the ITT analysis set between treatment groups using a Mantel-Haenszel (MH) test of the difference in two proportions stratified by the randomization stratification factors. The estimated MH risk difference will be summarized along with the two-sided 95% CI using MH stratum weights and the Sato variance estimator. Each active arm will be compared to the placebo arm separately. The primary efficacy endpoint is expressed with the null hypothesis:

$${H}_{0} : {p}_{active} = {p}_{placebo} vs {H}_{1} : {p}_{active} \neq {p}_{placebo}$$

Where p_{active} is the proportion achieving HiSCR75 at 16 weeks in an Antibody A group and p_{placebo} is the proportion achieving HiSCR75 at 16 weeks in the placebo group. Results will be summarized by the count, proportion and 95% CI for the proportion for each treatment group and p-value.

The primary efficacy analysis will also be performed in selected subgroups and by the randomization strata to assess the consistency of the treatment effect.

Secondary efficacy endpoints that are proportions will be analyzed in the same manner as the primary efficacy endpoint. Any continuous endpoints will be analyzed using a mixed model for repeated measures (MMRM). No adjustments for multiplicity will be made for secondary endpoints and all p-values presented will be nominal p-values. Full details of all models will be included in the analysis plan. Secondary endpoints to be analyzed are: proportion of subjects achieving HiSCR50; proportion of subjects achieving HiSCR90; change from Baseline in IHS4; change from Baseline in AN count; change from Baseline in draining fistula count; proportion of subjects achieving NRS30; and proportion of subjects with flares (defined as ≥25% increase in AN count plus an increase of ≥2 in AN count compared to Baseline).

The following endpoints are considered exploratory in nature. They will be analyzed using MMRM. No adjustments for multiplicity will be made for exploratory endpoints and all p-values presented will be nominal p-values. Exploratory endpoints include: change from Baseline in HiSQOL™; change from Baseline in DLQI; and change from Baseline in PHQ-9.

Safety analyses will be conducted using data from the safety analysis set. Safety variables include TEAEs, clinical laboratory values, and vital signs. No formal inferential analyses will be conducted for safety variables, unless otherwise noted.

Adverse events will be coded using MedDRA. AE's occurring after first dose of study drug (including during the SC injection) through the EOS Visit will be considered treatment emergent. CTCAE version 5.0 will be used for grading of AE severity.

The number and percentage of subjects having at least one TEAE will be summarized by treatment group. The incidence of TEAEs will be summarized by treatment group, SOC, and PT using the MedDRA dictionary. Each subject will be counted only once per SOC and PT. Adverse event summaries will be provided for: overall TEAE summary; all TEAEs; TEAEs by relationship (not related, possibly related, probably related); TEAEs by severity grade; serious TEAEs; serious related TEAEs; TEAEs leading to study discontinuation; TEAEs of special interest; and fatal TEAEs.

Note that MedDRA version will be selected prior to the beginning of data collection and will be documented in the data management and statistical analysis plans.

Descriptive summaries for all reported values and change from Baseline values will be summarized by treatment group and visit, and all laboratory data will be listed. The following laboratory summaries will be provided: hematology and serum chemistry.

Additionally, laboratory values will be summarized by CTCAE toxicity grade (where possible), and well as by potential clinical significance.

Descriptive summaries for all reported values and change from Baseline values will be summarized by treatment group and visit and presented in listings. Additionally, vital signs meeting potential clinical significance will be summarized.

ECG results will be presented in listings.

PK parameters including Cmax AUC will be summarized using descriptive statistics by treatment group and visit and will include number of subjects (n), geometric mean, SD, coefficient of variation, and range (minimum, maximum). Median Tmax will be reported. Half-life may be calculated if data permit. Descriptive statistics for serum concentrations will include concentrations by individual, number of subjects with concentrations below the level of quantification (BLQ), mean, SD, coefficient of variation, median, minimum, and maximum. For descriptive summaries, serum concentrations reported as BLQ will be set to zero for the calculation of means.

All PD parameters will be summarized by dose level and visit.

The incidence of ADAs will be summarized by dose level and visit.

### Sequences

The following Table 6 provides the sequences referred to in this application.

TABLE 6
Table of Sequences

| SEQ | | |
|---|---|---|
| ID | | |
| NO | Description | Sequence |
| 1 | VL CDR1 | Lys Ala Ser Gln Asp Ile Asp Arg Tyr Leu Ser |
| 2 | VL CDR2 | Arg Val Lys Arg Leu Val Asp |
| 3 | VL CDR3 | Leu Gln Tyr Asp Glu Phe Pro Tyr Thr |
| 4 | VH CDR1 | Gly Tyr Thr Phe Ser Arg Tyr Trp Ile Glu |
| 5 | VH CDR2 | Glu Ile Leu Pro Gly Asn Gly Asn Ile Asn Tyr |
| | | Asn Glu Lys Phe Lys Gly |
| 6 | VH CDR3 | Ile Tyr Tyr Asp Tyr Asp Gln Gly Phe Thr Tyr |
| 7 | VL CDR3 | Val Gln Tyr Asp Glu Phe Asn Tyr Thr |
| 8 | VH CDR2 | Glu Ile Leu Pro Gly Thr Gly Thr Ile Asn Tyr |
| | | Asn Glu Lys Phe Lys Gly |
| 9 | VH CDR3 | Val Tyr Tyr Asp Tyr Asp Tyr Gly Phe Asp Tyr |
| 10 | VH CDR1 | Gly Tyr Thr Phe Asp Arg Tyr Trp Ile Glu |
| 11 | VH CDR3 | Val Tyr Tyr Asp Tyr Asp Tyr Gly Phe Asp Leu |
| 12 | VL CDR1 | Lys Ala Ser Gln Asp Ile Asp Arg Tyr Leu Thr |
| 13 | VL CDR3 | Ile Gln Tyr Asp Glu Phe Asn Tyr Thr |
| 14 | VH CDR3 | Val Tyr Tyr Asp Tyr Asp Tyr Gly Phe Asp Asn |
| 15 | VL CDR3 | Ile Gln Tyr Asp Glu Phe Pro Tyr Thr |
| 16 | VH CDR2 | Glu Ile Leu Pro Gly Ser Gly Thr Ile Asn Tyr |
| | | Asn Glu Lys Phe Lys Gly |
| 17 | VL CDR3 | Val Gln Tyr Asp Glu Phe Pro Tyr Thr |
| 18 | VH CDR2 | Glu Ile Leu Pro Gly Thr Gly Asp Ile Asn Tyr |
| | | Asn Glu Phe Lys Gly |
| 19 | VH CDR3 | Val Tyr Tyr Asp Tyr Asp Tyr Gly Phe |
| 20 | VL CDR3 | Ile Gln Tyr Asp Glu Phe Pro Tyr Leu |
| 21 | VH CDR2 | Glu Ile Leu Pro Gly Ser Gly Asp Ile Asn Tyr |
| | | Asn Glu Lys Phe Lys Gly |
| 22 | VL CDR1 | Lys Phe Ser Gln Asp Ile Asp Arg Tyr Leu Thr |
| 23 | VH CDR2 | Glu Ile Leu Pro Gly Ser Gly Asn Ile Asn Tyr |
| | | Asn Glu Lys Phe Lys Gly |
| 24 | VH CDR3 | Val Tyr Tyr Asp Tyr Asp Gln Gly Phe Asp Asn |
| 25 | VL CDR3 | Val Gln Tyr Asp Glu Phe Pro Tyr Leu |
| 26 | VH CDR3 | Val Tyr Tyr Asp Tyr Asp Tyr Gly Phe Thr Tyr |
| 27 | VH CDR3 | Val Tyr Tyr Asp Tyr Asp Tyr Gly Phe Thr Asn |
| 28 | VH CDR2 | Glu Ile Leu Pro Gly Thr Gly Asn Ile Asn Tyr |
| | | Asn Glu Lys Phe Lys Gly |
| 29 | VL CDR1 | Lys Ala Ser Gln Asp Ile Asp Arg Phe Leu Ser |
| 30 | VH CDR3 | Met Tyr Tyr Asp Tyr Asp Gln Gly Phe Ser Leu |
| 31 | VL CDR1 | Lys Ala Ser Gln Asp Ile Asp Arg Phe Leu Thr |
| 32 | VH CDR3 | Met Tyr Tyr Asp Tyr Asp Gln Gly Phe Thr Asn |
| 33 | VL CDR1 | Lys Ala Ser Gln Asp Ile Asp Arg Phe Leu Ser |
| 34 | VL CDR3 | Val Gln Tyr Asp Glu Phe Ala Tyr Thr |
| 35 | VH CDR3 | Met Tyr Tyr Asp Tyr Asp Gln Gly Phe Asp Tyr |
| 36 | VH CDR3 | Met Tyr Tyr Asp Tyr Asp Gln Gly Phe Asp Asn |
| 37 | VL CDR1 | Lys Phe Ser Gln Asp Ile Asp Arg Phe Leu Thr |
| 38 | VH CDR3 | Met Tyr Tyr Asp Tyr Asp Gln Gly Phe Asp Leu |
| 39 | VL CDR1 | Lys Phe Ser Gln Asp Ile Asp Arg Phe Leu Ser |
| 40 | VH CDR3 | Met Tyr Tyr Asp Tyr Asp Gln Gly Phe Thr Leu |
| 41 | VL CDR3 | Val Gln Tyr Asp Glu Phe Pro Tyr Gly |
| 42 | VH CDR3 | Met Tyr Tyr Asp Tyr Asp Gln Gly Phe Thr Tyr |
| 43 | VL of | atggacatga ggacccctgc tcagtttctt ggaatctttt |
| | reference | tcttctggtt tccaggtatc agatgtgaca tcaagatgac |
| | clone | ccagtctcca tcttccatgt atgcatctct aggagagaga |
| | (Antibody | gtcactatca cttgcaaggc gagtcaggac attgataggt |
| | Mu007) | atttaagttg gttccagcag aaaccaggga aatctcctaa |
| | (DNA | gaccctgatc tatcgtgtaa agagattggt agatggggtc |
| | sequence) | ccatcaaggt tcagtggcag cgcatctggg caagattatt |
| | | ctctcaccat cagcagcctg cagtatgaag atatgggaat |
| | | ttattattgt ctacagtatg atgagtttcc gtacacgttc |
| | | ggagggggga ccaagctgga aataaaa |
| 44 | VH of | atggaatgga cctgggtctt tctcttcctc ctgtcagtaa |
| | reference | ctgcaggtgt ccactcccag gttcagctgc agcagtctgg |
| | clone | agctgagctg atgaagcctg gggcctcagt gaagatatcc |
| | (Antibody | tgcaaggcta ctggctacac attcagtagg tattggatag |
| | Mu007) | agtggataaa gcagaggcct ggacatggcc ttgagtggat |
| | (DNA | tggagagatt ttacctggaa atggaaatat taactacaat |
| | sequence) | gagaagttca agggcaaggc cacaatctct gcagattctt |
| | | cctccgaaac agcctacatg caactcagca gcctgtcctc |
| | | tgaggactct gccgtctatt attgttcaac aatctactat |
| | | gattacgacc aggggtttac ttactggggc caagggactc |
| | | tggtcactgt ttctgca |
| 45 | Heavy | Gln Val Gln Leu Val Gln Ser Gly Ala Glu Val |
| | chain | Lys Lys Pro Gly Ser Ser Val Lys Val Ser Cys |
| | (Antibody | Lys Ala Ser Gly Tyr Thr Phe Asp Arg Tyr Trp |
| | W13) | Ile Glu Trp Val Arg Gln Ala Pro Gly Gln Gly |
| | | Leu Glu Trp Met Gly Glu Ile Leu Pro Gly Ser |
| | | Gly Asp Ile Asn Tyr Asn Glu Lys Phe Lys Gly |
| | | Arg Val Thr Ile Thr Ala Asp Lys Ser Thr Ser |
| | | Thr Ala Tyr Met Glu Leu Ser Ser Leu Arg Ser |
| | | Glu Asp Thr Ala Val Tyr Tyr Cys Ala Arg Met |
| | | Tyr Tyr Asp Tyr Asp Gln Gly Phe Asp Tyr Trp |
| | | Gly Gln Gly Thr Leu Val Thr Val Ser Ser Ala |
| | | Ser Thr Lys Gly Pro Ser Val Phe Pro Leu Ala |
| | | Pro Ser Ser Lys Ser Thr Ser Gly Gly Thr Ala |
| | | Ala Leu Gly Cys Leu Val Lys Asp Tyr Phe Pro |
| | | Glu Pro Val Thr Val Ser Trp Asn Ser Gly Ala |
| | | Leu Thr Ser Gly Val His Thr Phe Pro Ala Val |
| | | Leu Gln Ser Ser Gly Leu Tyr Ser Leu Ser Ser |
| | | Val Val Thr Val Pro Ser Ser Ser Leu Gly Thr |
| | | Gln Thr Tyr Ile Cys Asn Val Asn His Lys Pro |
| | | Ser Asn Thr Lys Val Asp Lys Lys Val Glu Pro |
| | | Lys Ser Cys Asp Lys Thr His Thr Cys Pro Pro |
| | | Cys Pro Ala Pro Glu Leu Leu Gly Gly Pro Ser |
| | | Val Phe Leu Phe Pro Pro Lys Pro Lys Asp Thr |
| | | Leu Met Ile Ser Arg Thr Pro Glu Val Thr Cys |
| | | Val Val Val Asp Val Ser His Glu Asp Pro Glu |
| | | Val Lys Phe Asn Trp Tyr Val Asp Gly Val Glu |
| | | Val His Asn Ala Lys Thr Lys Pro Arg Glu Glu |
| | | Gln Tyr Asn Ser Thr Tyr Arg Val Val Ser Val |
| | | Leu Thr Val Leu His Gln Asp Trp Leu Asn Gly |
| | | Lys Glu Tyr Lys Cys Lys Val Ser Asn Lys Ala |
| | | Leu Pro Ala Pro Ile Glu Lys Thr Ile Ser Lys |
| | | Ala Lys Gly Gln Pro Arg Glu Pro Gln Val Tyr |
| | | Thr Leu Pro Pro Ser Arg Asp Glu Leu Thr Lys |
| | | Asn Gln Val Ser Leu Thr Cys Leu Val Lys Gly |
| | | Phe Tyr Pro Ser Asp Ile Ala Val Glu Trp Glu |
| | | Ser Asn Gly Gln Pro Glu Asn Asn Tyr Lys Thr |
| | | Thr Pro Pro Val Leu Asp Ser Asp Gly Ser Phe |
| | | Phe Leu Tyr Ser Lys Leu Thr Val Asp Lys Ser |
| | | Arg Trp Gln Gln Gly Asn Val Phe Ser Cys Ser |
| | | Val Met His Glu Ala Leu His Asn His Tyr Thr |
| | | Gln Lys Ser Leu Ser Leu Ser Pro Gly Lys |
| 46 | Light chain | Asp Ile Gln Met Thr Gln Ser Pro Ser Ser Leu |
| | (Antibody | Ser Ala Ser Val Gly Asp Arg Val Thr Ile Thr |
| | W13) | Cys Lys Phe Ser Gln Asp Ile Asp Arg Phe Leu |
| | | Thr Trp Phe Gln Gln Lys Pro Gly Lys Ala Pro |
| | | Lys Ser Leu Ile Tyr Arg Val Lys Arg Leu Val |
| | | Asp Gly Val Pro Ser Arg Phe Ser Gly Ser Gly |
| | | Ser Gly Thr Asp Phe Thr Leu Thr Ile Ser Ser |
| | | Leu Gln Pro Glu Asp Phe Ala Thr Tyr Tyr Cys |
| | | Ile Gln Tyr Asp Glu Phe Pro Tyr Thr Phe Gly |
| | | Gly Gly Thr Lys Val Glu Ile Lys Arg Thr Val |
| | | Ala Ala Pro Ser Val Phe Ile Phe Pro Pro Ser |
| | | Asp Glu Gln Leu Lys Ser Gly Thr Ala Ser Val |
| | | Val Cys Leu Leu Asn Asn Phe Tyr Pro Arg Glu |
| | | Ala Lys Val Gln Trp Lys Val Asp Asn Ala Leu |
| | | Gln Ser Gly Asn Ser Gln Glu Ser Val Thr Glu |
| | | Gln Asp Ser Lys Asp Ser Thr Tyr Ser Leu Ser |
| | | Ser Thr Leu Thr Leu Ser Lys Ala Asp Tyr Glu |
| | | Lys His Lys Val Tyr Ala Cys Glu Val Thr His |
| | | Gln Gly Leu Ser Ser Pro Val Thr Lys Ser Phe |
| | | Asn Arg Gly Glu Cys |
| 47 | Heavy | Gln Val Gln Leu Val Gln Ser Gly Ala Glu Val |
| | chain | Lys Lys Pro Gly Ser Ser Val Lys Val Ser Cys |
| | (Antibody | Lys Ala Ser Gly Tyr Thr Phe Asp Arg Tyr Trp |
| | W17) | Ile Glu Trp Val Arg Gln Ala Pro Gly Gln Gly |
| | | Leu Glu Trp Met Gly Glu Ile Leu Pro Gly Ser |
| | | Gly Asp Ile Asn Tyr Asn Glu Lys Phe Lys Gly |
| | | Arg Val Thr Ile Thr Ala Asp Lys Ser Thr Ser |
| | | Thr Ala Tyr Met Glu Leu Ser Ser Leu Arg Ser |
| | | Glu Asp Thr Ala Val Tyr Tyr Cys Ala Arg Met |
| | | Tyr Tyr Asp Tyr Asp Gln Gly Phe Asp Leu Trp |
| | | Gly Gln Gly Thr Leu Val Thr Val Ser Ser Ala |
| | | Ser Thr Lys Gly Pro Ser Val Phe Pro Leu Ala |
| | | Pro Ser Ser Lys Ser Thr Ser Gly Gly Thr Ala |
| | | Ala Leu Gly Cys Leu Val Lys Asp Tyr Phe Pro |
| | | Glu Pro Val Thr Val Ser Trp Asn Ser Gly Ala |
| | | Leu Thr Ser Gly Val His Thr Phe Pro Ala Val |
| | | Leu Gln Ser Ser Gly Leu Tyr Ser Leu Ser Ser |
| | | Val Val Thr Val Pro Ser Ser Ser Leu Gly Thr |
| | | Gln Thr Tyr Ile Cys Asn Val Asn His Lys Pro |
| | | Ser Asn Thr Lys Val Asp Lys Lys Val Glu Pro |
| | | Lys Ser Cys Asp Lys Thr His Thr Cys Pro Pro |
| | | Cys Pro Ala Pro Glu Leu Leu Gly Gly Pro Ser |
| | | Val Phe Leu Phe Pro Pro Lys Pro Lys Asp Thr |
| | | Leu Met Ile Ser Arg Thr Pro Glu Val Thr Cys |
| | | Val Val Val Asp Val Ser His Glu Asp Pro Glu |
| | | Val Lys Phe Asn Trp Tyr Val Asp Gly Val Glu |
| | | Val His Asn Ala Lys Thr Lys Pro Arg Glu Glu |
| | | Gln Tyr Asn Ser Thr Tyr Arg Val Val Ser Val |
| | | Leu Thr Val Leu His Gln Asp Trp Leu Asn Gly |
| | | Lys Glu Tyr Lys Cys Lys Val Ser Asn Lys Ala |
| | | Leu Pro Ala Pro Ile Glu Lys Thr Ile Ser Lys |
| | | Ala Lys Gly Gln Pro Arg Glu Pro Gln Val Tyr |
| | | Thr Leu Pro Pro Ser Arg Asp Glu Leu Thr Lys |
| | | Asn Gln Val Ser Leu Thr Cys Leu Val Lys Gly |
| | | Phe Tyr Pro Ser Asp Ile Ala Val Glu Trp Glu |
| | | Ser Asn Gly Gln Pro Glu Asn Asn Tyr Lys Thr |
| | | Thr Pro Pro Val Leu Asp Ser Asp Gly Ser Phe |
| | | Phe Leu Tyr Ser Lys Leu Thr Val Asp Lys Ser |
| | | Arg Trp Gln Gln Gly Asn Val Phe Ser Cys Ser |
| | | Val Met His Glu Ala Leu His Asn His Tyr Thr |
| | | Gln Lys Ser Leu Ser Leu Ser Pro Gly Lys |
| 48 | Light chain | Asp Ile Gln Met Thr Gln Ser Pro Ser Ser Leu |
| | (Antibody | Ser Ala Ser Val Gly Asp Arg Val Thr Ile Thr |
| | W17) | Cys Lys Phe Ser Gln Asp Ile Asp Arg Phe Leu |
| | | Ser Trp Phe Gln Gln Lys Pro Gly Lys Ala Pro |
| | | Lys Ser Leu Ile Tyr Arg Val Lys Arg Leu Val |
| | | Asp Gly Val Pro Ser Arg Phe Ser Gly Ser Gly |
| | | Ser Gly Thr Asp Phe Thr Leu Thr Ile Ser Ser |
| | | Leu Gln Pro Glu Asp Phe Ala Thr Tyr Tyr Cys |
| | | Val Gln Tyr Asp Glu Phe Pro Tyr Gly Phe Gly |
| | | Gly Gly Thr Lys Val Glu Ile Lys Arg Thr Val |
| | | Ala Ala Pro Ser Val Phe Ile Phe Pro Pro Ser |
| | | Asp Glu Gln Leu Lys Ser Gly Thr Ala Ser Val |
| | | Val Cys Leu Leu Asn Asn Phe Tyr Pro Arg Glu |
| | | Ala Lys Val Gln Trp Lys Val Asp Asn Ala Leu |
| | | Gln Ser Gly Asn Ser Gln Glu Ser Val Thr Glu |
| | | Gln Asp Ser Lys Asp Ser Thr Tyr Ser Leu Ser |
| | | Ser Thr Leu Thr Leu Ser Lys Ala Asp Tyr Glu |
| | | Lys His Lys Val Tyr Ala Cys Glu Val Thr His |
| | | Gln Gly Leu Ser Ser Pro Val Thr Lys Ser Phe |
| | | Asn Arg Gly Glu Cys |
| 49 | Heavy | Gln Val Gln Leu Val Gln Ser Gly Ala Glu Val |
| | chain | Lys Lys Pro Gly Ser Ser Val Lys Val Ser Cys |
| | (Antibody | Lys Ala Ser Gly Tyr Thr Phe Asp Arg Tyr Trp |
| | W18) | Ile Glu Trp Val Arg Gln Ala Pro Gly Gln Gly |
| | | Leu Glu Trp Met Gly Glu Ile Leu Pro Gly Ser |
| | | Gly Thr Ile Asn Tyr Asn Glu Lys Phe Lys Gly |
| | | Arg Val Thr Ile Thr Ala Asp Lys Ser Thr Ser |
| | | Thr Ala Tyr Met Glu Leu Ser Ser Leu Arg Ser |
| | | Glu Asp Thr Ala Val Tyr Tyr Cys Ala Arg Met |
| | | Tyr Tyr Asp Tyr Asp Gln Gly Phe Asp Asn Trp |
| | | Gly Gln Gly Thr Leu Val Thr Val Ser Ser Ala |
| | | Ser Thr Lys Gly Pro Ser Val Phe Pro Leu Ala |
| | | Pro Ser Ser Lys Ser Thr Ser Gly Gly Thr Ala |
| | | Ala Leu Gly Cys Leu Val Lys Asp Tyr Phe Pro |
| | | Glu Pro Val Thr Val Ser Trp Asn Ser Gly Ala |
| | | Leu Thr Ser Gly Val His Thr Phe Pro Ala Val |
| | | Leu Gln Ser Ser Gly Leu Tyr Ser Leu Ser Ser |
| | | Val Val Thr Val Pro Ser Ser Ser Leu Gly Thr |
| | | Gln Thr Tyr Ile Cys Asn Val Asn His Lys Pro |
| | | Ser Asn Thr Lys Val Asp Lys Lys Val Glu Pro |
| | | Lys Ser Cys Asp Lys Thr His Thr Cys Pro Pro |
| | | Cys Pro Ala Pro Glu Leu Leu Gly Gly Pro Ser |
| | | Val Phe Leu Phe Pro Pro Lys Pro Lys Asp Thr |
| | | Leu Met Ile Ser Arg Thr Pro Glu Val Thr Cys |
| | | Val Val Val Asp Val Ser His Glu Asp Pro Glu |
| | | Val Lys Phe Asn Trp Tyr Val Asp Gly Val Glu |
| | | Val His Asn Ala Lys Thr Lys Pro Arg Glu Glu |
| | | Gln Tyr Asn Ser Thr Tyr Arg Val Val Ser Val |
| | | Leu Thr Val Leu His Gln Asp Trp Leu Asn Gly |
| | | Lys Glu Tyr Lys Cys Lys Val Ser Asn Lys Ala |
| | | Leu Pro Ala Pro Ile Glu Lys Thr Ile Ser Lys |
| | | Ala Lys Gly Gln Pro Arg Glu Pro Gln Val Tyr |
| | | Thr Leu Pro Pro Ser Arg Asp Glu Leu Thr Lys |
| | | Asn Gln Val Ser Leu Thr Cys Leu Val Lys Gly |
| | | Phe Tyr Pro Ser Asp Ile Ala Val Glu Trp Glu |
| | | Ser Asn Gly Gln Pro Glu Asn Asn Tyr Lys Thr |
| | | Thr Pro Pro Val Leu Asp Ser Asp Gly Ser Phe |
| | | Phe Leu Tyr Ser Lys Leu Thr Val Asp Lys Ser |
| | | Arg Trp Gln Gln Gly Asn Val Phe Ser Cys Ser |
| | | Val Met His Glu Ala Leu His Asn His Tyr Thr |
| | | Gln Lys Ser Leu Ser Leu Ser Pro Gly Lys |
| 50 | Light chain | Asp Ile Gln Met Thr Gln Ser Pro Ser Ser Leu |
| | (Antibody | Ser Ala Ser Val Gly Asp Arg Val Thr Ile Thr |
| | W18) | Cys Lys Phe Ser Gln Asp Ile Asp Arg Phe Leu |
| | | Ser Trp Phe Gln Gln Lys Pro Gly Lys Ala Pro |
| | | Lys Ser Leu Ile Tyr Arg Val Lys Arg Leu Val |
| | | Asp Gly Val Pro Ser Arg Phe Ser Gly Ser Gly |
| | | Ser Gly Thr Asp Phe Thr Leu Thr Ile Ser Ser |
| | | Leu Gln Pro Glu Asp Phe Ala Thr Tyr Tyr Cys |
| | | Val Gln Tyr Asp Glu Phe Pro Tyr Thr Phe Gly |
| | | Gly Gly Thr Lys Val Glu Ile Lys Arg Thr Val |
| | | Ala Ala Pro Ser Val Phe Ile Phe Pro Pro Ser |
| | | Asp Glu Gln Leu Lys Ser Gly Thr Ala Ser Val |
| | | Val Cys Leu Leu Asn Asn Phe Tyr Pro Arg Glu |
| | | Ala Lys Val Gln Trp Lys Val Asp Asn Ala Leu |
| | | Gln Ser Gly Asn Ser Gln Glu Ser Val Thr Glu |
| | | Gln Asp Ser Lys Asp Ser Thr Tyr Ser Leu Ser |
| | | Ser Thr Leu Thr Leu Ser Lys Ala Asp Tyr Glu |
| | | Lys His Lys Val Tyr Ala Cys Glu Val Thr His |
| | | Gln Gly Leu Ser Ser Pro Val Thr Lys Ser Phe |
| | | Asn Arg Gly Glu |
| 51 | Heavy | Gln Val Gln Leu Val Gln Ser Gly Ala Glu Val |
| | chain | Lys Lys Pro Gly Ser Ser Val Lys Val Ser Cys |
| | (Antibody | Lys Ala Ser Gly Tyr Thr Phe Asp Arg Tyr Trp |
| | W20) | Ile Glu Trp Val Arg Gln Ala Pro Gly Gln Gly |
| | | Leu Glu Trp Met Gly Glu Ile Leu Pro Gly Ser |
| | | Gly Asp Ile Asn Tyr Asn Glu Lys Phe Lys Gly |
| | | Arg Val Thr Ile Thr Ala Asp Lys Ser Thr Ser |
| | | Thr Ala Tyr Met Glu Leu Ser Ser Leu Arg Ser |
| | | Glu Asp Thr Ala Val Tyr Tyr Cys Ala Arg Met |
| | | Tyr Tyr Asp Tyr Asp Gln Gly Phe Asp Tyr Trp |
| | | Gly Gln Gly Thr Leu Val Thr Val Ser Ser Ala |
| | | Ser Thr Lys Gly Pro Ser Val Phe Pro Leu Ala |
| | | Pro Ser Ser Lys Ser Thr Ser Gly Gly Thr Ala |
| | | Ala Leu Gly Cys Leu Val Lys Asp Tyr Phe Pro |
| | | Glu Pro Val Thr Val Ser Trp Asn Ser Gly Ala |
| | | Leu Thr Ser Gly Val His Thr Phe Pro Ala Val |
| | | Leu Gln Ser Ser Gly Leu Tyr Ser Leu Ser Ser |
| | | Val Val Thr Val Pro Ser Ser Ser Leu Gly Thr |
| | | Gln Thr Tyr Ile Cys Asn Val Asn His Lys Pro |
| | | Ser Asn Thr Lys Val Asp Lys Lys Val Glu Pro |
| | | Lys Ser Cys Asp Lys Thr His Thr Cys Pro Pro |
| | | Cys Pro Ala Pro Glu Leu Leu Gly Gly Pro Ser |
| | | Val Phe Leu Phe Pro Pro Lys Pro Lys Asp Thr |
| | | Leu Met Ile Ser Arg Thr Pro Glu Val Thr Cys |
| | | Val Val Val Asp Val Ser His Glu Asp Pro Glu |
| | | Val Lys Phe Asn Trp Tyr Val Asp Gly Val Glu |
| | | Val His Asn Ala Lys Thr Lys Pro Arg Glu Glu |
| | | Gln Tyr Asn Ser Thr Tyr Arg Val Val Ser Val |
| | | Leu Thr Val Leu His Gln Asp Trp Leu Asn Gly |
| | | Lys Glu Tyr Lys Cys Lys Val Ser Asn Lys Ala |
| | | Leu Pro Ala Pro Ile Glu Lys Thr Ile Ser Lys |
| | | Ala Lys Gly Gln Pro Arg Glu Pro Gln Val Tyr |
| | | Thr Leu Pro Pro Ser Arg Asp Glu Leu Thr Lys |
| | | Asn Gln Val Ser Leu Thr Cys Leu Val Lys Gly |
| | | Phe Tyr Pro Ser Asp Ile Ala Val Glu Trp Glu |
| | | Ser Asn Gly Gln Pro Glu Asn Asn Tyr Lys Thr |
| | | Thr Pro Pro Val Leu Asp Ser Asp Gly Ser Phe |
| | | Phe Leu Tyr Ser Lys Leu Thr Val Asp Lys Ser |
| | | Arg Trp Gln Gln Gly Asn Val Phe Ser Cys Ser |
| | | Val Met His Glu Ala Leu His Asn His Tyr Thr |
| | | Gln Lys Ser Leu Ser Leu Ser Pro Gly Lys |
| 52 | Light chain | Asp Ile Gln Met Thr Gln Ser Pro Ser Ser Leu |
| | (Antibody | Ser Ala Ser Val Gly Asp Arg Val Thr Ile Thr |
| | W20) | Cys Lys Phe Ser Gln Asp Ile Asp Arg Phe Leu |
| | | Ser Trp Phe Gln Gln Lys Pro Gly Lys Ala Pro |
| | | Lys Ser Leu Ile Tyr Arg Val Lys Arg Leu Val |
| | | Asp Gly Val Pro Ser Arg Phe Ser Gly Ser Gly |
| | | Ser Gly Thr Asp Phe Thr Leu Thr Ile Ser Ser |
| | | Leu Gln Pro Glu Asp Phe Ala Thr Tyr Tyr Cys |
| | | Val Gln Tyr Asp Glu Phe Pro Tyr Thr Phe Gly |
| | | Gly Gly Thr Lys Val Glu Ile Lys Arg Thr Val |
| | | Ala Ala Pro Ser Val Phe Ile Phe Pro Pro Ser |
| | | Asp Glu Gln Leu Lys Ser Gly Thr Ala Ser Val |
| | | Val Cys Leu Leu Asn Asn Phe Tyr Pro Arg Glu |
| | | Ala Lys Val Gln Trp Lys Val Asp Asn Ala Leu |
| | | Gln Ser Gly Asn Ser Gln Glu Ser Val Thr Glu |
| | | Gln Asp Ser Lys Asp Ser Thr Tyr Ser Leu Ser |
| | | Ser Thr Leu Thr Leu Ser Lys Ala Asp Tyr Glu |
| | | Lys His Lys Val Tyr Ala Cys Glu Val Thr His |
| | | Gln Gly Leu Ser Ser Pro Val Thr Lys Ser Phe |
| | | Asn Arg Gly Glu |
| 53 | Heavy | Gln Val Gln Leu Val Gln Ser Gly Ala Glu Val |
| | chain | Lys Lys Pro Gly Ser Ser Val Lys Val Ser Cys |
| | (Antibody | Lys Ala Ser Gly Tyr Thr Phe Asp Arg Tyr Trp |
| | U43) | Ile Glu Trp Val Arg Gln Ala Pro Gly Gln Gly |
| | | Leu Glu Trp Met Gly Glu Ile Leu Pro Gly Ser |
| | | Gly Asp Ile Asn Tyr Asn Glu Lys Phe Lys Gly |
| | | Arg Val Thr Ile Thr Ala Asp Lys Ser Thr Ser |
| | | Thr Ala Tyr Met Glu Leu Ser Ser Leu Arg Ser |
| | | Glu Asp Thr Ala Val Tyr Tyr Cys Ala Arg Met |
| | | Tyr Tyr Asp Tyr Asp Gln Gly Phe Ser Leu Trp |
| | | Gly Gln Gly Thr Leu Val Thr Val Ser Ser Ala |
| | | Ser Thr Lys Gly Pro Ser Val Phe Pro Leu Ala |
| | | Pro Ser Ser Lys Ser Thr Ser Gly Gly Thr Ala |
| | | Ala Leu Gly Cys Leu Val Lys Asp Tyr Phe Pro |
| | | Glu Pro Val Thr Val Ser Trp Asn Ser Gly Ala |
| | | Leu Thr Ser Gly Val His Thr Phe Pro Ala Val |
| | | Leu Gln Ser Ser Gly Leu Tyr Ser Leu Ser Ser |
| | | Val Val Thr Val Pro Ser Ser Ser Leu Gly Thr |
| | | Gln Thr Tyr Ile Cys Asn Val Asn His Lys Pro |
| | | Ser Asn Thr Lys Val Asp Lys Lys Val Glu Pro |
| | | Lys Ser Cys Asp Lys Thr His Thr Cys Pro Pro |
| | | Cys Pro Ala Pro Glu Leu Leu Gly Gly Pro Ser |
| | | Val Phe Leu Phe Pro Pro Lys Pro Lys Asp Thr |
| | | Leu Met Ile Ser Arg Thr Pro Glu Val Thr Cys |
| | | Val Val Val Asp Val Ser His Glu Asp Pro Glu |
| | | Val Lys Phe Asn Trp Tyr Val Asp Gly Val Glu |
| | | Val His Asn Ala Lys Thr Lys Pro Arg Glu Glu |
| | | Gln Tyr Asn Ser Thr Tyr Arg Val Val Ser Val |
| | | Leu Thr Val Leu His Gln Asp Trp Leu Asn Gly |
| | | Lys Glu Tyr Lys Cys Lys Val Ser Asn Lys Ala |
| | | Leu Pro Ala Pro Ile Glu Lys Thr Ile Ser Lys |
| | | Ala Lys Gly Gln Pro Arg Glu Pro Gln Val Tyr |
| | | Thr Leu Pro Pro Ser Arg Asp Glu Leu Thr Lys |
| | | Asn Gln Val Ser Leu Thr Cys Leu Val Lys Gly |
| | | Phe Tyr Pro Ser Asp Ile Ala Val Glu Trp Glu |
| | | Ser Asn Gly Gln Pro Glu Asn Asn Tyr Lys Thr |
| | | Thr Pro Pro Val Leu Asp Ser Asp Gly Ser Phe |
| | | Phe Leu Tyr Ser Lys Leu Thr Val Asp Lys Ser |
| | | Arg Trp Gln Gln Gly Asn Val Phe Ser Cys Ser |
| | | Val Met His Glu Ala Leu His Asn His Tyr Thr |
| | | Gln Lys Ser Leu Ser Leu Ser Pro Gly Lys |
| 54 | Light chain | Asp Ile Gln Met Thr Gln Ser Pro Ser Ser Leu |
| | (Antibody | Ser Ala Ser Val Gly Asp Arg Val Thr Ile Thr |
| | U43) | Cys Lys Ala Ser Gln Asp Ile Asp Arg Phe Leu |
| | | Ser Trp Phe Gln Gln Lys Pro Lys Ala Pro Lys |
| | | Ser Leu Ile Tyr Arg Val Lys Arg Leu Val Asp |
| | | Gly Val Pro Ser Arg Phe Ser Gly Ser Gly Ser |
| | | Gly Thr Asp Phe Thr Leu Thr Ile Ser Ser Leu |
| | | Gln Pro Glu Asp Phe Ala Thr Tyr Tyr Cys Val |
| | | Gln Tyr Asp Glu Phe Pro Tyr Thr Phe Gly Gly |
| | | Gly Thr Lys Val Glu Ile Lys Arg Thr Val Ala |
| | | Ala Pro Ser Val Phe Ile Phe Pro Pro Ser Asp |
| | | Glu Gln Leu Lys Ser Gly Thr Ala Ser Val Val |
| | | Cys Leu Leu Asn Asn Phe Tyr Pro Arg Glu Ala |
| | | Lys Val Gln Trp Lys Val Asp Asn Ala Leu Gln |
| | | Ser Gly Asn Ser Gln Glu Ser Val Thr Glu Gln |
| | | Asp Ser Lys Asp Ser Thr Tyr Ser Leu Ser Ser |
| | | Thr Leu Thr Leu Ser Lys Ala Asp Tyr Glu Lys |
| | | His Lys Val Tyr Ala Cys Glu Val Thr His Gln |
| | | Gly Leu Ser Ser Pro Val Thr Lys Ser Phe Asn |
| | | Arg Gly Glu Cys |
| 55 | VH (DNA | ggatccactg gtcaggtgca gctggtgcag tctggcgctg |
| | sequence) | aggtgaagaa gcctggctcc tccgtgaagg tctcctgcaa |
| | (Antibody | ggcttctggc tacacattcg accgctattg gatcgagtgg |
| | W13) | gtgcgccagg cccctggcca aggcctggag tggatgggcg |
| | | agattctgcc tggcagcggc gacattaact acaatgagaa |
| | | gttcaagggc cgcgtcacga ttaccgegga caaatccacg |
| | | agcacagcct acatggagct gagcagcctg cgctctgagg |
| | | acacggccgt gtattactgt gcgcgcatgt actatgatta |
| | | cgaccagggc tttgactact ggggccaggg caccctggtc |
| | | accgtctcct ccgcctccac caagggccca tcggtcttcc |
| | | cgctagc |
| 56 | VH (DNA | ggatccactg gtcaggtgca gctggtgcag tctggcgctg |
| | sequence) | aggtgaagaa gcctggctcc tccgtgaagg tctcctgcaa |
| | (Antibody | ggcttctggc tacacattcg accgctattg gatcgagtgg |
| | W17) | gtgcgccagg cccctggcca aggcctggag tggatgggcg |
| | | agattctgcc tggcagcggc gacattaact acaatgagaa |
| | | gttcaagggc cgcgtcacga ttaccgcgga caaatccacg |
| | | agcacagcct acatggagct gagcagcctg cgctctgagg |
| | | acacggccgt gtattactgt gcgcgcatgt actatgatta |
| | | cgaccagggc tttgacctgt ggggccaggg caccctggtc |
| | | accgtctcct ccgcctccac caagggccca tcggtcttcc |
| | | cgctagc |
| 57 | VH (DNA | ggatccactg gtcaggtgca gctggtgcag tctggcgctg |
| | sequence) | aggtgaagaa gcctggctcc tccgtgaagg tctcctgcaa |
| | (Antibody | ggcttctggc tacacattcg accgctattg gatcgagtgg |
| | W18) | gtgcgccagg cccctggcca aggcctggag tggatgggcg |
| | | agattctgcc tggcageggc accattaact acaatgagaa |
| | | gttcaagggc cgcgtcacga ttaccgcgga caaatccacg |
| | | agcacagcct acatggagct gagcagcctg cgctctgagg |
| | | acacggccgt gtattactgt gcgcgcatgt actatgatta |
| | | cgaccagggc tttgacaact ggggccaggg caccctggtc |
| | | accgtctcct ccgcctccac caagggccca tcggtcttcc |
| | | cgctagc |
| 58 | VH (DNA | ggatccactg gtcaggtgca gctggtgcag tctggcgctg |
| | sequence) | aggtgaagaa gcctggctcc tccgtgaagg tctcctgcaa |
| | (Antibody | ggcttctggc tacacattcg accgctattg gatcgagtgg |
| | U43) | gtgcgccagg cccctggcca aggcctggag tggatgggcg |
| | | agattctgcc tggcagcggc gacattaact acaatgagaa |
| | | gttcaagggc cgcgtcacga ttaccgcgga caaatccacg |
| | | agcacagcct acatggagct gagcagcctg cgctctgagg |
| | | acacggccgt gtattactgt gcgcgcatgt actatgatta |
| | | cgaccagggc tttagcctgt ggggccaggg caccctggtc |
| | | accgtctcct ccgcctccac caagggccca tcggtcttcc |
| | | cgctagc |
| 59 | VH (DNA | ggatccactg gtcaggtgca gctggtgcag tctggcgctg |
| | sequence) | aggtgaagaa gcctggctcc tccgtgaagg tctcctgcaa |
| | (Antibody | ggcttctggc tacacattcg accgctattg gatcgagtgg |
| | W20) | gtgcgccagg cccctggcca aggcctggag tggatgggcg |
| | | agattctgcc tggcagcggc gacattaact acaatgagaa |
| | | gttcaagggc cgcgtcacga ttaccgcgga caaatccacg |
| | | agcacagcct acatggagct gagcagectg cgctctgagg |
| | | acacggccgt gtattactgt gcgcgcatgt actatgatta |
| | | cgaccagggc tttgactact ggggccaggg caccctggtc |
| | | accgtctcct ccgcctccac caagggccca tcggtcttcc |
| | | cgctagc |
| 60 | VL (DNA | gacatccaga tgacccagtc tccatcctcc ctgtctgcat |
| | sequence) | ctgtgggcga ccgcgtcacc atcacttgta agttcagtca |
| | (Antibody | ggacattgat cgcttcctga cctggtttca gcagaaacca |
| | W13) | ggcaaagccc ctaagtccct gatctatcgc gtgaagcgcc |
| | | tggtggatgg cgtcccatcc cgcttcagcg gcagtggctc |
| | | tggcacagat ttcactctca ccatcagcag cctgcagcct |
| | | gaagattttg caacttatta ctgcatccag tatgatgagt |
| | | ttccgtacac cttcggcggc ggcaccaagg tggagatcaa a |
| 61 | VL (DNA | gacatccaga tgacccagtc tccatcctcc ctgtctgcat |
| | sequence) | ctgtgggcga ccgcgtcacc atcacttgta agttcagtca |
| | (Antibody | ggacattgat cgcttcctga gctggtttca gcagaaacca |
| | W17) | ggcaaagccc ctaagtccct gatctatcgc gtgaagcgcc |
| | | tggtggatgg cgtcccatcc cgcttcagcg gcagtggctc |
| | | tggcacagat ttcactctca ccatcagcag cctgcagcct |
| | | gaagattttg caacttatta ctgcgttcag tatgatgagt |
| | | ttccgtacgg tttcggcggc ggcaccaagg tggagatcaa a |
| 62 | VL (DNA | gacatccaga tgacccagtc tccatcctcc ctgtctgcat |
| | sequence) | ctgtgggcga ccgcgtcacc atcacttgta agttcagtca |
| | (Antibody | ggacattgat cgcttcctga gctggtttca gcagaaacca |
| | W18) | ggcaaagccc ctaagtccct gatctatcgc gtgaagcgcc |
| | | tggtggatgg cgtcccatcc cgcttcagcg gcagtggctc |
| | | tggcacagat ttcactctca ccatcagcag cctgcagcct |
| | | gaagattttg caacttatta ctgcgttcag tatgatgagt |
| | | ttccgtacac cttcggcggc ggcaccaagg tggagatcaa a |
| 63 | VL (DNA | gacatccaga tgacccagtc tccatcctcc ctgtctgcat |
| | sequence) | ctgtgggcga ccgcgtcacc atcacttgta agttcagtca |
| | (Antibody | ggacattgat cgcttcctga gctggtttca gcagaaacca |
| | W20) | ggcaaagccc ctaagtccct gatctatcgc gtgaagcgcc |
| | | tggtggatgg cgtcccatcc cgcttcagcg gcagtggctc |
| | | tggcacagat ttcactctca ccatcagcag cctgcagcct |
| | | gaagattttg caacttatta ctgcgttcag tatgatgagt |
| | | ttccgtacac cttcggcggc ggcaccaagg tggagatcaa a |
| 64 | VL (DNA | gacatccaga tgacccagtc tccatcctcc ctgtctgcat |
| | sequence) | ctgtgggcga ccgcgtcacc atcacttgta aggcgagtca |
| | (Antibody | ggacattgat cgcttcctga gctggtttca gcagaaacca |
| | U43) | ggcaaagccc ctaagtccct gatctatcgc gtgaagcgcc |
| | | tggtggatgg cgtcccatcc cgcttcagcg gcagtggctc |
| | | tggcacagat ttcactctca ccatcagcag cctgcagcct |
| | | gaagattttg caacttatta ctgcgttcag tatgatgagt |
| | | ttccgtacac cttcggcggc ggcaccaagg tggagatcaa a |
| 65 | IgG1 heavy | tccaccaagg gcccatcggt cttcccgcta gcaccctcct |
| | chain | ccaagagcac ctctgggggc acagcggccc tgggctgcct |
| | constant | ggtcaaggac tacttccccg aaccggtgac ggtgtcgtgg |
| | region | aactcaggcg ccctgaccag cggcgtgcac accttcccgg |
| | (DNA | ctgtcctaca gtcctcagga ctctactccc tcagcagcgt |
| | sequence) | ggtgaccgtg ccctccagca gcttgggcac ccagacctac |
| | | atctgcaacg tgaatcacaa gcccagcaac accaaggtgg |
| | | acaagaaagt tgagcccaaa tottgtgaca aaactcacac |
| | | atgcccaccg tgcccagcac ctgaactect ggggggaccg |
| | | tcagtcttcc tottcccccc aaaacccaag gacaccctca |
| | | tgatctcccg gacccctgag gtcacatgcg tggtggtgga |
| | | cgtgagccac gaagaccctg aggtcaagtt caactggtac |
| | | gtggacggcg tggaggtgca taatgccaag acaaagccgc |
| | | gggaggagca gtacaacagc acgtaccgtg tggtcagcgt |
| | | cctcaccgtc ctgcaccagg actggctgaa tggcaaggag |
| | | tacaagtgca aggtctccaa caaagccctc ccagccccca |
| | | tcgagaaaac catctccaaa gccaaagggc agccccgaga |
| | | accacaggtg tacaccctgc ccccatcccg ggacgagctg |
| | | accaagaacc aggtcagcct gacctgcctg gtcaaaggct |
| | | tctatcccag cgacatcgcc gtggagtggg agagcaatgg |
| | | gcagccggag aacaactaca agaccacgcc ccccgtgctg |
| | | gactccgacg gctccttctt cctctatagc aagctcaccg |
| | | tggacaagag caggtggcag caggggaacg tcttctcatg |
| | | ctccgtgatg catgaggctc tgcacaacca ctacacgcag |
| | | aagagcctct ccctgtctcc gggtaaatga |
| 66 | IgG4 heavy | ctagcgccct gctccaggag cacctccgag agcacagccg |
| | chain | ccctgggctg cctggtcaag gactacttcc ccgaaccggt |
| | constant | gacggtgtcg tggaactcag gcgccctgac cagcggcgtg |
| | region | cacaccttcc cggctgtect acagtcctca ggactctact |
| | (DNA | ccctcagcag cgtggtgacc gtgccctcca gcagcttggg |
| | sequence) | cacgaagacc tacacctgca acgtagatca caagcccagc |
| | | aacaccaagg tggacaagag agttgagtcc aaatatggtc |
| | | ccccatgccc accctgccca gcacctgagt tcctgggggg |
| | | accatcagtc ttcctgttcc ccccaaaacc caaggacact |
| | | ctcatgatct cccggacccc tgaggtcacg tgcgtggtgg |
| | | tggacgtgag ccaggaagac cccgaggtcc agttcaactg |
| | | gtacgtggat ggcgtggagg tgcataatgc caagacaaag |
| | | ccgcgggagg agcagttcaa cagcacgtac cgtgtggtca |
| | | gcgtcctcac cgtcctgcac caggactggc tgaacggcaa |
| | | ggagtacaag tgcaaggtct ccaacaaagg cctcccgtcc |
| | | tccatcgaga aaaccatctc caaagccaaa gggcagcccc |
| | | gagagccaca ggtgtacacc ctgcccccat cccaggagga |
| | | gatgaccaag aaccaggtca gcctgacctg cctggtcaaa |
| | | ggcttctacc ccagcgacat cgccgtggag tgggagagca |
| | | atgggcagcc ggagaacaac tacaagacca cgcctcccgt |
| | | gctggactcc gacggctcct tcttcctcta cagcaggcta |
| | | accgtggaca agagcaggtg gcaggagggg aatgtcttct |
| | | catgctccgt gatgcatgag gctctgcaca accactacac |
| | | acagaagagc ctctccctgt ctctgggtaa at |
| 67 | kappa light | cgaactgtgg ctgcaccatc tgtcttcatc ttcccgccat |
| | chain | ctgatgagca gttgaaatct ggaactgcct ctgttgtgtg |
| | region | cctgctgaat aacttctatc ccagagaggc caaagtacag |
| | (DNA | tggaaggtgg ataacgccct ccaatcgggt aactcccagg |
| | sequence) | agagtgtcac agagcaggac agcaaggaca gcacctacag |
| | | cctcagcagc accctgacgc tgagcaaagc agactacgag |
| | | aaacacaaag tctacgcctg cgaagtcacc catcagggcc |
| | | tgagctcgcc cgtcacaaag agcttcaaca ggggagagtg |
| | | ctaa |
| 68 | Heavy | Met Glu Thr Asp Thr Leu Leu Leu Trp Val Leu |
| | chain | Leu Leu Trp Val Pro Gly Ser Thr Gly Gln Val |
| | (Antibody | Gln Leu Val Gln Ser Gly Ala Glu Val Lys Lys |
| | W17) with | Pro Gly Ser Ser Val Lys Val Ser Cys Lys Ala |
| | leader | Ser Gly Tyr Thr Phe Asp Arg Tyr Trp Ile Glu |
| | sequence | Trp Val Arg Gln Ala Pro Gly Gln Gly Leu Glu |
| | | Trp Met Gly Glu Ile Leu Pro Gly Ser Gly Asp |
| | | Ile Asn Tyr Asn Glu Lys Phe Lys Gly Arg Val |
| | | Thr Ile Thr Ala Asp Lys Ser Thr Ser Thr Ala |
| | | Tyr Met Glu Leu Ser Ser Leu Arg Ser Glu Asp |
| | | Thr Ala Val Tyr Tyr Cys Ala Arg Met Tyr Tyr |
| | | Asp Tyr Asp Gln Gly Phe Asp Leu Trp Gly Gln |
| | | Gly Thr Leu Val Thr Val Ser Ser Ala Ser Thr |
| | | Lys Gly Pro Ser Val Phe Pro Leu Ala Pro Cys |
| | | Ser Arg Ser Thr Ser Glu Ser Thr Ala Ala Leu |
| | | Gly Cys Leu Val Lys Asp Tyr Phe Pro Glu Pro |
| | | Val Thr Val Ser Trp Asn Ser Gly Ala Leu Thr |
| | | Ser Gly Val His Thr Phe Pro Ala Val Leu Gln |
| | | Ser Ser Gly Leu Tyr Ser Leu Ser Ser Val Val |
| | | Thr Val Pro Ser Ser Ser Leu Gly Thr Lys Thr |
| | | Tyr Thr Cys Asn Val Asp His Lys Pro Ser Asn |
| | | Thr Lys Val Asp Lys Arg Val Glu Ser Lys Tyr |
| | | Gly Pro Pro Cys Pro Pro Cys Pro Ala Pro Glu |
| | | Phe Leu Gly Gly Pro Ser Val Phe Leu Phe Pro |
| | | Pro Lys Pro Lys Asp Thr Leu Met Ile Ser Arg |
| | | Thr Pro Glu Val Thr Cys Val Val Val Asp Val |
| | | Ser Gln Glu Asp Pro Glu Val Gln Phe Asn Trp |
| | | Tyr Val Asp Gly Val Glu Val His Asn Ala Lys |
| | | Thr Lys Pro Arg Glu Glu Gln Phe Asn Ser Thr |
| | | Tyr Arg Val Val Ser Val Leu Thr Val Leu His |
| | | Gln Asp Trp Leu Asn Gly Lys Glu Tyr Lys Cys |
| | | Lys Val Ser Asn Lys Gly Leu Pro Ser Ser Ile |
| | | Glu Lys Thr Ile Ser Lys Ala Lys Gly Gln Pro |
| | | Arg Glu Pro Gln Val Tyr Thr Leu Pro Pro Ser |
| | | Gln Glu Glu Met Thr Lys Asn Gln Val Ser Leu |
| | | Thr Cys Leu Val Lys Gly Phe Tyr Pro Ser Asp |
| | | Ile Ala Val Glu Trp Glu Ser Asn Gly Gln Pro |
| | | Glu Asn Asn Tyr Lys Thr Thr Pro Pro Val Leu |
| | | Asp Ser Asp Gly Ser Phe Phe Leu Tyr Ser Arg |
| | | Leu Thr Val Asp Lys Ser Arg Trp Gln Glu Gly |
| | | Asn Val Phe Ser Cys Ser Val Met His Glu Ala |
| | | Leu His Asn His Tyr Thr Gln Lys Ser Leu Ser |
| | | Leu Ser Leu Gly Lys |
| 69 | Light chain | Asp Ile Gln Met Thr Gln Ser Pro Ser Ser Leu |
| | variable | Ser Ala Ser Val Gly Asp Arg Val Thr Ile Thr |
| | region (VL) | Cys Lys Phe Ser Gln Asp Ile Asp Arg Phe Leu |
| | (Antibody | Ser Trp Phe Gln Gln Lys Pro Gly Lys Ala Pro |
| | W17) | Lys Ser Leu Ile Tyr Arg Val Lys Arg Leu Val |
| | | Asp Gly Val Pro Ser Arg Phe Ser Gly Ser Gly |
| | | Ser Gly Thr Asp Phe Thr Leu Thr Ile Ser Ser |
| | | Leu Gln Pro Glu Asp Phe Ala Thr Tyr Tyr Cys |
| | | Val Gln Tyr Asp Glu Phe Pro Tyr Gly Phe Gly |
| | | Gly Gly Thr Lys Val Glu Ile Lys |
| 70 | Heavy | Gln Val Gln Leu Val Gln Ser Gly Ala Glu Val |
| | chain | Lys Lys Pro Gly Ser Ser Val Lys Val Ser Cys |
| | variable | Lys Ala Ser Gly Tyr Thr Phe Asp Arg Tyr Trp |
| | region (VH) | Ile Glu Trp Val Arg Gln Ala Pro Gly Gln Gly |
| | (Antibody | Leu Glu Trp Met Gly Glu Ile Leu Pro Gly Ser |
| | W17) | Gly Asp Ile Asn Tyr Asn Glu Lys Phe Lys Gly |
| | | Arg Val Thr Ile Thr Ala Asp Lys Ser Thr Ser |
| | | Thr Ala Tyr Met Glu Leu Ser Ser Leu Arg Ser |
| | | Glu Asp Thr Ala Val Tyr Tyr Cys Ala Arg Met |
| | | Tyr Tyr Asp Tyr Asp Gln Gly Phe Asp Leu Trp |
| | | Gly Gln Gly Thr Leu Val Thr Val Ser Ser |
| 71 | VH CDR1 | Gly Tyr Thr Phe Asp Arg Tyr |
| 72 | VH CDR2 | Leu Pro Gly Ser Gly Asp |
| 73 | Heavy | Gln Val Gln Leu Val Gln Ser Gly Ala Glu Val |
| | chain | Lys Lys Pro Gly Ser Ser Val Lys Val Ser Cys |
| | (Antibody | Lys Ala Ser Gly Tyr Thr Phe Asp Arg Tyr Trp |
| | W17) | Ile Glu Trp Val Arg Gln Ala Pro Gly Gln Gly |
| | without | Leu Glu Trp Met Gly Glu Ile Leu Pro Gly Ser |
| | leader | Gly Asp Ile Asn Tyr Asn Glu Lys Phe Lys Gly |
| | sequence | Arg Val Thr Ile Thr Ala Asp Lys Ser Thr Ser |
| | | Thr Ala Tyr Met Glu Leu Ser Ser Leu Arg Ser |
| | | Glu Asp Thr Ala Val Tyr Tyr Cys Ala Arg Met |
| | | Tyr Tyr Asp Tyr Asp Gln Gly Phe Asp Leu Trp |
| | | Gly Gln Gly Thr Leu Val Thr Val Ser Ser Ala |
| | | Ser Thr Lys Gly Pro Ser Val Phe Pro Leu Ala |
| | | Pro Cys Ser Arg Ser Thr Ser Glu Ser Thr Ala |
| | | Ala Leu Gly Cys Leu Val Lys Asp Tyr Phe Pro |
| | | Glu Pro Val Thr Val Ser Trp Asn Ser Gly Ala |
| | | Leu Thr Ser Gly Val His Thr Phe Pro Ala Val |
| | | Leu Gln Ser Ser Gly Leu Tyr Ser Leu Ser Ser |
| | | Val Val Thr Val Pro Ser Ser Ser Leu Gly Thr |
| | | Lys Thr Tyr Thr Cys Asn Val Asp His Lys Pro |
| | | Ser Asn Thr Lys Val Asp Lys Arg Val Glu Ser |
| | | Lys Tyr Gly Pro Pro Cys Pro Pro Cys Pro Ala |
| | | Pro Glu Phe Leu Gly Gly Pro Ser Val Phe Leu |
| | | Phe Pro Pro Lys Pro Lys Asp Thr Leu Met Ile |
| | | Ser Arg Thr Pro Glu Val Thr Cys Val Val Val |
| | | Asp Val Ser Gln Glu Asp Pro Glu Val Gln Phe |
| | | Asn Trp Tyr Val Asp Gly Val Glu Val His Asn |
| | | Ala Lys Thr Lys Pro Arg Glu Glu Gln Phe Asn |
| | | Ser Thr Tyr Arg Val Val Ser Val Leu Thr Val |
| | | Leu His Gln Asp Trp Leu Asn Gly Lys Glu Tyr |
| | | Lys Cys Lys Val Ser Asn Lys Gly Leu Pro Ser |
| | | Ser Ile Glu Lys Thr Ile Ser Lys Ala Lys Gly |
| | | Gln Pro Arg Glu Pro Gln Val Tyr Thr Leu Pro |
| | | Pro Ser Gln Glu Glu Met Thr Lys Asn Gln Val |
| | | Ser Leu Thr Cys Leu Val Lys Gly Phe Tyr Pro |
| | | Ser Asp Ile Ala Val Glu Trp Glu Ser Asn Gly |
| | | Gln Pro Glu Asn Asn Tyr Lys Thr Thr Pro Pro |
| | | Val Leu Asp Ser Asp Gly Ser Phe Phe Leu Tyr |
| | | Ser Arg Leu Thr Val Asp Lys Ser Arg Trp Gln |
| | | Glu Gly Asn Val Phe Ser Cys Ser Val Met His |
| | | Glu Ala Leu His Asn His Tyr Thr Gln Lys Ser |
| | | Leu Ser Leu Ser Leu Gly Lys |

1. A method of treating hidradenitis suppurativa, comprising administering an effective amount of an anti-IL-1β antibody to a human subject in need thereof, wherein the anti-IL-1β antibody comprises:
i) (a) HCDR1 comprising the amino acid sequence of SEQ ID NO: 10; (b) HCDR2 comprising the amino acid sequence of SEQ ID NO: 21; (c) HCDR3 comprising the amino acid sequence of SEQ ID NO: 38; (d) LCDR1 comprising the amino acid sequence of SEQ ID NO: 39; (e) LCDR2 comprising the amino acid sequence of SEQ ID NO: 2; and (f) LCDR3 comprising the amino acid sequence of SEQ ID NO: 41; or
ii) (a) HCDR1 comprising the amino acid sequence of SEQ ID NO: 71; (b) HCDR2 comprising the amino acid sequence of SEQ ID NO: 72; (c) HCDR3 comprising the amino acid sequence of SEQ ID NO: 38; (d) LCDR1 comprising the amino acid sequence of SEQ ID NO: 39; (e) LCDR2 comprising the amino acid sequence of SEQ ID NO: 2; and (f) LCDR3 comprising the amino acid sequence of SEQ ID NO: 41.

2. (canceled)

3. (canceled)

4. (canceled)

5. The method of claim 1, wherein the anti-IL-1β antibody comprises:
i) the CDRs of claim 1. i) and further comprises a light chain variable region (VL) comprising an amino acid sequence that is at least 90%, 91%, 92%, 93%, 94%, 95%, 96%, 97%, 98%, or 99% identical to the amino acid sequence of SEQ ID NO: 69; or
ii) the CDRS of claim 1. ii) and further comprises a VL comprising an amino acid sequence that is at least 90%, 91%, 92%, 93%, 94%, 95%, 96%, 97%, 98%, or 99% identical to the amino acid sequence of SEQ ID NO: 69.

6. The method of claim 1, wherein the anti-IL-1β antibody comprises:
i) the CDRs of claim 1. i) and further comprises a VL comprising the amino acid sequence of SEQ ID NO: 69; or
ii) the CDRS of claim 1. ii) and further comprises a VL comprising the amino acid sequence of SEQ ID NO: 69.

7. The method of claim 1, wherein the anti-IL-1β antibody comprises a heavy chain variable region (VH) and a light chain variable region (VL), wherein:
the VH is at least 90%, 91%, 92%, 93%, 94%, 95%, 96%, 97%, 98%, 99%, or 100% identical to the amino acid sequence of SEQ ID NO: 70 and the VL is at least 90%, 91%, 92%, 93%, 94%, 95%, 96%, 97%, 98%, 99%, or 100% identical to the amino acid sequence of SEQ ID NO: 69.

8. The method of claim 1, wherein the anti-IL-1β antibody comprises a heavy chain variable region (VH) and a light chain variable region (VL), wherein the VH comprises the amino acid sequence of SEQ ID NO: 70 and the VL comprises the amino acid sequence of SEQ ID NO: 69.

9. (canceled)

10. A method of treating hidradenitis suppurativa comprising administering an effective amount of an anti-IL-1β antibody to a human subject in need thereof, wherein the anti-IL-1β antibody thereof comprises a light chain variable region (VL) comprising the amino acid sequence of SEQ ID NO: 69, and a heavy chain variable region (VH) comprising the amino acid sequence of SEQ ID NO: 70.

11. (canceled)

12. (canceled)

13. (canceled)

14. (canceled)

15. (canceled)

16. (canceled)

17. (canceled)

18. The method of claim 1, wherein the anti-IL-1β antibody: (a) is an antibody fragment: or (b) comprises a human IgG4 heavy chain constant region and a human IgG kappa light chain constant region.

19. (canceled)

20. The method of claim 1, wherein the anti-IL-1β antibody is a humanized antibody.

21. (canceled)

22. (canceled)

23. The method of claim 1, wherein the anti-IL-1β antibody comprises a light chain (LC) comprising an amino acid sequence having at least 85%, 90%, 91%, 92%, 93%, 94%, 95%, 96%, 97%, 98%, 99%, or 100% sequence identity to the amino acid sequence of SEQ ID NO: 48, and a heavy chain (HC) sequence comprising an amino acid having at least 85%, 90%, 91%, 92%, 93%, 94%, 95%, 96%, 97%, 98%, 99%, or 100% sequence identity to the amino acid sequence of SEQ ID NO: 68 or SEQ ID NO: 73.

24. (canceled)

25. The method of claim 1, wherein the anti-IL-1β antibody comprises a light chain (LC) comprising the amino acid sequence of SEQ ID NO: 48 and a heavy chain (HC) comprising the amino acid sequence of SEQ ID NO: 68.

26. (canceled)

27. The method of claim 10, wherein the anti-IL-1β antibody is administered subcutaneously.

28. The method of claim 10, wherein a loading dose of 600 mg of the anti-IL-1β antibody is administered followed by a dose of 300 mg of the anti-IL-1β antibody every four weeks (Q4W).

29. The method of claim 10, wherein a loading dose of 300 mg of the anti-IL-1β antibody is administered followed by a dose of 150 mg of the anti-IL-1β antibody every two weeks (Q2W).

30. The method of claim 10, wherein the subject has moderate to severe hidradenitis suppurativa.

31. The method of claim 10, wherein the subject is an adult.

32. (canceled)

33. (canceled)

34. (canceled)

35. (canceled)

36. (canceled)

37. The method of claim 10, wherein the subject has a Numerical Rating Scale (NRS) in Patient's Global Assessment of Skin Pain (PGA Skin Pain) ≥3 at baseline.

38. (canceled)

39. (canceled)

40. (canceled)

41. (canceled)

42. The method of claim 10, wherein: (i) the subject has failed treatment with an anti-TNF therapy; or (ii) anti-TNF therapy is contraindicated for the subject.

43. (canceled)

44. (canceled)

45. The method of claim 1, wherein the anti-IL-1β antibody comprises a light chain (LC) comprising the amino acid sequence of SEQ ID NO: 48 and a heavy chain (HC) comprising the amino acid sequence of SEQ ID NO: 73.

46. The method of claim 28, wherein the first 300 mg dose is administered four weeks after the loading dose.

47. The method of claim 29, wherein the first 150 mg dose is administered two weeks after the loading dose.


SEQUENCE LISTING (73 sequences)

SEQ ID NO: 1 - protein, 11 aa, unidentified
  source 1..11: note = Murine
KASQDIDRYLS

SEQ ID NO: 2 - protein, 7 aa, unidentified
  source 1..7: note = Murine
RVKRLVD

SEQ ID NO: 3 - protein, 9 aa, unidentified
  source 1..9: note = Murine
LQYDEFPYT

SEQ ID NO: 4 - protein, 10 aa, unidentified
  source 1..10: note = Murine
GYTFSRYWIE

SEQ ID NO: 5 - protein, 17 aa, unidentified
  source 1..17: note = Murine
EILPGNGNINYNEKFKG

SEQ ID NO: 6 - protein, 11 aa, unidentified
  source 1..11: note = Murine
IYYDYDQGFTY

SEQ ID NO: 7 - protein, 9 aa, synthetic construct
VQYDEFNYT

SEQ ID NO: 8 - protein, 17 aa, synthetic construct
EILPGTGTINYNEKFKG

SEQ ID NO: 9 - protein, 11 aa, synthetic construct
VYYDYDYGFDY

SEQ ID NO: 10 - protein, 10 aa, synthetic construct
GYTFDRYWIE

SEQ ID NO: 11 - protein, 11 aa, synthetic construct
VYYDYDYGFDL

SEQ ID NO: 12 - protein, 11 aa, synthetic construct
KASQDIDRYLT

SEQ ID NO: 13 - protein, 9 aa, synthetic construct
IQYDEFNYT

SEQ ID NO: 14 - protein, 11 aa, synthetic construct
VYYDYDYGFDN

SEQ ID NO: 15 - protein, 9 aa, synthetic construct
IQYDEFPYT

SEQ ID NO: 16 - protein, 17 aa, synthetic construct
EILPGSGTINYNEKFKG

SEQ ID NO: 17 - protein, 9 aa, synthetic construct
VQYDEFPYT

SEQ ID NO: 18 - protein, 17 aa, synthetic construct
EILPGTGDINYNEKFKG

SEQ ID NO: 19 - protein, 11 aa, synthetic construct
VYYDYDYGFTL

SEQ ID NO: 20 - protein, 9 aa, synthetic construct
IQYDEFPYL

SEQ ID NO: 21 - protein, 17 aa, synthetic construct
EILPGSGDINYNEKFKG

SEQ ID NO: 22 - protein, 11 aa, synthetic construct
KFSQDIDRYLT

SEQ ID NO: 23 - protein, 17 aa, synthetic construct
EILPGSGNINYNEKFKG

SEQ ID NO: 24 - protein, 11 aa, synthetic construct
VYYDYDQGFDN

SEQ ID NO: 25 - protein, 9 aa, synthetic construct
VQYDEFPYL

SEQ ID NO: 26 - protein, 11 aa, synthetic construct
VYYDYDYGFTY

SEQ ID NO: 27 - protein, 11 aa, synthetic construct
VYYDYDYGFTN

SEQ ID NO: 28 - protein, 17 aa, synthetic construct
EILPGTGNINYNEKFKG

SEQ ID NO: 29 - protein, 11 aa, synthetic construct
KASQDIDRFLS

SEQ ID NO: 30 - protein, 11 aa, synthetic construct
MYYDYDQGFSL

SEQ ID NO: 31 - protein, 11 aa, synthetic construct
KASQDIDRFLT

SEQ ID NO: 32 - protein, 11 aa, synthetic construct
MYYDYDQGFTN

SEQ ID NO: 33 - protein, 11 aa, synthetic construct
KASQDIDRFLS

SEQ ID NO: 34 - protein, 9 aa, synthetic construct
VQYDEFAYT

SEQ ID NO: 35 - protein, 11 aa, synthetic construct
MYYDYDQGFDY

SEQ ID NO: 36 - protein, 11 aa, synthetic construct
MYYDYDQGFDN

SEQ ID NO: 37 - protein, 11 aa, synthetic construct
KFSQDIDRFLT

SEQ ID NO: 38 - protein, 11 aa, synthetic construct
MYYDYDQGFDL

SEQ ID NO: 39 - protein, 11 aa, synthetic construct
KFSQDIDRFLS

SEQ ID NO: 40 - protein, 11 aa, synthetic construct
MYYDYDQGFTL

SEQ ID NO: 41 - protein, 9 aa, synthetic construct
VQYDEFPYG

SEQ ID NO: 42 - protein, 11 aa, synthetic construct
MYYDYDQGFTY

SEQ ID NO: 43 - DNA, 387 bp, unidentified
  source 1..387: mol_type = genomic DNA; note = Murine
atggacatgaggacccctgctcagtttcttggaatctttttcttctggtttccaggtatcagatgtgacatcaagatgacccagtctccatcttccatgtatgcatctctaggagagagagtcactatcacttgcaaggcgagtcaggacattgataggtatttaagttggttccagcagaaaccagggaaatctcctaagaccctgatctatcgtgtaaagagattggtagatggggtcccatcaaggttcagtggcagcgcatctgggcaagattattctctcaccatcagcagcctgcagtatgaagatatgggaatttattattgtctacagtatgatgagtttccgtacacgttcggaggggggaccaagctggaaataaaa

SEQ ID NO: 44 - DNA, 417 bp, unidentified
  source 1..417: mol_type = genomic DNA; note = Murine
atggaatggacctgggtctttctcttcctcctgtcagtaactgcaggtgtccactcccaggttcagctgcagcagtctggagctgagctgatgaagcctggggcctcagtgaagatatcctgcaaggctactggctacacattcagtaggtattggatagagtggataaagcagaggcctggacatggccttgagtggattggagagattttacctggaaatggaaatattaactacaatgagaagttcaagggcaaggccacaatctctgcagattcttcctccgaaacagcctacatgcaactcagcagcctgtcctctgaggactctgccgtctattattgttcaacaatctactatgattacgaccaggggtttacttactggggccaagggactctggtcactgtttctgca

SEQ ID NO: 45 - protein, 450 aa, synthetic construct
QVQLVQSGAEVKKPGSSVKVSCKASGYTFDRYWIEWVRQAPGQGLEWMGEILPGSGDINYNEKFKGRVTITADKSTSTAYMELSSLRSEDTAVYYCARMYYDYDQGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 214 aa, synthetic construct
DIQMTQSPSSLSASVGDRVTITCKFSQDIDRFLTWFQQKPGKAPKSLIYRVKRLVDGVPSRFSGSGSGTDFTLTISSLQPEDFATYYCIQYDEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 450 aa, synthetic construct
QVQLVQSGAEVKKPGSSVKVSCKASGYTFDRYWIEWVRQAPGQGLEWMGEILPGSGDINYNEKFKGRVTITADKSTSTAYMELSSLRSEDTAVYYCARMYYDYDQGFDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 214 aa, synthetic construct
DIQMTQSPSSLSASVGDRVTITCKFSQDIDRFLSWFQQKPGKAPKSLIYRVKRLVDGVPSRFSGSGSGTDFTLTISSLQPEDFATYYCVQYDEFPYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 450 aa, synthetic construct
QVQLVQSGAEVKKPGSSVKVSCKASGYTFDRYWIEWVRQAPGQGLEWMGEILPGSGTINYNEKFKGRVTITADKSTSTAYMELSSLRSEDTAVYYCARMYYDYDQGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 213 aa, synthetic construct
DIQMTQSPSSLSASVGDRVTITCKFSQDIDRFLSWFQQKPGKAPKSLIYRVKRLVDGVPSRFSGSGSGTDFTLTISSLQPEDFATYYCVQYDEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 51 - protein, 450 aa, synthetic construct; identical to SEQ ID NO: 45

SEQ ID NO: 52 - protein, 213 aa, synthetic construct; identical to SEQ ID NO: 50

SEQ ID NO: 53 - protein, 450 aa, synthetic construct
QVQLVQSGAEVKKPGSSVKVSCKASGYTFDRYWIEWVRQAPGQGLEWMGEILPGSGDINYNEKFKGRVTITADKSTSTAYMELSSLRSEDTAVYYCARMYYDYDQGFSLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 213 aa, synthetic construct
DIQMTQSPSSLSASVGDRVTITCKASQDIDRFLSWFQQKPKAPKSLIYRVKRLVDGVPSRFSGSGSGTDFTLTISSLQPEDFATYYCVQYDEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - DNA, 407 bp, Homo sapiens
  source 1..407: mol_type = genomic DNA
ggatccactggtcaggtgcagctggtgcagtctggcgctgaggtgaagaagcctggctcctccgtgaaggtctcctgcaaggcttctggctacacattcgaccgctattggatcgagtgggtgcgccaggcccctggccaaggcctggagtggatgggcgagattctgcctggcagcggcgacattaactacaatgagaagttcaagggccgcgtcacgattaccgcggacaaatccacgagcacagcctacatggagctgagcagcctgcgctctgaggacacggccgtgtattactgtgcgcgcatgtactatgattacgaccagggctttgactactggggccagggcaccctggtcaccgtctcctccgcctccaccaagggcccatcggtcttcccgctagc

SEQ ID NO: 56 - DNA, 407 bp, Homo sapiens
  source 1..407: mol_type = genomic DNA
ggatccactggtcaggtgcagctggtgcagtctggcgctgaggtgaagaagcctggctcctccgtgaaggtctcctgcaaggcttctggctacacattcgaccgctattggatcgagtgggtgcgccaggcccctggccaaggcctggagtggatgggcgagattctgcctggcagcggcgacattaactacaatgagaagttcaagggccgcgtcacgattaccgcggacaaatccacgagcacagcctacatggagctgagcagcctgcgctctgaggacacggccgtgtattactgtgcgcgcatgtactatgattacgaccagggctttgacctgtggggccagggcaccctggtcaccgtctcctccgcctccaccaagggcccatcggtcttcccgctagc

SEQ ID NO: 57 - DNA, 407 bp, Homo sapiens
  source 1..407: mol_type = genomic DNA
ggatccactggtcaggtgcagctggtgcagtctggcgctgaggtgaagaagcctggctcctccgtgaaggtctcctgcaaggcttctggctacacattcgaccgctattggatcgagtgggtgcgccaggcccctggccaaggcctggagtggatgggcgagattctgcctggcagcggcaccattaactacaatgagaagttcaagggccgcgtcacgattaccgcggacaaatccacgagcacagcctacatggagctgagcagcctgcgctctgaggacacggccgtgtattactgtgcgcgcatgtactatgattacgaccagggctttgacaactggggccagggcaccctggtcaccgtctcctccgcctccaccaagggcccatcggtcttcccgctagc

SEQ ID NO: 58 - DNA, 407 bp, Homo sapiens
  source 1..407: mol_type = genomic DNA
ggatccactggtcaggtgcagctggtgcagtctggcgctgaggtgaagaagcctggctcctccgtgaaggtctcctgcaaggcttctggctacacattcgaccgctattggatcgagtgggtgcgccaggcccctggccaaggcctggagtggatgggcgagattctgcctggcagcggcgacattaactacaatgagaagttcaagggccgcgtcacgattaccgcggacaaatccacgagcacagcctacatggagctgagcagcctgcgctctgaggacacggccgtgtattactgtgcgcgcatgtactatgattacgaccagggctttagcctgtggggccagggcaccctggtcaccgtctcctccgcctccaccaagggcccatcggtcttcccgctagc

SEQ ID NO: 59 - DNA, 407 bp, Homo sapiens; identical to SEQ ID NO: 55
  source 1..407: mol_type = genomic DNA

SEQ ID NO: 60 - DNA, 321 bp, Homo sapiens
  source 1..321: mol_type = genomic DNA
gacatccagatgacccagtctccatcctccctgtctgcatctgtgggcgaccgcgtcaccatcacttgtaagttcagtcaggacattgatcgcttcctgacctggtttcagcagaaaccaggcaaagcccctaagtccctgatctatcgcgtgaagcgcctggtggatggcgtcccatcccgcttcagcggcagtggctctggcacagatttcactctcaccatcagcagcctgcagcctgaagattttgcaacttattactgcatccagtatgatgagtttccgtacaccttcggcggcggcaccaaggtggagatcaaa

SEQ ID NO: 61 - DNA, 321 bp, Homo sapiens
  source 1..321: mol_type = genomic DNA
gacatccagatgacccagtctccatcctccctgtctgcatctgtgggcgaccgcgtcaccatcacttgtaagttcagtcaggacattgatcgcttcctgagctggtttcagcagaaaccaggcaaagcccctaagtccctgatctatcgcgtgaagcgcctggtggatggcgtcccatcccgcttcagcggcagtggctctggcacagatttcactctcaccatcagcagcctgcagcctgaagattttgcaacttattactgcgttcagtatgatgagtttccgtacggtttcggcggcggcaccaaggtggagatcaaa

SEQ ID NO: 62 - DNA, 321 bp, Homo sapiens
  source 1..321: mol_type = genomic DNA
gacatccagatgacccagtctccatcctccctgtctgcatctgtgggcgaccgcgtcaccatcacttgtaagttcagtcaggacattgatcgcttcctgagctggtttcagcagaaaccaggcaaagcccctaagtccctgatctatcgcgtgaagcgcctggtggatggcgtcccatcccgcttcagcggcagtggctctggcacagatttcactctcaccatcagcagcctgcagcctgaagattttgcaacttattactgcgttcagtatgatgagtttccgtacaccttcggcggcggcaccaaggtggagatcaaa

SEQ ID NO: 63 - DNA, 321 bp, Homo sapiens; identical to SEQ ID NO: 62
  source 1..321: mol_type = genomic DNA

SEQ ID NO: 64 - DNA, 321 bp, Homo sapiens
  source 1..321: mol_type = genomic DNA
gacatccagatgacccagtctccatcctccctgtctgcatctgtgggcgaccgcgtcaccatcacttgtaaggcgagtcaggacattgatcgcttcctgagctggtttcagcagaaaccaggcaaagcccctaagtccctgatctatcgcgtgaagcgcctggtggatggcgtcccatcccgcttcagcggcagtggctctggcacagatttcactctcaccatcagcagcctgcagcctgaagattttgcaacttattactgcgttcagtatgatgagtttccgtacaccttcggcggcggcaccaaggtggagatcaaa

SEQ ID NO: 65 - DNA, 990 bp, Homo sapiens
  source 1..990: mol_type = genomic DNA
tccaccaagggcccatcggtcttcccgctagcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgccccccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 66 - DNA, 952 bp, Homo sapiens
  source 1..952: mol_type = genomic DNA
ctagcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaat

SEQ ID NO: 67 - DNA, 324 bp, Homo sapiens
  source 1..324: mol_type = genomic DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgctaa

SEQ ID NO: 68 - protein, 467 aa, synthetic construct [sequence omitted]

SEQ ID NO: 69 - protein, 107 aa, synthetic construct
DIQMTQSPSSLSASVGDRVTITCKFSQDIDRFLSWFQQKPGKAPKSLIYRVKRLVDGVPSRFSGSGSGTDFTLTISSLQPEDFATYYCVQYDEFPYGFGGGTKVEIK

SEQ ID NO: 70 - protein, 120 aa, synthetic construct
QVQLVQSGAEVKKPGSSVKVSCKASGYTFDRYWIEWVRQAPGQGLEWMGEILPGSGDINYNEKFKGRVTITADKSTSTAYMELSSLRSEDTAVYYCARMYYDYDQGFDLWGQGTLVTVSS

SEQ ID NO: 71 - protein, 7 aa, synthetic construct
GYTFDRY

SEQ ID NO: 72 - protein, 6 aa, synthetic construct
LPGSGD

SEQ ID NO: 73 - protein, 447 aa, synthetic construct
QVQLVQSGAEVKKPGSSVKVSCKASGYTFDRYWIEWVRQAPGQGLEWMGEILPGSGDINYNEKFKGRVTITADKSTSTAYMELSSLRSEDTAVYYCARMYYDYDQGFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK